(19) United States
(12) Patent Application Publication
(10) Pub. No.: US 2009/0232867 A1
(43) Pub. Date: Sep. 17, 2009

(54) Modified conductive surfaces having active substances attached thereto

(75) Inventors: Abraham J. Domb, Efrat (IL); Daniel Mandler, Jerusalem (IL); Ishaiahu Danziger, Jerusalem (IL); Miriam Oron, Jerusalem (IL); Regina Okner, Jerusalem (IL); Galit Shustak, Jerusalem (IL); Avi Swed, Holon (IL); Noam Tal, Rishon-LeZion (IL)

Correspondence Address: MARTIN D. MOYNIHAN d/b/a PRTSI, INC., P.O. BOX 16446, ARLINGTON, VA, 22215, US

(73) Assignee: Elutex Ltd., Kiryat-Shmona (IL)

(21) Appl. No.: 11/632,697

(22) PCT Filed: Jul. 19, 2005
(86) PCT No.: PCT/IL2005/000769

§ 371 Date: Jan. 18, 2007

Related U.S. Application Data
(60) Provisional application No. 60/588,749, filed on Jul. 19, 2004.

(51) Int. Cl. A61F 2/00; B05D 7/24; B05C 3/02; A61L 27/34; A61L 27/54

(52) U.S. Cl. 424/423; 427/2.24; 118/423

(57) ABSTRACT

Novel processes for coating metal surfaces and/or for attaching active substances to metal surfaces, objects having coated metal surfaces and/or active substances attached thereto and uses thereof in the preparation of implantable devices are disclosed.

### FIELD AND BACKGROUND OF THE INVENTION

The present invention relates to modified surfaces of various objects and to uses thereof and, more particularly, to such modified surfaces which can be utilized for efficiently attaching thereto organic films and/or therapeutic agents and can therefore be beneficially used in a variety of medical and other applications.

In the field of medicine, metal structures are often implanted in a living body for various purposes. Such metal structures include, for example, pacemakers, grafts, stents, wires, orthopedic implants, implantable diffusion pumps and heart valves. One problem associated with metals implanted in a living body is the biocompatibility thereof, and more particularly, the blood compatibility and the tissue compatibility of metal implants. An implant is typically considered blood biocompatible when activation of coagulation factors (e.g., proteins and platelets) is only mildly induced thereby and tissue compatible when cell proliferation and chronic inflammation are not excessively induced thereby.

However, in many applications, the metal surface, due to its hydrophilic nature, is eventually covered with a layer of adsorbed biological materials, especially proteins, from the surrounding tissues and fluids. The adsorbed layer of biological material has been implicated as being the cause of undesired biological reactions including thromboses and inflammations. Pathogenic bacterial, whether directly adhering to the metal surface or attracted by the adsorbed layer, tend to colonize the surface of such devices, turning the devices into the foci of infections. Thus, the hydrophilic nature of the metal surface is the direct cause of the failure of implants. Implant failures are medically harmful, potentially fatal, and more often than not, require unpleasant, dangerous and expensive additional surgery.

A number of strategies have been developed for overcoming these disadvantages, the main and common goal thereof being modifying the hydrophilic nature of metal surfaces. Details of the strategies and reviews thereof are found, for example, in U.S. Pat. Nos. 5,069,899, 6,617,142, 4,979,959, 3,959,078, 4,007,089, 5,024,742 and 5,024,742.

One strategy for minimizing undesirable biological reactions associated with metal implants is to coat the metal surface with biomolecules that provide a substrate for the growth of a protective cell layer. Biomolecules used include, for example, growth factors, cell attachment proteins, and cell attachment peptides. A related strategy is to attach molecules or active pharmaceutical ingredients that reduce undesired biological reactions such as antithrombogenics, antiplatelet agents, anti-inflammatory agents, antimicrobials, and the like.

A number of approaches have been provided for attaching biomolecules and other beneficial substances (henceforth collectively termed “active substances”) to metal surfaces, so as to increase the biocompatibility of the metals.

One approach involves the covalent attachment of a linking moiety to the metal surface, followed by the covalent attachment of the desired active ingredient to the linking moiety. One active ingredient that has been attached to a metal surface by a covalent bond through a linker is the anticoagulant heparin. In the Hepacoat™ stent (Cordis, a Johnson and Johnson company), heparin is covalently bonded to the stent surface and remains bonded to the stent subsequent to the implantation. The desired effect occurs by interaction in the blood stream.

Another approach involves coating a metal surface with a layer configured to form ionic bonds with an active ingredient. U.S. Pat. No. 4,442,133, for example, teaches a tridodecyl methyl ammonium chloride layer that forms ionic bonds with antibiotic agents. U.S. Pat. No. 5,069,899 teaches of a metal surface coated by a layer to which an anionic heparin is attached via an ionic bond.

Another approach involves coating a metal surface with a polymer, and trapping within the polymer a bioactive agent. Once implanted, the active pharmaceutical ingredient diffuses out of the polymer coating causing a desired effect. In the Cypher™ stent, for example, the cytostatic Sirolimus (Wyeth Pharamceuticals) is trapped within a polymer layer coating the stent. Once implanted, the active pharmaceutical ingredient diffuses out of the polymer layer, limiting tissue overgrowth of the stent. The disadvantage of such an implant is that the rate of diffusion of the active pharmaceutical ingredient from the polymer coating is neither controllable nor predictable. Further, this strategy is limited to active pharmaceutical ingredients that may be efficiently entrapped in the polymer yet able to leach out at a reasonable rate under physiological conditions.

Coating conductive surfaces such as metal surfaces using electropolymerizable monomers is highly advantageous, since it enables to control the physical and chemical properties of the coated metal surface, by controlling parameters of the electrochemical polymerization process. Electropolymerizable monomers are known in the art and include, for example, anilines, indoles, naphthalenes, pyrroles and thiophenes. When oxidized in the proximity of a surface under electropolymerization conditions, such compounds polymerize to form a polymer film of up to about 15 micron thick. Such a polymer film, although not covalently bonded to the surface, is typically bound to the surface by filling crevices, niches and gaps present in the surface. Although this film can be peeled off with relative ease, when care is taken the film remains attached to the surface. Such films are widely used in the art as a protective layer for metal surfaces, used, for example, as biosensors, (see, for example, U.S. Pat. No. 4,548,696).

Implantable medical devices loaded with active substances by means of electropolymerized films have been taught. For example, WO 99/03517, which is incorporated by reference as if fully set forth herein, teaches the ionic bonding of antisense oligonucleotides to a metal surface. In the Journal of Biomedical Materials Research vol. 44, 1999, pp. 121-129 is taught the cationic bonding of heparin to a metal surface.

WO 01/39813, which is also incorporated herein by reference as if fully set forth herein, teaches the attachment of active pharmaceutical ingredients to a surface using electropolymerizable monomers by covalent bonding of active pharmaceutical ingredients or active pharmaceutical ingredient carrying entities to electropolymerizable monomers prior to polymerization and by providing electropolymerizable monomers having functional groups which, subsequent to film production through electropolymerization, are used to covalently or ionically attach active pharmaceutical ingredients or active pharmaceutical ingredient carrying entities.

Hence, it is well recognized in the art that modifying the surface of medicinal metal structures is highly advantageous, so as to enhance the biocompatibility of such structures and to provide them with further therapeutic characteristics. The prior art teaches various strategies to overcome the limitations associated with metal implantable devices, which typically involve attachment of active substances either directly or indirectly to the metal surface. The latter includes attachment of the active substances to linker molecules or polymers via various chemical interactions (e.g., covalent or ionic bonding, encapsulation, etc.). However, the presently known strategies are limited by poor adhesion of the active substances, the linkers or the polymers to which they are attached to the metal surface.

There is thus a widely recognized need for, and it would be highly advantageous to have, metal surfaces having organic molecules (e.g., active ingredients and/or organic films) attached thereto devoid of the above limitations.

### SUMMARY OF THE INVENTION

According to one aspect of the present invention there is provided an article-of-manufacture comprising an object having a conductive surface and at least one active substance being attached to at least a portion of the conductive surface, wherein the conductive surface is a modified conductive surface having at least one functional moiety capable of interacting with the at least one active substance and/or the at least one active substance is electrochemically attached to the conductive surface.

According to further features in preferred embodiments of the invention described below, the object is a medical device, particularly an implantable device such as, for example, a pacemaker, a graft, a stent, a wire, an orthopedic implant, an implantable diffusion pump, an injection port and a heart valve. Preferably, the implantable device is a stent.

According to still further features in the described preferred embodiments the conductive surface comprises at least one metal or an alloy thereof. The metal can be, for example, iron, stainless steel, titanium, nickel, tantalum, platinum, gold, silver, copper and any combination thereof. Preferably, the conductive surface comprises stainless steel.

According to still further features in the described preferred embodiments the active substance is selected from the group consisting of a bioactive agent, a polymer, a polymer having a bioactive agent attached thereto, a plurality of microparticles and/or nanoparticles, a plurality of microparticles and/or nanoparticles having a bioactive agent attached thereto, and any combination thereof.

According to still further features in the described preferred embodiments the bioactive agent is a therapeutically active agent and/or a labeling agent.

According to still further features in the described preferred embodiments the therapeutically active agent is selected from the group consisting of an anti-thrombogenic agent, an anti-platelet agent, an anti-coagulant, a growth factor, a statin, a toxin, an antimicrobial agent, an analgesic, an anti-metabolic agent, a vasoactive agent, a vasodilator agent, a prostaglandin, a hormone, a thrombin inhibitor, an enzyme, an oligonucleotide, a nucleic acid, an antisense, a protein, an antibody, an antigen, a vitamin, an immunoglobulin, a cytokine, a cardiovascular agent, endothelial cells, an anti-inflammatory agent, an antibiotic, a chemotherapeutic agent, an antioxidant, a phospholipid, an anti-proliferative agent, a corticosteroid, a heparin, a heparinoid, albumin, a gamma globulin, paclitaxel, hyaluronic acid and any combination thereof.

According to still further features in the described preferred embodiments, the conductive surface is modified by electrochemically and/or non-electrochemically attaching thereto at least one organic substance.

According to still further features in the described preferred embodiments, the at least one organic substance forms a self-assembled monolayer onto the conductive surface.

According to still further features in the described preferred embodiments, the conductive surface is an electrochemically modified conductive surface having at least one functional moiety capable of interacting with the active substance.

According to still further features in the described preferred embodiments, the conductive surface is a non-electrochemically modified conductive surface having at least one functional moiety capable of interacting with the active substance.

According to still further features in the described preferred embodiments the interacting is effected by a covalent bond, a biodegradable bond, an ionic bond, a hydrogen bond, Van der Waals interactions, hydrophobic interactions, swelling and absorption.

According to still further features in the described preferred embodiments the at least one functional moiety is selected from the group consisting of amine, ammonium ion, carboxylate, thiocarboxylate, amide, carbamyl, hydroxyl, thiohydroxyl, alkoxide, thioalkoxide, nitrate, cyanate, pyrrole, isocyanate, halide, azide, azide, an unsaturated moiety, a hydrophobic moiety, phosphate, phosphonate, sulfate, sulfonate, sulfonamide, and any combination thereof.

According to still further features in the described preferred embodiments the conductive surface is electrochemically modified by electrochemically attaching thereto at least one organic substance, the organic substance comprising an electroattachable group and a functional moiety capable of interacting with the active substance.

According to still further features in the described preferred embodiments the electroattachable group is selected from the group consisting of a carboxylate, a sulfonate, a sulfate, a phosphonate and a phosphate.

According to still further features in the described preferred embodiments the organic substance further comprises an organic residue having 3-30 carbon atoms.

According to still further features in the described preferred embodiments the organic substance is selected from the group consisting of a fatty acid and a fatty acid derivatized by the functional group.

According to still further features in the described preferred embodiments the conductive surface is non-electrochemically modified by attaching thereto an organosilane.

Preferably, the organosilane has the general formula: XmSiR(4-m), whereas: m is an integer from 1 to 3; X is selected from the group consisting of halide, alkoxy and thioalkoxy; and R is a substituted or unsubstituted, saturated or unsaturated hydrocarbon residue. Preferably, the hydrocarbon residue has from 1 to 10 carbon atoms.

According to still further features in the described preferred embodiments, the active substance is electrochemically attached to the conductive surface.

According to still further features in the described preferred embodiments the active substance is an electropolymerized polymer.

According to still further features in the described preferred embodiments the electropolymerized polymer comprises a bioactive agent, as described herein, attached thereto.

According to still further features in the described preferred embodiments the electropolymerized polymer comprises a plurality of microparticles and/or nanoparticles attached thereto.

According to still further features in the described preferred embodiments the plurality of microparticles and/or nanoparticles comprises a bioactive agent, as described herein, being attached thereto.

According to still further features in the described preferred embodiments the electropolymerized polymer comprises a co-polymer attached thereto.

According to still further features in the described preferred embodiments the co-polymer comprises a bioactive agent, as described herein, being attached.

According to still further features in the described preferred embodiments the electropolymerized polymer is selected from the group consisting of polypyrrole, polythiophene, poly-p-phenylene, poly-p-phenylene sulfide, polyaniline, poly(2,5-thienylene), fluoroaluminum, fluorogallium, phtalocyanine, derivatives thereof and any combination thereof.

A preferred article-of-manufacture according to the present invention comprises an object having a conductive surface and a self-assembled monolayer of at least one organic substance being attached to at least a portion of the conductive surface, wherein the organic substance comprises an electroattachable group and the self-assembled monolayer is electrochemically formed onto the conductive surface.

According to another aspect of the present invention there is provided a process of preparing an object having a conductive surface and at least one active substance being attached to at least a portion of the conductive surface, as described hereinabove. The process comprises providing an object having a conductive surface; electrochemically or non-electrochemically modifying the conductive surface to thereby provide an object having a conductive surface having at least one functional moiety attached thereto, the at least one functional moiety being capable of interacting with the at least one active substance; and contacting the active substance and the conductive surface having at least one functional moiety attached thereto.

According to further features in preferred embodiments of the invention described below, the modifying is effected by electrochemically attaching to the conductive surface at least one organic substance, the organic substance comprising an electroattachable group and a functional moiety capable of interacting with the active substance, as described hereinabove.

According to further features in preferred embodiments of the invention described below, the modifying is effected by attaching to the conductive surface at least one organosilane, as detailed herein, which comprises a functional moiety capable of interacting with the active substance, as described hereinabove.

According to still further features in the described preferred embodiments the contacting is effected by reacting the object having a conductive surface having at least one functional moiety attached thereto and the active substance.

According to still further features in the described preferred embodiments the contacting is effected by swelling the active substance within the conductive surface having at least one functional moiety attached thereto.

According to still further features in the described preferred embodiments the active substance is a polymer and the contacting is effected by polymerizing a monomer corresponding to the polymer onto the conductive surface having at least one functional moiety attached thereto.

According to still further features in the described preferred embodiments the polymer is an electropolymerizable polymer and the contacting is effected by polymerizing an electropolymerizable monomer corresponding to the polymer onto the conductive surface having at least one functional moiety attached thereto.

According to still further features in the described preferred embodiments the contacting is effected by absorbing the active substance to the conductive surface having at least one functional moiety attached thereto.

According to yet another aspect of the present invention there is provided another process of preparing an object having a conductive surface and at least one active substance being attached to at least a portion of the conductive surface, as described hereinabove. The process comprises providing an object having a conductive surface; and electrochemically attaching to the conductive surface at least one active substance having an electroattachable group.

According to further features in preferred embodiments of the invention described below, the process further comprises, prior to the electrochemically attaching, modifying the conductive surface, to thereby provide an object having a conductive surface having at least one functional moiety capable of interacting with the at least one active substance, as is described hereinabove.

According to still another aspect of the present invention there is provided a method of treating a subject having a medical condition in which implanting a medical device is beneficial. The method comprises providing a medical device having a conductive surface and an active substance being attached at least to a portion of the conductive surface, wherein the conductive surface is a modified conductive surface having at least one functional moiety capable of interacting with the at least one active substance and/or the at least one active substance is electrochemically attached to the conductive surface, as is detailed hereinabove, and implanting the medical device within the subject, thereby treating the medical condition.

According to further features in preferred embodiments of the invention described below, the medical condition is selected from the group consisting of a cardiovascular disease, atherosclerosis, thrombosis, stenosis, restenosis, a cardiologic disease, a peripheral vascular disease, an orthopedic condition, a proliferative disease, an infectious disease, a transplantation-related disease, a degenerative disease, a cerebrovascular disease, a gastrointestinal disease, a hepatic disease, a neurological disease, an autoimmune disease, and an implant-related disease.

According to an additional aspect of the present invention there is provided a system for coating at least one medical device having a conductive surface, the system comprising in operative arrangement, at least one holding device for holding the medical device, a conveyer, and a first and a second bath arranged along the conveyer, wherein the conveyer is designed and constructed to convey the at least one holding device such that the at least one holding device is placed within each of the first and second baths for a predetermined time period and in a predetermined order, and further wherein the first bath is a modification bath and the second bath is an active substance solution bath.

According to further features in preferred embodiments of the invention described below, the modification bath comprises an organic substance having a functional moiety capable of interacting with the active substance.

According to still further features in the described preferred embodiments the active substance is an electropolymerized polymer and the second bath is an electropolymerization bath.

According to still further features in the described preferred embodiments the at least one medical device comprises at least one stent assembly.

According to still further features in the described preferred embodiments the system further comprises at least one additional bath arranged along the conveyer, wherein the conveyer is designed and constructed to place the at least one holding device within the at least one additional treating bath for a predetermined time period.

According to still further features in the described preferred embodiments the at least one additional treating bath is selected from the group consisting of a pretreatment bath, a washing bath, a rinsing bath, an electropolymerization bath, a chemical polymerization bath and a second active substance solution bath.

According to still further features in the described preferred embodiments the system further comprises a cartridge having a cartridge body adapted for enabling the at least one holding device to be mounted onto the cartridge body.

According to still further features in the described preferred embodiments the at least one holding device comprises a perforated encapsulation, adapted to receive the at least one medical device, and at least two cups adapted for enabling electrode structures to engage with the perforated encapsulation hence to generate an electric field within the perforated encapsulation.

According to still further features in the described preferred embodiments the perforated encapsulation is designed and constructed to allow fluids and chemicals to flow therethrough.

According to still further features in the described preferred embodiments the electropolymerization bath comprises at least one electrode structure, mounted on a base of the electropolymerization bath and connected to an external power source.

According to still further features in the described preferred embodiments the conveyer is operable to mount the at least one holding device on the at least one electrode structure, thereby to engage the at least one electrode structure with a first side of the perforated encapsulation.

According to still further features in the described preferred embodiments the system further comprises an arm carrying at least one electrode structure and operable to engage the at least one electrode structure with a second side of the perforated encapsulation.

The present invention successfully addresses the shortcomings of the presently known configurations by providing novel processes for coating metal surfaces, which result in stable, uniform and adherent coatings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents the first and second cyclic voltammograms of 316L stainless steel in a solution of 0.1 mM decanoic acid and 0.1 M TBATFB in acetonitrile, with an applied scan rate of 100 mV·sec⁻¹;

FIG. 2 presents the cyclic voltammetry of 1 mM Ru(NH₃)₆³⁺ in 0.1 M NaCl recorded with a bare 316L stainless steel electrode (a), a 316L stainless steel electrode electrochemically modified in 0.1 mM palmitic acid and 0.1 M TBATFB/ACN solution after 1 cycle (b), after 5 cycles (c) and after 10 cycles (d), while applying a scan rate of 100 mV·sec⁻¹;

FIG. 3 presents the cyclic voltammetry of 1 mM Ru(NH₃)₆³⁺ in 0.1 M NaCl (scan rate 100 mV·s⁻¹) recorded with bare 316L stainless steel electrode (a), a 316L stainless steel electrode electrochemically modified in 0.1 M TBATFB/ACN and 0.1 mM of decanoic acid (b), myristic acid (c) and palmitic acid (d), for 10 cycles;

FIG. 4 presents the cyclic voltammetry of 1 mM Ru(NH₃)₆³⁺ in 0.1 M NaCl (scan rate 100 mV·s⁻¹) recorded with a freshly polished 316L stainless steel electrode (a), a 316L stainless steel electrode electrochemically cycled 10 times in 0.1 M TBATFB/ACN solution (b), and a 316L stainless steel electrode polished and left under ambient conditions for one day (c);

FIGS. 5a-b present the cyclic voltammetry of 1 mM Ru(NH₃)₆³⁺ in 0.1 M NaCl recorded with a 316L stainless steel electrode electrochemically cycled 10 times in 0.1 M TBATFB/ACN solution containing 0.1 mM decanoic acid at a scan rate of 10 (a), 100 (b), 200 (c), 500 (d), 1000 (e), and 2000 (f) 2000 mV·sec⁻¹, whereby the inset shows the dependence of the peak current as a function of square root of the scan rate (FIG. 5a) and the cathodic peak potential as a function of the logarithm of the scan rate (FIG. 5b);

FIG. 6 presents the double-layer capacity as a function of the potential measured in 0.1M NaNO₃ solution for a freshly polished bare 316L stainless steel electrode (left-pointing triangles) and in a 316L stainless steel electrode electrochemically cycled 10 times in 0.1 M TBATFB/ACN solution (squares), a 316L stainless steel electrode electrochemically cycled 10 times in 0.1 M TBATFB/ACN solution containing 0.1 mM decanoic (circles), 0.1 mM myristic (up-pointing triangles), 0.1 mM palmitic (down-pointing triangles), and 0.1 mM stearic acid (diamonds);

FIG. 7 presents the dependence of the reciprocal double-layer capacity illustrated in FIG. 6, as a function of the length of the acid (decanoic acid, myristic acid, palmitic acid and stearic acid);

FIG. 8 presents the reflection absorption FTIR spectra of the C—H stretching region of a 316L stainless steel electrode electrochemically cycled 10 times in 0.1 M TBATFB/ACN solution containing 0.1 mM decanoic (solid line), 0.1 mM myristic (dash-dotted line), and 0.1 mM palmitic (dashed line);

FIG. 9 presents the reflection absorption FTIR spectra of the carbonyl stretching-region of a 316L stainless steel surface treated with palmitic acid;

FIGS. 10a-b present the high resolution XPS spectra of Fe 2p_{3/2} and Fe2p_{1/2} (FIG. 10a) and O1s (FIG. 10b) of a freshly polished bare 316L stainless steel electrode (denoted as 1), a 316L stainless steel electrode electrochemically cycled 10 times in 0.1 M TBATFB/ACN solution (denoted as 2), and a 316L stainless steel electrode electrochemically cycled 10 times in 0.1 M TBATFB/ACN solution containing 0.1 mM palmitic acid (denoted as 3);

FIGS. 11a-b are pictures presenting a bare 316L stainless steel plate after electropolymerization of pyrrole thereon (FIG. 11a, right hand) and a permacel 99 tape obtained after a cross-cut tape adhesion test therewith (FIG. 11a, left hand), and a 316L stainless steel electrode subjected to SAM formation in the presence of decanoic acid and thereafter to electropolymerization of pyrrole (FIG. 11b, right hand) and a permacel 99 tape obtained after a cross-cut tape adhesion test therewith (FIG. 11b, left hand);

FIGS. 12a-c present SEM micrographs of a bare 316L stainless steel plate (FIG. 12a), a 316L stainless steel plate electrocoated by polypyrrole by scanning the potential 10 times between −0.8 and 1.25 V vs. Ag|AgBr with 100 mV·s⁻¹ scan rate in 0.1 M pyrrole in acetonitrile (FIG. 12b), and a 316L stainless steel plate subjected to SAM formation in the presence of decanoic acid, washed and thereafter electrocoated with polypyrrole (FIG. 12c);

FIGS. 13a-b present a schematic illustration of a 12-aminododecanoic acid SAM formed on a 316L stainless steel plate (FIG. 13a) and the XPS spectrum of a 316L stainless steel plate treated with 12-amino dodecanoic acid (FIG. 13b);

FIG. 14 presents the ¹H NMR spectrum of a pyrrole-functionalized copolymer used in the preparation of surface-functionalized nanoparticles, according to a preferred embodiment of the present invention;

FIGS. 15a-c present SEM micrographs of a 316L stainless plate pre-treated with decanoic acid and electrocoated by pyrrole-substituted nanoparticles;

FIGS. 16a-c present SEM micrographs of a stainless steel 316 LM stent (STI, Israel, 12×1 mm) electrocoated with decanoic acid SAMs and pyrrole-substituted nanoparticles;

FIG. 17a-d present SEM micrographs of a 316L stainless plate pre-treated with 12-aminododecanoic acid and having PLA particles electrostatically attached thereto, prepared by incubation of the pre-treated plate with a buffer solution containing the dispersed particles at room temperature;

FIG. 18 presents comparative plots demonstrating the release of Taxol from stainless steel devices (20×10 mm²) coated with poly(butyl ester)pyrrole on decanoic acid (blue) and poly(ethyl ester)pyrrole on decanoic acid (violet);

FIG. 19 presents a schematic illustration of a formation of biotin-avidin complexes onto metal surfaces, according to an embodiment of the present invention;

FIG. 20 present a schematic illustration of a formation of a SAM of an exemplary organosilane formed on a stainless steel surface according to an embodiment of the present invention;

FIG. 21 is a schematic representation of an exemplary holding device, according to the present embodiments;

FIG. 22 is a schematic representation of an exemplary cartridge according to the present embodiments; and

FIG. 23 is a schematic representation of an exemplary system, according to the present embodiments.

### DESCRIPTION OF THE PREFERRED EMBODIMENTS

The present invention is of novel coatings of conductive surfaces, which are characterized by enhanced adherence of the coating to the surface and thus efficiently enhance the biocompatibility of the surface and can therefore be beneficially used as coatings of medical devices, particularly implantable devices.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is discussed hereinabove, medical devices having a metal surface are often characterized by poor biocompatibility due to their hydrophilic nature. Strategies developed to enhance the biocompatibility of such devices include coating the metal surface by a hydrophobic layer, which may optionally further include a bioactive agent (e.g., a drug). While the prior art teaches various methods of attaching hydrophobic moieties to metal surfaces, these methods are typically limited by poor adhesion of the coating and/or uncontrolled release of the bioactive agents therefrom.

Considerable efforts are therefore aimed at the development of “molecular adhesion promoters” that improve the linkage between a metal substrate and an organic coating [1-4]. The adhesion between hydrophobic organic films and hydrophilic metal surfaces is expected to be insufficient for applications, such as coating of implantable medical devices for the purpose of increasing their biocompatibility. Among metals, stainless steel is of special importance due to its use in orthopedic implants and other implantable medical devices, owing to its corrosion resistance and superior mechanical properties [5]. The biocompatibility of stainless steel implants can be significantly improved by modifying its surface with organic molecules or polymers [6-9]. With the increased interest in drug eluting medical devices in general and stents in particular [10], where metallic surfaces are coated with a drug-loaded polymer, adherent and uniform thin coating (1-2 μm) are desired. Coating of devices by polymer solution, by either dipping or spraying, typically results in a thick coating (about 15 μm) with limited uniformity and adherence.

While conceiving the present invention, it was envisioned that (i) electrochemical functionalization of metal surfaces, which results in a surface having free functional groups attached thereto, can dramatically improve the subsequent attachment of a hydrophobic layer thereto in a controllable way; and (ii) that such a functionalization can be advantageously performed by forming self-assembled monolayers of an organic substance onto the surface.

Deposition of self-assembled monolayers (SAMs) is considered to be an advantageous method for creating chemically well-defined surfaces on solid supports and in particularly on metal surfaces [7-9]. In spite of the increasing interest and efforts devoted to self-assembly technology in recent years, only a few reports dealing with SAMs on stainless steel have been published [2-5, 10-13]. In one example, the formation and characterization of n-alkanethiol SAMs on electrochemically reduced stainless steel surfaces was performed by applying a negative potential in order to remove the native oxide layer of stainless steel, followed by the addition of thiols to the electrolyte solution [4,5]. Other carboxylic acid monolayers have been assembled on iron [1,4] and steel [1,5] using Langmuir-Blodgett (LB) as a mean of corrosion inhibition and increasing lubrication of the surface. These studies all involve pre-activation of the stainless steel surface by electrochemical means and thereafter deposit the monolayer thereon.

In general, there are two principal possible interactions between an n-alkanoic acid monolayer and a metal surface: ionic and covalent [1,8]. In several studies conducted in this respect, it was mostly concluded that the formation of acid monolayers on metal surfaces involves an acid-base interaction that results in a metal carboxylate salt [16-17]. Other studies involving deposition of long-chain n-alkanoic acids on reactive metals having a native oxide overlayer, such as aluminum [20-22], silver [22-25] and copper [22-23, 25], suggest that the acids are probably chemisorbed via proton transfer to a surface oxygen atom to form ionic bonds.

Mono-functional silylating agents have also been used to form monolayer surface coatings, while di- and tri-functional silylating agents have been used to form polymerized coatings on silica surfaces. Many silylating agents, however, produce coatings with undesirable properties including instability to hydrolysis and the inadequate ability to mask the silica surface which may contain residual acidic silanols.

It was thus envisioned by the present inventor(s) that deposition of SAMs on metal surfaces, using organic substances that may further interact with various substances, would result in a well-ordered and well-defined thin layer that can act as an efficient adherent layer for attaching variable agents and/or films to the metal surface.

While reducing the present invention to practice, it was indeed found that forming SAMs on metal surfaces, particularly stainless steel surfaces, enables the attachment of variable substances and thus results in highly dense and highly adherent coatings of the surface. It was further found that using such an approach, versatile coatings having versatile characteristics can be controllably prepared. A novel approach for forming SAMs on stainless steel surfaces was also developed.

As is exemplified in the Examples section that follows, self-assembled monolayers of various fatty acids and of organosilanes deposited on a native oxide surface of 316L stainless steel were prepared and characterized. The fatty acid SAMs were prepared using a novel approach for modifying stainless steel surfaces, in which the self-assembly of n-alkanoic acids is facilitated by applying a potential to the stainless steel in an organic electrolyte solution. This novel approach results in highly reproducible monolayers that are deposited within a shorter time than the presently known assembly processes.

The organosilane SAMs were prepared by simply dipping the stainless steel surfaces in diluted organosilane solutions.

As is further exemplified in the Examples section that follows, thus modified stainless steel surfaces were further interacted with various active substances, for example, electropolymerizable monomers and/or various functionalized nanoparticles and polymers, resulting in uniform, dense and highly adherent coatings of the surfaces. The bioactive substances interacted either directly with the surface, or via any of the resulting coatings of the surfaces, as is detailed hereinunder.

Thus, according to one aspect of the present invention, there is provided an article-of-manufacture, which comprises an object having a conductive surface and an active substance being attached to at least a portion of the surface. The conductive surface, according to the present invention, can be an electrochemically modified conductive surface having one or more functional moieties that are capable of interacting with the active substance, whereby the active substance can be electrochemically attached to the metal surface, either directly or indirectly, by pre-modifying the surface.

While, as is discussed hereinabove, modifying a hydrophilic metal surface of an object is highly beneficial in medical devices, particularly implantable medical devices, the object is preferably a medical device. The medical device can be any medical device that comprises a metal surface and includes, for example, extra corporeal devices such as aphaeresis equipment, blood handling equipment, blood oxygenators, blood pumps, blood sensors, fluid transport tubing and the like. However, modifying a hydrophilic metal surface is particularly useful in implantable medical devices such that the medical device can be an intra corporeal device such as, but not limited to, aortic grafts, arterial tubing, artificial joints, blood oxygenator membranes, blood oxygenator tubing, bodily implants, catheters, dialysis membranes, drug delivery systems, endoprostheses, endotracheal tubes, guide wires, heart valves, intra-aortic balloons, medical implants, pacemakers, pacemaker leads, stents, ultrafiltration membranes, vascular grafts, vascular tubing, venous tubing, wires, orthopedic implants, implantable diffusion pumps and injection ports.

Particularly preferred medical devices according to the present invention are stents, and more particularly expandable stents. Such stents can be of various types, shapes, applications and metal compositions and may include any known stents. Representative examples include the Z, Palmaz, Medivent, Strecker, Tantalum and Nitinol stents.

The phrase “implantable device” is used herein to describe any medical device that is placed within a bodily cavity for a prolonged time period.

Suitable conductive surfaces for use in the context of the present invention include, without limitation, surfaces made of one or more metals or metal alloys. The metal can be, for example, iron, steel, stainless steel, titanium, nickel, tantalum, platinum, gold, silver, copper, any alloys thereof and any combination thereof. Other suitable conductive surfaces include, for example, shape memory alloys, super elastic alloys, aluminum oxide, MP35N, elgiloy, haynes 25, stellite, pyrolytic carbon and silver carbon.

Since particularly useful objects are implantable medical devices, and further since such devices are typically made of stainless steel, the conductive surface preferably comprises stainless steel.

As is discussed in detail hereinabove, medical devices having metal surfaces in general and stainless steel surfaces in particular suffer many disadvantages, mostly due to the poor blood and/or tissue biocompatibility of such surfaces. As is further discussed hereinabove, poor blood biocompatibility typically results in activation of coagulation proteins and platelets whereby poor tissue biocompatibility typically results in excessive cell proliferation and inflammation. Modifying the surface so as to enhance its biocompatibility can be performed by chemical and/or physical means that are aimed at improving the surface characteristics in terms of charge, wettability and topography. These can be achieved by attaching to the surface a thin layer (film) of substances such as polymers (e.g., poly(ethylene glycol), Teflon and polyurethane). Alternatively, modifying the surface can be performed by attaching a bioactive agent to the surface, which can reduce the adverse effects associated with the poor biocompatibility or can induce additional beneficial effects.

Thus the conductive surface, according to the present invention, has one or more active substances attached to at least a portion thereof.

The phrase “active substance” is used herein to describe any substance that may affect the surface chemical and/or physical characteristics and includes, for example, substances that affect the charge, wettability, and topography of the surface, substances that reduce the adverse side effects induced by the surface and/or pharmaceutically active ingredients that may provide the object with additional therapeutic effect.

Hence, preferred active substances, according to the present invention, include, without limitation, bioactive agents, polymers, polymers having a bioactive agent attached thereto, microparticles, nanoparticles, microparticles and/or nanoparticles having a bioactive agent attached thereto, and any combination thereof.

The phrase “bioactive agent” is used herein to describe an agent capable of exerting a beneficial activity in a subject. Such a beneficial activity include, as is discussed hereinabove, reducing adverse side effects induced by the surface and/or any other therapeutic activity, depending on the condition being treated by the medical device.

The bioactive agent can therefore be a therapeutically active agent, which is also referred to herein interchangeably as a pharmaceutically active agent or an active pharmaceutical agent.

The bioactive agent can further be a labeling agent, which may serve for detecting and/or locating the substance to which it is attach in the body and may be used, for example, for diagnosis and follow-up purposes.

The phrase “labeling agent” is therefore used herein to describe a detectable moiety or a probe and includes, for example, chromophores, fluorescent compounds, phosphorescent compounds, heavy metal clusters, and radioactive labeling compounds, as well as any other known detectable moieties.

In some cases, the therapeutically active agent may be labeled and thus further serve as a labeling agent. Similarly, some labeling agents, such as radioisotopes, as also serve as therapeutically active agents.

Polymers and particles such as nanoparticles and microparticles can be applied per se onto a surface, so as to affect its physical, mechanical and chemical characteristics, as described hereinabove, whereby bioactive agents are applied so as to affect the surface's biological characteristics. Polymers and particles having a bioactive agent attached thereto are typically applied onto a surface so as to affect its physical and chemical characteristic and on the same time to act as carriers of one or more bioactive agents.

Polymers and particles that serve as carriers of a bioactive agent can be either stable or biodegradable when applied. The term “biodegradable” is used to describe such materials that may be decomposed upon reaction with e.g., enzymes (hydrolases, amidases, and the like), whereby the term “stable” is used to describe such materials that remain intact when applied, at least for a prolonged time period. The release of the bioactive agent from a stable carrier is typically performed by diffusion of the agent.

The objects, according to the present invention, can include a combination of a bioactive agent biodegradably attached to the surface, which is further coated by a polymer. The bioactive agent can be released from the object, if needed, by diffusion through the polymer. Optionally, the objects, according to the present invention, can include a combination of a bioactive agent biodegradably attached to any of the surface coatings or SAMs, which may further be coated by a polymer.

The phrase “having a bioactive agent being attached thereto” with respect to polymers, particles and any other moiety mentioned herein, is used herein to describe any form in which the bioactive agent is attached to the moiety and therefore includes covalent attachment, by either biodegradable bonds or stable bonds, attachment by electrostatic interactions (e.g., ionic bonds), encapsulation, swelling, absorption and any other acceptable attachment form.

The bioactive agent being attached to the surface can be selected according to the condition being treated by the medical device. Representative examples of bioactive agents with are useful in the context of the present invention include, without limitation, anti-thrombogenic agents, anti-platelet agents, anti-coagulants, statins, toxins, growth factors, antimicrobial agents, analgesics, anti-metabolic agents, vasoactive agents, vasodilator agents, prostaglandins, hormones, thrombin inhibitors, enzymes, oligonucleotides, nucleic acids, antisenses, proteins (e.g., plasma proteins, albumin, cell attachment proteins, biotin and the like), antibodies, antigens, vitamins, immunoglobulins, cytokines, cardiovascular agents, endothelial cells, anti-inflammatory agents (including steroidal and non-steroidal), antibiotics (including antiviral agents, antimycotics agents and the like), chemotherapeutic agents, antioxidants, phospholipids, anti-proliferative agents, corticosteroids, heparins, heparinoids, albumin, gamma globulins, paclitaxel, hyaluronic acid and any combination thereof.

Bioactive agents such as anti-thrombogenic agents, anti-platelet agents, anti-coagulants, statins, vasoactive agents, vasodilator agents, prostaglandins, thrombin inhibitors, plasma proteins, cardiovascular agents, endothelial cells, anti-inflammatory agents, antibiotics, antioxidants, phospholipids, heparins and heparinoids are particularly useful when the medical device is a stent. Bioactive agents such as analgesics, anti-metabolic agents, antibiotics, growth factors and the like, are particularly useful when the medical device is an orthopedic implant.

Non-limiting examples of commonly used statins include Atorvastatin, Fluvastatin, Lovastatin, Pravastatin and Simvastatin.

Non-limiting examples of non-steroidal anti-inflammatory drugs include oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone.

Non-limiting examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Non-limiting examples of analgesics (pain relievers) include aspirin and other salicylates (such as choline or magnesium salicylate), ibuprofen, ketoprofen, naproxen sodium, and acetaminophen.

Growth factors are hormones which have numerous functions, including regulation of adhesion molecule production, altering cellular proliferation, increasing vascularization, enhancing collagen synthesis, regulating bone metabolism and altering migration of cells into given area. Non-limiting examples of growth factors include insulin-like growth factor-1 (IGF-1), transforming growth factor-β (TGF-β), a bone morphogenic protein (BMP) and the like.

Non-limiting examples of toxins include the cholera toxin, which also serves as an adjuvant.

Non-limiting examples of anti-proliferative agents include an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, or a gonadotropin-releasing hormone analog. Specific examples of chemotherapeutic agents include, for example, a nitrogen mustard, an epipodophyllotoxin, an antibiotic, a platinum coordination complex, bleomycin, doxorubicin, paclitaxel, etoposide, 4-OH cyclophosphamide, and cisplatinum.

In one embodiment of the present invention, the conductive surface is an electrochemically modified conductive surface having one or more functional moieties that are capable of interacting with the active substance.

According to this embodiment, the conductive surface, which is typically chemically inert, is functionalized so as to enable its interaction with various active substances.

The phrase “functional moiety” is used herein to describe a residue of a substance, preferably an organic substance, which can interact with another substance via, for example, formation of chemical bonds, chemical interactions or physical interactions therebetween. The interactions between the substances may involve either major portions of the substance (as in the case of hydrophobic interactions, absorption, swelling and the like), or a specific functional group within the substance, preferably at the distal end thereof with respect to the surface (as in the case of, for example, covalent and electrostatic bonds). The interactions can be via formation of covalent bonds, either stable or biodegradable, formation of ionic bonds, hydrogen bonds interactions, Van der Waals interactions, and hydrophobic interactions, and/or by swelling or absorption via any chemical or physical interaction.

Representative examples of functional moieties include amine, ammonium ion, carboxylate, thiocarboxylate, amide, carbamyl, hydroxyl, thiohydroxyl, alkoxide, thioalkoxide, nitrate, cyanate, azide, isocyanate, halide, azide, an unsaturated moiety, a hydrophobic moiety, phosphate, phosphonate, sulfate, sulfonate, sulfonamide, and any combination thereof.

As used herein, the term “amine” refers to a substance having or terminating with an —NR′R″, wherein each of R′ and R″ is independently hydrogen, alkyl, cycloalkyl or aryl, as is defined herein or R′ and R″ may together form a five, six or more membered carbocyclic or heterocyclic ring.

The term “ammonium ion” refers to a substance having or terminating by a positively charged —N⁺R′R″R′″ group, where R′ and R″ are as described hereinabove and R′″ is as described for R′ and R″.

The term “carboxylate” refers to a substance having or terminating with a —C(═O)Y group, where Y can be hydrogen, alkyl, cycloalkyl, aryl, hydroxyl, thiohydroxyl, halide, azide, alkoxide, thioalkoxide. This term therefore encompasses aldehydes, ketones, esters, acyl halides, amides, carboxylic acids and thio derivatives thereof.

The term “thiocarboxylate” refers to a substance having or terminating with a —C(═S)Y group, where Y can be hydrogen, alkyl, cycloalkyl, aryl, hydroxyl, thiohydroxyl, halide, azide, amine, alkoxide, thioalkoxide. This term therefore encompasses thioaldehydes, thioketones, thioesters, thioacyl halides, thioamides, thiocarboxylic acids.

The term “amide” refers to a substance having or terminating with a —C(═O)NR′R″ group, where R′ and R″ are as defined herein.

The term “carbamyl” refers to a —OC(═O)—NR′R″ group or a R″OC(═O)—NR′—, where R′ and R″ are as defined herein.

The term “alkyl” refers to a saturated aliphatic hydrocarbon including straight chain and branched chain groups. Preferably, the alkyl group has 1 to 30 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 30 carbon atoms.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl.

The term “hydroxyl” refers to a substance having or terminating with an —OH group or to an —OH group per se.

The term “azide” refers to a substance having or terminating with a —N═NR′ group, where R′ is as defined herein.

The term “alkoxide” refers to a substance having or terminating with an —O-alkyl, O-aryl or an —O-cycloalkyl group, as defined herein, or to an —O-alkyl, O-aryl and O-cycloalkyl group per se The term “thiohydroxyl” refers to a substance having or terminating with a —SH group or to a —SH group per se.

The term “thioalkoxide” refers to a substance having or terminating with a —S-alkyl group, —S-cycloalkyl group or S-aryl group, as defined herein, or to a —S-alkyl, S-aryl and S-cycloalkyl group per se.

The term “halide” refers to a substance having or terminating with a fluorine, chlorine, bromine or iodine atom or to a fluorine, chlorine, bromine or iodine atom per se.

The term “sulfate” group refers to a substance having or terminating with a —OS(═O)₂OR′ group, where R′ is as defined herein or to a OS(═O)₂OR′ group per se.

The term “sulfonate” refers to a substance having or terminating with a —S(═O)₂—OR′ group, where R′ is as defined herein, or to a —S(═O)₂OR′ group per se.

The term “sulfonamide” group refers to a substance having or terminating with a —S(═O)₂—NR′R″ or a R′S(═O)₂—NR′— group, with R′ and R″ as defined herein.

The term “nitrate” refers to a substance having or terminating with a —NO₂ group.

A “cyanate” refers to a substance having or terminating with a —C≡N group.

An “isocyanate” refers to a substance having or terminating with a —N≡CR′ group, with R′ as defined herein.

The term “phosphonate” refers to a substance having or terminating with an —O—P(═O)(OR′)₂ group, with R′ as defined hereinabove, or to an —O—P(═O)(OR′)— group per se.

The term “phosphate” refers to a substance having or terminating with a P(═O)(OR′)₂ group, with R′ as defined hereinabove, or to a —P(═O)(OR′)₂ group per-se.

The term “unsaturated moiety” refers to a substance having or terminating with an alkenyl group, a alkynyl group or an aryl group, as defined herein, and encompasses vinyls, allyls and the like.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

The term “hydrophobic moiety” refers to a substance characterized by substantial hydrophobicity and typically includes substances comprised of or having a plurality of alkyl, cycloalkyl, alkenyl, alkynyl, and/or aryl groups, optionally attached one to another.

In a preferred embodiment of the present invention, the conductive surface is electrochemically modified by electrochemically attaching thereto one or more organic substances that further comprise, in addition to the functional groups described above, one or more electroattachable groups.

The phrase “electroattachable group” is used herein to describe a group that upon application of an electric potential can be oxidized or reduced and thus form a ionic, covalent or organometallic bond with a conductive material, herein a conductive surface.

Preferred electroattachable groups according to the present invention include, for example, a carboxylic acid group, a sulfonate group, a sulfate group, a phosphonate group and a phosphate group, as these terms are defined hereinabove. Such groups are typically oxidized during an electrochemical process, to produce anions thereof that can bind a metal surface, and particularly a native oxide layer of a surface, via formation of ionic bonds.

The organic substance preferably further comprises an organic residue having 3-30 carbon atoms. Organic substances having an electroattachable end group and an organic residue may form SAMs when deposited on a conductive surface and are therefore highly beneficial, as is discussed hereinabove.

In a preferred embodiment of the present invention, the organic substance is a fatty acid. Fatty acids have been shown to form SAMs on metal surfaces. However, the formation of fatty acid SAMs on the beneficial stainless steel surfaces has never been studied before.

Representative examples of fatty acids that are usable in the context of the present invention include, without limitation, saturated fatty acids such as, for example, decanoic (capric) acid, undecanoic acid, dodecanoic (lauric) acid, tridecanoic acid, tetradecanoic (myristic) acid, pentadecanoic, hexadecanoic (palmitic) acid, heptadecanoic (margaric) acid, octadecanoic (stearic) acid, nonadecanoic, eicosanoic (arachidic) acid, docosanoic (behenic) acid, tetracosanoic (lignoceric) acid, and the like, as well as unsaturated fatty acids such as, for example, arachidonic acid, linoleic acid, linolenic acid and the like.

The fatty acid may be used as is or as a derivatized fatty acid having a functional moiety, as described above, attached thereto.

As is exemplified and detailed in the Examples section that follows, the electrochemical attachment of various fatty acids to stainless steel surfaces resulted in the formation of SAMs, whereby the order degree of the SAMs, as well as other characteristics thereof were found to be dependent on the chain length, thus enabling to control these characteristics by pre-selecting the fatty acid according to the desired characteristics.

In another preferred embodiment, the electrochemical modification is performed by contacting a conductive surface with an electrolyte solution containing the fatty acid and sweeping the potential of the surface from e.g., −0.5 V to 1.5 V, preferably from about −0.8 V to about 1.2 V, depending on the cathode used. This self-assembly technique involves modification of the surface under open-circuit potential for typically three hours to several days, and is facilitated and easily controlled, as compared with the presently known methods for depositing acids of metal surfaces, by applying a potential in the course of the modification process.

In another preferred embodiment of this aspect of the present invention, the conductive surface is a non-electrochemically modified conductive surface having one or more functional moieties that are capable of interacting with the active substance.

The phrase “non-electrochemically modified surface”, as used herein, refers to any form of modification of the surface that does not involve an electrochemical process.

Non-electrochemically modified surfaces, according to the present embodiments, preferably include one or more organic substances that are non-electrochemically attached to the surface, and have functional moieties that are capable of interacting with the organic substance.

Attachment of such organic substances therefore involve a formation of strong interactions between the organic substance and the surface, whereby the interactions can be, for example, electrostatic, coordinative or covalent bonds, or, otherwise, can be as a result of chemisorption, high affinity and the like. Suitable organic substances that can be non-electrochemically attached to metallic surfaces include, for example, organosilanes, organoboranes and the like.

In a preferred embodiment of the present invention, the organic substance is an organosilane. Organosilanes have been shown to form SAMs on metal surfaces. However, the formation of organosilane SAMs on the beneficial stainless steel surfaces has never been studied before.

The term “organosilane” as used herein describes an organic compound that has at least one silicon atom and at least one carbon atom. Preferably, the organosilane is of a general formula:

XmSiR(4-m)

wherein: m is an integer from 1 to 3; X is selected from the group consisting of halide, alkoxy and thioalkoxy; and R is a substituted or unsubstituted, saturated or unsaturated hydrocarbon residue.

Preferred organosilanes according to the present embodiments therefore include one or more silicon atoms, substituted by one or more reactive groups that are capable of interacting, as detailed hereinabove, with the surface. Such reactive groups typically include hydroxy groups. Since hydroxy silanes are highly reactive and are typically present in a form of silica, preferred organosilanes are silicon-containing compounds that have functional groups that can be easily converted, in situ, to hydroxy groups. These include, for example, halides such as chlorides and alkoxy or aryloxy groups. These groups are readily converted to the reactive hydroxy group in the presence of minute amounts of water.

Preferred organosilanes according to the present embodiments further include one or more hydrocarbon residues, as defined herein.

The hydrocarbon can be substituted or unsubstituted, saturated or unsaturated and can optionally further be interrupted by one or more heteroatoms such as O, N and/or S. When un substituted, the hydrocarbon can serve as the functional moiety, as described hereinabove, for hydrophobically interacting with the active substance, and/or absorbing or swelling the active substance. Alternatively, substituted hydrocarbons can be used, which include a functional group that can form covalent or electrostatic bonds with the active substance.

As is exemplified and described in detail in the Examples section that follows, the attachment of various organosilanes to stainless steel surfaces resulted in the formation of SAMs, whereby the order degree of the SAMs, as well as other characteristics thereof was found to be dependent on the chain length, thus enabling to control these characteristics by pre-selecting the organosilane according to the desired characteristics.

Representative examples of organosilanes that are suitable for use in this context of the present invention are presented in the Examples section that follows.

In another embodiment of this aspect of the present invention, the active substance is directly attached, electrochemically, to the conductive surface. This can be performed using active substances, as described hereinabove, which have an electroattachable group, as is described hereinabove. Such a direct attachment of a bioactive substance to a medical device enables the release of therapeutically active agents in the immediate vicinity of the medical device. Furthermore, if the bioactive agent is a labeling agent, its direct attachment to the implantable device enables the monitoring of the implantable device in the body, by e.g. following the position of the implantable device in the body.

The active substance, according to this embodiment, can therefore be a bioactive agent having, for example, a carboxylic acid, phosphate, sulfonate and the like as end groups, or polymers and particles having such groups on their surface. The preparation of the latter is described and exemplified in the Examples section that follows.

In another embodiment of this aspect of the present invention, the conductive surface is electrochemically modified so as to have functional moieties capable of interacting with the active substance, as described hereinabove and the active substance is also attached to the modified surface by electrochemical means. The modification of the surface and the electroattachment of the active substance can be performed either simultaneously or subsequently.

In a representative example of this embodiment, the active substance is an electropolymerizable polymer.

The phrase “electropolymerizable polymer” is used herein to describe a polymer that can be formed by applying a potential to a solution of its corresponding monomer or monomers. The monomer or monomers are termed herein “electropolymerizable monomers”, whereby the electropolymerization process is also referred to herein, interchangeably, “electrochemical polymerization and in some cases “electrocoating”.

The electropolymerized polymer can be applied onto the surface per se, so as to improve the surface characteristics, or as a carrier of bioactive agents which are directly or indirectly attached thereto. Thus the electropolymerized polymer can comprise microparticles and nanoparticles that optionally and preferably contain bioactive agents, or a co-polymer that optionally and preferably contain bioactive agents.

Alternatively, the electropolymerized polymer can form a polymeric film in which a bioactive agent can be absorbed, swelled or otherwise embedded.

A detailed description of various methodologies for attaching bioactive agent and other substances to electropolymerized polymers can be found in the Examples section that follows, and in a U.S. patent application, entitled “Electropolymerizable monomers and polymeric coatings on implantable devices obtained therefrom”, by the primary present inventor, which has an Attorney Docket No. 28166 and is being co-filed with the instant application, which is incorporated by reference as if fully set forth herein.

The electropolymerizable polymer can interact with the functional moieties of the modified surface by various interactions, however, in a preferred embodiment, the polymer forms hydrophobic interactions with hydrophobic moieties present onto the surface. Such hydrophobic interactions substantially enhance the adherence of the polymer to the surface.

In cases where the modification of the surface involves the formation of moleculary dimensioned, hydrophobic SAMs, the polymer film deposited thereon, can acquire the surface morphology. This finding is of significant importance for the possible utilization of electropolymerization to thereby provide uniform, thin and adherent coating of medical devices.

Thus, according to a preferred embodiment of the present invention, the conductive surface is modified by attaching thereto an organic substance that forms SAMs on the surface, whereby the SAMs are selected so as to allow the performance of an electropolymerization thereon. Thus, the preparation of the electropolymerized polymer onto the modified surface is enabled by attaching a thin layer of the organic substance, which allows electron transfer therethrough.

Exemplary organic substances that can be used for that purpose include the fatty acids and the organosilanes described hereinabove. Thus, fatty acids having 3-30 carbon atoms and organosilanes having a hydrocarbon residue of 1-10 carbon atoms were found suitable for use in this context. Alternatively, the fatty acids or the hydrocarbon residue of the organosilane can by itself be substituted and preferably terminated by the an electropolymerizable monomer, such that the SAMs deposition and the electropolymerization are simultaneously effected with a single organic substance.

Representative examples of electropolymerized polymers that are usable in the context of this embodiment of the present invention include, without limitation, polypyrroles, polythiophenes, poly-p-phenylenes, poly-p-phenylene sulfides, polyanilines, poly(2,5-thienylene)s, fluoroaluminums, fluorogalliums, phtalocyanines, and any combination thereof, whereby the polymers can be used as is or as derivatives thereof in which the backbone unit is substituted by various substances that may provide the surface with the desired characteristics, e.g., polymers, hydrocarbons, carboxylates, amines and the like.

Representative examples of polypyrrole derivatives and the preparation of the corresponding monomers are described, for example, in WO 01/39813, which is incorporated by reference as if fully set forth herein, and in the above-mentioned U.S. patent application. Representative examples of pyrrole derivatives that can be used for attaching an electropolymerizable substituted polypyrrole to a modified surface, according to preferred embodiments of present invention, are also described in the Examples section that follows.

Thus, articles-of-manufacture according to the present invention modified surfaces which enable the attachment of active substances thereto. In one embodiment, the surfaces can be modified either electrochemically or non-electrochemically and preferably involve the attachment of an organic substance thereon, which includes functional moieties for attaching the active substance. Further preferably, such an organic substance forms SAMs on the surface.

In another embodiment, the surface is modified such that the active substance is directly attached, electrochemically, to the surface.

According to another aspect of the present invention, there is provided a process of preparing the articles-of-manufacture described hereinabove. The process is typically effected by electrochemically modifying a conductive surface of an object to thereby functionalize the surface by functional moieties, as is described hereinabove, and contacting the modified surface with an active substance, as described hereinabove, so as to allow interaction between the functional groups onto the surface and the active substance. Representative examples of such interactions are described in the Examples section that follows.

Optionally, the process may further comprise, subsequent to or concomitant with modifying the surface, electrochemically attaching the active substance to the modified surface.

Alternatively, the process is effected by directly attaching an active substance to the surface, by electrochemical means, as described hereinabove and is further exemplified in the Examples section that follows.

The versatile methodologies described herein for the preparation and provision of conductive surfaces that have active substances attached thereto can be used to provide tailored, well-defined coatings on metallic surfaces and are particularly advantageous for providing implantable devices with improved mechanical, physical, chemical and therapeutic characteristics, as is exemplified and detailed in the Examples section that follows.

The present invention therefore provides various articles-of-manufacture that can be prepared by controlled, yet versatile, processes, resulting in objects coated by various active beneficial active substances, whereby the coatings are characterized by enhanced adherence, enhanced density of the active substance and improved surface characteristics, as compared with the presently known coatings.

Using the processes above, the active substance may be directly or indirectly attached to surface. When indirectly attached, the active substance is preferably attached to a SAM deposited onto the surface. Bioactive agents can also be directly or indirectly attached to the surface and can be further coated by one or more films, through which they can diffuse in a controlled manner.

When these articles-of-manufacture are coated implantable devices they can be beneficially used in the treatment of conditions in which implanting a medical device, and particularly such a device loaded with bioactive agents, is beneficial.

Such conditions include, for example, cardiovascular diseases such as, but not limited to, atherosclerosis, thrombosis, stenosis, restenosis, and in-tent stenosis, cardiologic diseases, peripheral vascular diseases, orthopedic conditions, proliferative diseases, infectious diseases, transplantation-related diseases, degenerative diseases, cerebrovascular diseases, gastrointestinal diseases, hepatic diseases, neurological diseases, autoimmune diseases, and implant-related diseases.

As is described in detail in the U.S. patent application, entitled “Electropolymerizable monomers and polymeric coatings on implantable devices obtained therefrom”, mentioned hereinabove, a special system which allows an efficient preparation of the coated medical devices described herein was designed.

The system comprises, in operative arrangement, at least one holding device for holding the medical device, a conveyer, and a first and a second bath arranged along the conveyer, wherein the conveyer is designed and constructed to convey the at least one holding device such that the at least one holding device is placed within each of the first and second baths for a predetermined time period and in a predetermined order, and further wherein the first bath is a modification bath and the second bath is an active substance solution bath. Thus, a medical device can be modified, as described above, while being treated in a first bath and conveyed to a second bath, in which the active substance is attached to the surface. In cases where the active substance is directly electrochemically attached to the surface, the first bath and the second bath are included in one bath.

Alternatively, the modification bath comprises an organic substance having a functional moiety capable of interacting with the active substance, as described hereinabove, which is deposited onto the surface, either electrochemically or non-electrochemically.

The modified surface is then conveyed to the second bath, for attaching thereon the active substances. In cases where the active substance is an electropolymerized polymer, the second bath is an electropolymerization bath.

According to still further features in the described preferred embodiments the electropolymerization bath comprises at least one electrode structure, mounted on a base of the electropolymerization bath and connected to an external power source.

The system can be used for further conveying the medical devices through other baths, including, for example, pre-treatment baths, where the device surface is treated prior to modification (as is exemplified in the Examples section that follows), washing and rinsing baths, where residual reactants are removed, additional electropolymerization baths and chemical polymerization baths, depending on the methodology and active substances used for coating the device.

The additional baths are arranged along the conveyer, wherein the conveyer is designed and constructed to place the holding device within the baths for a predetermined time period.

The system preferably i further comprises a cartridge having a cartridge body adapted for enabling the at least one holding device to be mounted onto the cartridge body.

The holding device itself comprises a perforated encapsulation, adapted to receive the at least one medical device, and at least two cups adapted for enabling electrode structures to engage with the perforated encapsulation hence to generate an electric field within the perforated encapsulation.

The perforated encapsulation is preferably designed and constructed to allow fluids and chemicals to flow therethrough.

The conveyer is preferably operable to mount the at least one holding device on the at least one electrode structure, thereby to engage the at least one electrode structure with a first side of the perforated encapsulation.

The system may further comprises an arm carrying at least one electrode structure and operable to engage the at least one electrode structure with a second side of the perforated encapsulation.

Exemplary holding devices, cartridges and systems according to this aspect of the present invention, which are designed to provide electrocoated stents are presented in FIGS. 21-23.

FIG. 21 illustrates a device 10 for holding a stent assembly 12 while being coated, according to a preferred embodiment of the present invention. Holding device 10 comprises a perforated encapsulation 14 which receives stent assembly 12. Assembly 12 is shown in FIG. 21 as an expandable tubular supporting element 16 prior to its coating. Preferably, but not obligatorily, encapsulation 14 has a tubular (e.g., cylindrical shape). Device 10 preferably holds stent assembly 12 throughout the entire treatment of assembly 12. Thus, device 10 can hold assembly 12 while being treated in, for example, a chemical treatment bath, an electrochemical treatment bath, an ultrasonic bath, a drying zone, a drug loading bath and the like.

Perforated encapsulation 14 comprises a plurality of holes 24 formed on its wall 26 so as to allow various chemicals solutions 30 to flow from the respective treatment bath, through wall 26 and into an inner volume 28 of encapsulation 14 thereby to interact with stent assembly 12 and/or supporting element 16. Additionally, holes 24 preferably allow chemicals solutions to flow out of inner volume 28, for example when device 10 is pulled out of the respective treatment bath.

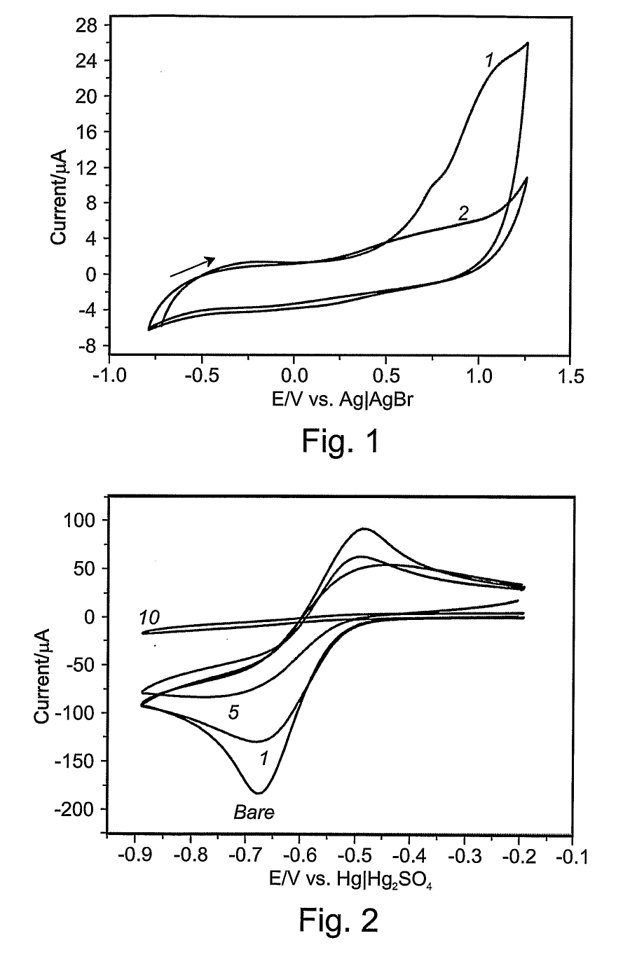

Device 10 further comprises two or more cups 18 covering a first end 20 and a second end 22 of encapsulation 14. Cup 18 can be made of, e.g., stainless steel. According to a preferred embodiment of the present invention cups 18 are adapted for enabling various electrode structures, designated in FIG. 1 by numerals 31 and 32, to engage with encapsulation 14. This embodiment is particularly useful when assembly 12 is subjected to electrochemical polymerization. Thus, a reference electrode can be inserted from one side and a counter electrode can be inserted from the opposite side. Additionally, a working electrode can be positioned near, say, a few millimeters apart from cup 18 such that, when the electrodes are connected to a power source (not shown), for example, via communication lines 36, an electric field is generated and redox reaction is driven on a working electrode 40. A polymerization process is thus initiated within volume 28 and member 16 is coated by the polymer film.

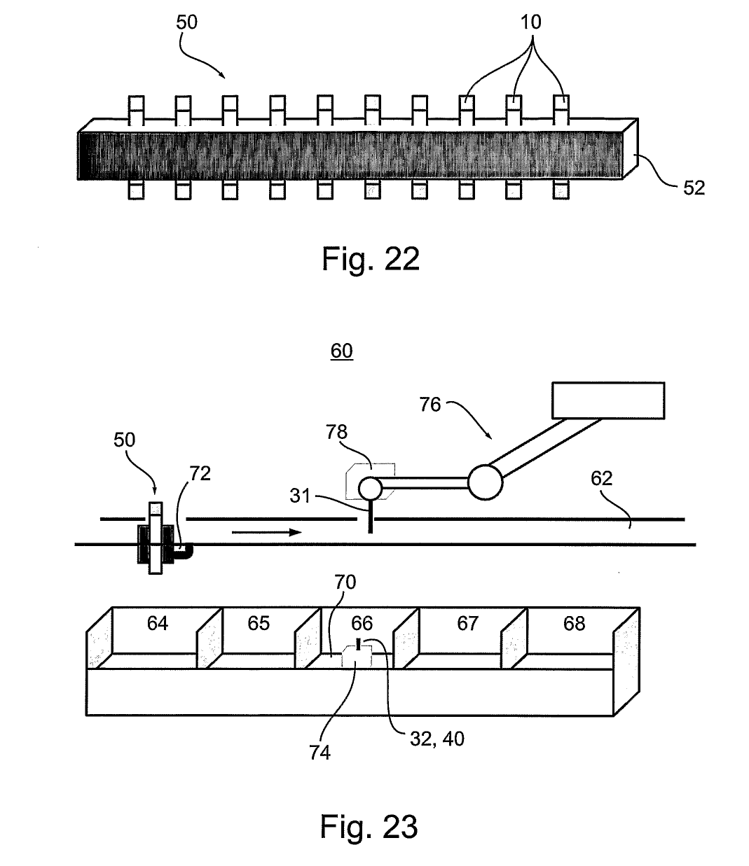

Several holding devices can be employed for coating several stent assemblies simultaneously. FIG. 22 is a schematic illustration of a cartridge 50 of holding devices. The principles and operations of each of the holding devices on cartridge 50 is similar to the principles and operations of device 10 as further detailed hereinabove. Cartridge 50 serves for placing several holding devices together in the treatment baths. In the exemplified configuration of FIG. 2 cartridge 50 holds 10 devices, but this need not necessarily be the case, and any number of holding devices can be mounted on a body 52 of cartridge 50. The body of the cartridge 50 is preferably designed to be mounted on a conveyer that places cartridge 50 in the treatment bathes as further detailed hereinbelow.

Reference is now made to FIG. 23 which is a schematic illustration of a system 60 for coating one or more stent assemblies, according to a preferred embodiment of the present invention. System 60 preferably comprises, in operative arrangement, one or more holding devices (e.g., device 10). When several holding devices are used, the devices are preferably mounted on a cartridge, for example, cartridge 50.

System 60 further comprises a conveyer 62 and a plurality of treating baths arranged along conveyer 62. In the representative example shown in FIG. 23, system 60 comprises five treating baths designated 64, 65, 66, 67 and 68. Thus, for example, bath 64 can be used as a pretreatment bath in which the stent assembly is subjected to chemical and mechanical treatments so as to prepare the stent assembly to a uniform and adherent coating. Bath 65 can be used for washing, bath 66 can be used for electrochemical polymerization, bath 67 can be used for cleaning and bath 68 can be used for drug loading. Other baths or treatment zones are also contemplated.

Conveyer 62 conveys the holding device(s) such that the device is placed within each treating baths in a predetermined order. Thus, for example, in the exemplified embodiment of FIG. 23, conveyer 62 places the device first in bath 64, then in bath 65 etc. Additionally, conveyer 62 controls the time period at which the device spends in each bath. This can be achieved by designing conveyer 62 to pull the device from the respective bath after a predetermined time period and place it in the next bath in line. Conveyer 62 is preferably manufactured with a lever 72 or any other mechanism for placing the device in the baths before treatment and pulling it out thereafter.

According to a preferred embodiment of the present invention the electrochemical polymerization bath comprises electrode structures (e.g., counter electrode 32 and working electrode 40) mounted on base 70 thus forming a lower electrochemical polymerization unit. The electrode structures preferably protrude out of an isolating material 74 (see also FIG. 1) and connected to a power source (not shown). In operation, conveyer 62 mounts the holding device on the electrode structure(s), which in turn engage with the one side of the device. System 60 can also comprise an arm 76 carrying one or more electrode structure (e.g., reference electrode structure 31), which preferably protrudes out of an isolating material 78. Arm 76 and electrode 31 thus form an upper electrochemical polymerization unit.

Once the holding device is mounted on electrodes 32 and/or 40, arm 76 causes electrode 31 to engage with the other (upper in the present embodiment) side of the holding device. Being in electrical communication with the electrodes, the stent assembly in the holding device can be subjected to the electrochemical polymerization as known in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Experimental Methods

Materials:

Decanoic acid (DA, 99+ %), myristic acid (MA, 99.5%), stearic acid (SA, 99+ %), tetrabutylammoniumtetrafluoroborate (TBATFB, 99%), hexaamineruthenium(III) trichloride (Ru(NH₃)₆Cl₃, 98%), NaNO₃ (99.9%) and NaCl (99.5%) were purchased from Aldrich. Acetonitrile (ACN, >99.8%) was obtained from J. T Baker. Palmitic acid (PA, 99%) was obtained from BDH Technologies (England). Pyrrole (99%) was obtained from Sigma-Aldrich and was freshly purified by alumina column chromatography before use. Alumina and silica gel for column chromatography were purchased from Merck, Germany. Organosilanes were purchased from Sigma-Aldrich and Merck.

316L Stainless steel plates and rods (also referred to herein collectively as electrodes) were purchased from Mashaf Co. (Jerusalem, Israel). The stainless steel plates (9×40 mm) were used for infrared spectroscopy and contact angle measurements while the rods were applied for electrochemical measurements. The stainless steel rod (3 mm diameter) was embedded in a Teflon sheath exposing only a disc, which served as the electrode surface.

Instrumentation:

Electrochemistry: Electrochemical measurements were conducted with an AUTOLAB PGSTAT10 potentiostat (EcoChemie, Utrecht, The Netherlands) and BAS-100B/W electrochemical analyzer (Bioanalytical Systems, Lafayette, Ind.), using a single compartment three electrode glass cell. The reference electrode was either a saturated Hg|Hg₂SO₄|K₂SO₄₍ₛₐₜ₎ electrode when aqueous solutions were employed or an Ag|AgBr wire that was used in organic media. The latter has a potential of 0.448 V vs. ferrocene-ferrocenium (Fc/Fc⁺) [26]. A 6 mm diameter graphite rod was used as an auxiliary electrode.

IR spectra: Fourier transform infrared (FTIR) spectra were recorded using an Equinox 55 (Bruker) spectrometer, at a resolution of 2 cm⁻¹ equipped with a nitrogen-cooled MCT detector. Normally, 1500 scans of the sample were collected versus a reference which was a bare stainless steel surface.

Contact angles: Contact angles were measured using a Ramé-Hart model 100 contact angle goniometer. Advancing and receding contact angles were determined by adding and withdrawing fixed amount of deionized water to and from the drop, respectively. This measurement was repeated three times for each sample, and the average values are reported.

XPS measurements: X-ray photoelectron spectra (XPS) were recorded using an Axis Ultra spectrometer (Kratos), and MgKα radiation of 1486.71 eV. Data were collected and analyzed by vision processing program.

SEM measurements: The surface morphologies of the unmodified and modified electrodes were measured by high resolution scanning electron microscopy (HR SEM) using a sirion scanning microscope (FEI Company, Holand) equipped with shottky type field emission source at 10 kV accelerating voltage. Samples were washed with acetonitrile and dried at room temperature before subjected to analysis.

All aqueous solutions were prepared from deionized water (Mili-Q, Milipore).

Experimental Methods:

Modification of stainless steel surfaces by fatty acids: Stainless steel rods were polished first with 240, 600 and 2000 grit emery paper (Buehler), followed by fine polishing by alumina paste (1 and 0.05 μm) on a microcloth polishing pad. Stainless steel plates were received polished from the supplier and were treated only with 2000 grit emery paper. The electrodes were thereafter washed with acetonitrile, sonicated (for about 15 minutes) in acetonitrile and dried under a stream of nitrogen at room temperature prior to the modification. The clean electrodes were immersed in a de-aerated modification solution containing 0.1 mM long-chain carboxylic acid (DA, MA, PA or SA) and 0.1 M TBATFB in acetonitrile at room temperature. A potential sweep ranging from −0.8 V to 1.2 V vs. Ag|AgBr of 10 cycles was typically applied (unless otherwise indicated). The modified surfaces were rinsed with pure acetonitrile and dried with a gentle stream of nitrogen.

The Ag|AgBr reference electrode was prepared by sweeping the potential of a polished silver wire electrode in 50% HBr solution from −0.3 V to 1 V vs. Ag|AgCl and scan rate of 10 mV·sec⁻². The potential was held at 1 V for 2 minutes and the electrode was thereafter pulled out from the solution, under potential, washed with water and dried in air. The potential of the resulted Ag|AgBr wire was frequently checked versus Fc/Fc⁺ [26]. Graphite rod was used as the auxiliary electrode.

Using a similar procedure, stainless steel surfaces were electrochemically modified by the following fatty acids substituted by various functional groups: 12-aminododecanoic acid, 12-hydroxydodecanoic acid, 10-hydroxydevanoic acid, and decandioic acid. In a representative example, the modification solution contained 0.1 M TBATFB in 10 ml ACN, 50 μL of 1 M HClO₄ solution and 1 mM 12-aminododecanoic acid. HClO₄ was added to increase the solubility of the fatty acid in the modification solution. The solution was de-aerated under a nitrogen stream for about 10 min before the modification process was conducted.

A 316 L stainless steel plate was further polished prior to modification, using 2000 grit emery paper (Buehler), pre-treated as described hereinabove and immersed in the modification solution. A potential sweep between −0.8 to 1.25 V vs. Ag|AgBr was typically applied (5 cycles unless otherwise mentioned) with scan rate of 100 mV·sec⁻¹. The modified surfaces were rinsed with pure ACN and dried with a gentle stream of nitrogen.

Capacitance: Double-layer capacity, C_{dl}, was measured in a 10 ml de-aerated aqueous solution of 0.1 M NaNO₃. The electrode was equilibrated for 10 minutes, and an ac voltage of 7 mV peak-to-peak and 320 Hz was superimposed thereafter on the dc potential (0-0.3 V versus Hg|Hg₂SO₄|K₂SO₄₍ₛₐₜ₎). The real and imaginary parts of the ac current were detected using an EcoChemie potentiostat equipped with a frequency analyzer (FRA). All measurements were performed at room temperature (21±2° C.).

Electropolymerization: Electropolymerization of pyrrole or derivatives thereof was conducted using cyclic voltammetry (CV) of stainless steel plate (40×9-5 mm²), as the working electrode, immersed in a polymerization solution containing 0.1 M distilled pyrrole monomer and 0.1 M tetrabutylammonium tetrafluoroborate (TBATFB) in acetonitrile. Ag|AgBr was used as the reference electrode while graphite rod was used as the auxiliary electrode.

Electropolymerization was performed using a bare stainless steel electrode, or a stainless steel electrode pre-treated with a long chain carboxylic acid, as described hereinabove. Alternatively, the electropolymerization was performed using a polymerization solution as described above, further containing 0.1 mM of a long chain carboxylic acid.

Adhesion measurements: The adhesion of a polymer film to a stainless steel plate was estimated using cross-cut tape test according to D-3359-02 ASTM standard, test method B. The film coating was cut into small squares (about 5×5 mm² each), the tape (Permacel 99, Permacel, New-Jersey) was stack onto the coating and was then striped off. The ratio between the number of adherent film squares remaining on the stainless steel plate and total number of squares was determined as the adherence factor.

Attachment of nanoparticles to stainless steel surfaces: A modification solution containing a suspension of PLA nanoparticles in 0.1 M TBATFB in 10 ml ACN suspension containing PLA nanoparticles was sonicated and de-aerated under nitrogen stream for about 10 minutes. A 316L stainless steel plate, treated as described above, were placed in the modification solution and a potential sweep between −0.8 to 1.25 V vs. Ag|AgBr was typically applied (10 cycles unless otherwise mentioned) with scan rate of 100 mV·sec⁻¹. The modified surfaces were rinsed with pure ACN and dried with a gentle stream of nitrogen.

Alternatively, a modification solution containing 0.1 M OxA in 9 ml pure H₂O and about 1 ml of poly(lactide co glycidol) particles suspension, and 0.01 M of pyrrole monomer was prepared. The solution was de-aerated under nitrogen stream for about 10 minutes before the modification process was conducted.

A 316L stainless steel plate was pre-treated as described above and a potential sweep between −0.8 to ˜1.3 V vs. Hg|Hg₂SO₄|KCl(sat.) was typically applied (10 cycles unless otherwise mentioned) with scan rate of 100 mV·sec⁻¹. The modified surfaces were rinsed with pure H₂O and dried with a gentle stream of nitrogen.

### Experimental Results

### Example 1

### Electrochemically-Induced Formation and Characterization of Fatty Acid Self-Assembled Monolayers on Stainless Steel Surfaces

Electrochemistry Measurements:

The formation of self-assembled monolayers (SAMs) on reactive metals, such as stainless steel, is considered as a non-trivial process, which is typically affected by the interactions between the amphiphilic moiety and the native oxide layer. The latter affects significantly the adhesive properties of the surface towards hydrophobic molecules. The formation of SAMs on stainless steel surfaces is highly advantageous as it provides for increased surface adhesion of organic biocompatible substances that can be attached to the distal end of the SAMs. One appealing approach for attaching such substances is through electropolymerization. Therefore, one of the most important prerequisite for the oxide layer is a controlled thickness thereof, which will enable electron transfer therethrough. Various methods have been described in the art for growing a controlled oxide layer, including chemical treatment, thermal treatment and electrochemical oxidation [27-31]. The formation of SAMs under potential control, where a gold surface was oxidized prior to the self-assembly process has also been described [32-34].

It was now surprisingly found that the formation of a well-ordered SAM of long-chain carboxylic acids on 316L stainless steel is efficiently performed by sweeping the potential of the surface in the presence of an acid-containing solution, as is generally described in the methods section above.

The cyclic voltammetry (CV) of a stainless steel electrode in an acetonitrile (ACN) solution containing 0.1 mM decanoic acid is shown in FIG. 1. In FIG. 1, the first two cycles are shown, where an anodic irreversible wave is clearly observed only in the first scan. A similar electrochemical behavior is seen in the absence of the acid, suggesting that the anodic wave results from the oxidation of the surface. It should be noted that subsequent cycles overlapped with the second scan. Furthermore, the oxidation wave was not detected when dry ACN was used.

The water contact angle of an electrode treated with decanoic acid as described hereinabove was compared with that of a stainless steel surface cycled without the presence of an acid and with that of a non-cycled stainless steel surface which was immersed in an acid-containing solution (denoted as bare). The obtained data is presented in Table 1. Interestingly, it was found that the water contact angle of an electrode treated with decanoic acid was significantly higher than in the case of cycling the stainless steel electrode in the absence of an acid. At the same time, the contact angle of an electrode, which was not cycled, however, immersed in the modification solution, did not change noticeably.

As is further shown in Table 1, the contact angle of a decanoic acid SAM, obtained by cycling the electrode 10 scans between −0.8 to 1.2 V vs. Ag|AgBr, was 87°. As it is known that increasing the chain length yields more highly ordered SAMs [20-25] almost regardless of the substrate, the advancing and receding water contact angles of SAMs made of various fatty acids that differ by their length have been measured. Thus, SAMs of decanoic acid (C₁₀), myristic acid (C₁₄) and palmitic acid (C₁₆) were formed on a stainless steel electrode under the same conditions. As is shown in Table 1, a clear trend in increasing the advancing contact angles was observed, indicating an enhancement in the SAM packing. The advancing contact angle values for myristic and palmitic acid SAMs are typical for a densely packed array of n-alkyl chains and are in line with those previously reported for comparable structures [16, 20, 23 and 25].

TABLE 1
Advancing and receding water contact angles measured on
316L stainless

| | Advancing | Receding | |
|---|---|---|---|
| | contact angle, | contact | |
| Monolayer Composition | deg ±1° | angle, deg ±1° | Hysteresis |
| Bare 316L SS | 65 | 47 | 18 |
| Bare 316L SS after | 59 | 45 | 19 |
| 10 cycles in 0.1M | | | |
| TBATFB/ACN | | | |
| Decanoic acid (10 cycles) | 87 | 70 | 17 |
| Myristic acid (10 cycles) | 97 | 82 | 15 |
| Palmitic acid (1 cycle) | 98 | 80 | 18 |
| Palmitic acid (5 cycles) | 102 | 90 | 12 |
| Palmitic acid (10 cycles) | 109 | 100 | 9 |

steel surface modified with n-alkanoic acids

The obtained data further indicated that the contact angle is affected also by the number of potential scans performed. As is shown in Table 1, the contact angle increased up to 109° upon cycling the stainless steel surface in the presence of palmitic acid. No further increase of the contact angle was observed when scanning the potential beyond ten cycles, suggesting that the layer reached its final organization. A control experiment in which a stainless steel electrode was cycled in the absence of an acid resulted in a contact angle of 59°, presumably attributed to the formation of an oxide layer. Evidently, the oxide layer formed in the absence of an acid is thicker than that formed in the course of assembling the organic monolayers. This issue is further addressed below with respect to the double layer capacity and the rate of electron transfer in these systems.

The contact angle data of SAMs on stainless steel, using alkanethiols and alkylamines, have been studied before and were found to be 104° and 105°, for hexadecaneamine and hexadecanethiol, respectively [5].

The ordering of the SAMs can be further evaluated by the hysteresis of the films, as it is known that when the ordering of the film increases the hysteresis drops. As is shown in Table 1, reduced values of hysteresis were obtained for the more ordered palmitic acid-treated surfaces, thereby indicating a consistent trend that stands in line with the expected results.

Electrochemistry Measurements in the Presence of a Redox Probe:

The formation of a thin film on a stainless steel surface was further evaluated and expressed by the cyclic voltammetry of the modified electrodes in the presence of a redox couple. Ru(NH₃)₆³⁺ was selected as the redox probe and evaluation was performed by first modifying the stainless steel electrode in an ACN solution containing 0.1 mM palmitic acid, as described above, and then transferring the modified electrode to an aqueous solution containing the redox probe. The effect of the number of potential cycles on the CV of Ru(NH₃)₆³⁺ is shown in FIG. 2 and the results clearly indicate that the reduction and oxidation waves of Ru(NH₃)₆³⁺ are reduced as the number of cycles increases, whereby after 10 cycles a complete blocking of the Ru(NH₃)₆³⁺ is attained. As is further shown in FIG. 2, in a control experiment (denoted as bare), in which a stainless steel electrode was swept for 10 cycles in an acid-free ACN solution, the number of cycles affected only the oxidation wave, yet, did not affect the electrochemical reversibility, as is further discussed hereinbelow. These results clearly indicate that blocking is due to the deposition of an acid SAM, which is induced by potential and not by oxide growth.

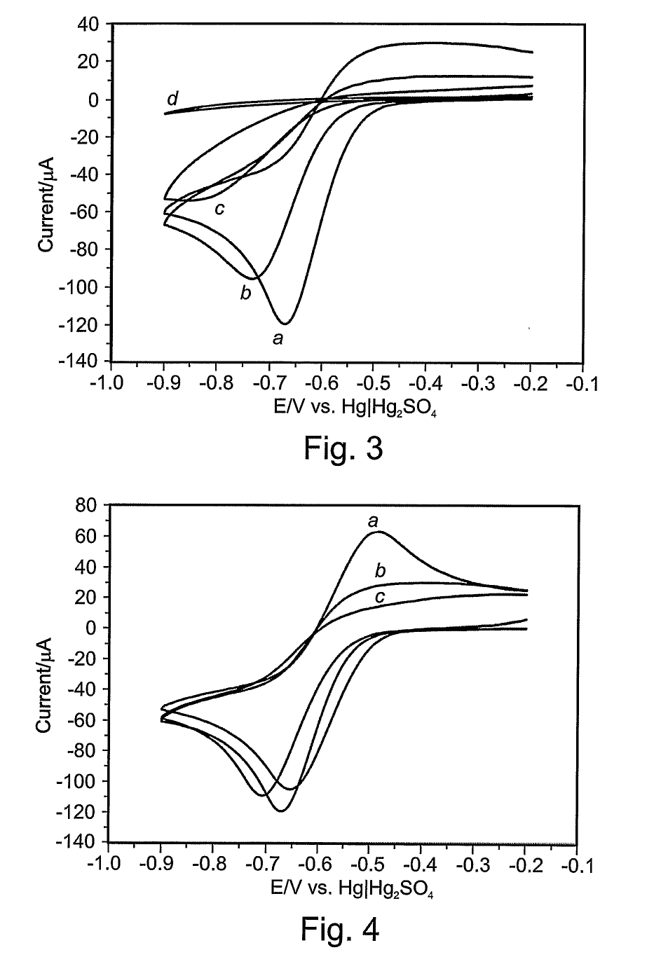

The blocking properties of stainless steel electrodes modified with decanoic acid, myristic acid and palmitic acid, as compared with a bare electrode (control), which was swept in an acid-free acetonitrile solution, using Ru(NH₃)₆³⁺ in an aqueous solution are presented in Table 2 below and in FIG. 3. The obtained data show that in electrodes having a decanoic acid film attached thereon, the reduction peak potential was only 63 mV more negative and its current was reduced by only 33%, as compared with the control bare stainless steel electrode. The relatively low effect of the decanoic acid film on the redox is not surprising, since, as is known in the art, a closed-packed self-assembled monolayer is typically formed when the chain length is C₁₁ and longer [20-21]. In the myristic acid modified electrode the peak potential was shifted by 175 mV to more negative potentials and the current was reduced by 66%, as compared with the control stainless steel electrode, whereas the electrode modified with palmitic acid showed almost complete blocking.

TABLE 2
Peak currents and potentials of the cathodic wave of Ru(NH₃)₆³⁺

| Stainless steel | | Peak potential/V |
|---|---|---|
| electrode | Peak current/μA | vs. Hg/Hg₂SO₄ |
| Bare electrode | 140.0 | 0.670 |
| Decanoic acid | 93.9 | 0.733 |
| Myristic acid | 47.0 | 0.845 |
| Palmitic acid | No peak | No peak |

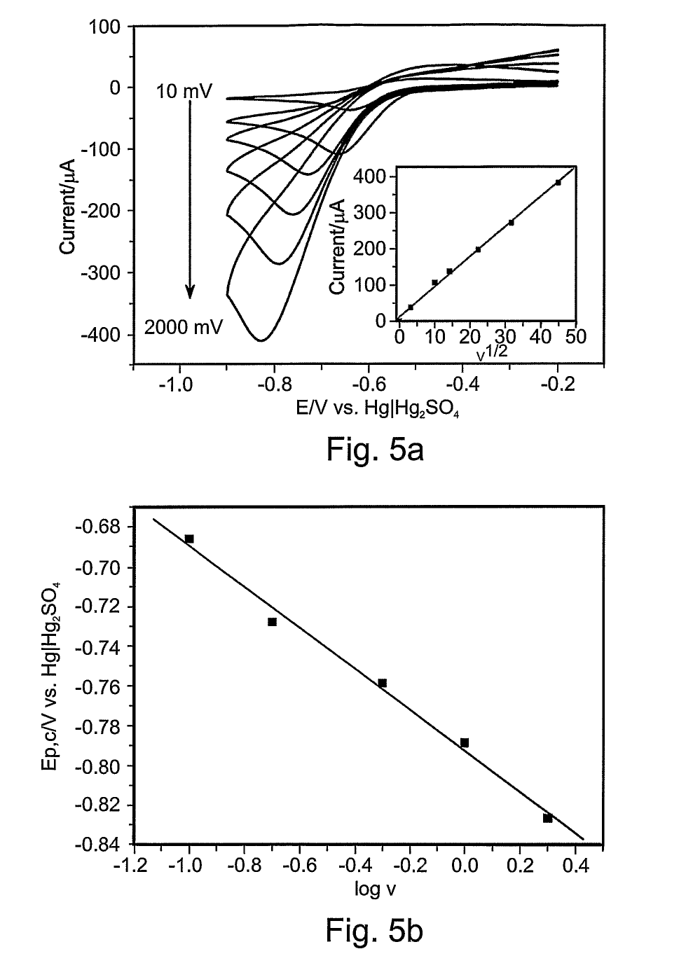

The kinetics of electron transfer using Ru(NH₃)₆³⁺ was further studied by comparing a bare stainless steel electrode and an electrode modified by decanoic acid. The obtained data is presented in FIGS. 4 and 5. FIG. 4 shows the effect of polishing and electrochemical cycling on the voltammetric behavior of Ru(NH₃)₆³⁺ using an unmodified (bare) stainless steel electrode and demonstrates that the freshly polished electrode exhibits a quasireversible behavior with a potential peak difference of 160 mV and a reduction-oxidation currents ratio close to unity. On the other hand, the CV of a freshly polished and electrochemically cycled modified electrode exhibits a chemically irreversible behavior where the oxidation wave is almost absent. A similar CV behavior was observed also with a polished electrode, which was left under ambient conditions for one day (FIG. 4), suggesting that the formation of an oxide layer has a pronounced effect on the oxidation of Ru(NH₃)₆²⁺.

FIGS. 5a-b show the effect of the scan rate on the CV of Ru(NH₃)₆³⁺ using a decanoic acid modified electrode. The measurements were carried out using IR compensation. As is shown in FIG. 5a, a linear dependence between the cathodic peak current and the square root of the scan rate was obtained, indicating that the reduction of the redox species is diffusion controlled.

Furthermore, the kinetic parameters, i.e., transfer coefficient (α) and standard heterogeneous rate constant (k⁰), were determined by plotting the peak potential as a function of the logarithm of the scan rate, v, according to Equation 1, where E_{c,p} is the cathodic peak potential and E⁰′, R, T and F have their usual meaning [35]:

$${E}_{c , p} = {E}^{{0}^{′}} - \frac{RT}{α F} [ 0.780 + ln ( \frac{{D}_{0}^{1 / 2}}{{k}^{0}} ) + {ln ( \frac{α Fv}{RT} )}^{1 / 2} ] \qquad ( Equation 1 )$$

As is shown in FIG. 5b a linear dependence is indeed obtained, allowing to extract α (0.3) and k_{monolayer}⁰ (1.6·10⁻³ cm·sec⁻¹) from the slope and intercept, respectively.

The same experiment and data treatment were performed with an electrode that was subjected to electrochemical cycling (two and ten cycles) in the absence of an acid. In this case, the transfer coefficient was ca. 0.5 in both cases and k_{bare}⁰ was ca. 4.0·10⁻³ and 2.3·10⁻³ cm·sec⁻¹ for two and ten cycles, respectively. The decrease of the rate of electron transfer upon increasing the number of potential scans is obviously due to the thickening of the oxide layer.

Following Amatore's approach [38], θ, which is the fractional coverage, can be derived by comparing the heterogeneous rate constant of bare and modified electrodes (Equation 2).

k_{monolayer}⁰=k_{bare}⁰(1−θ) (Equation 2)

Apparently, the fractional coverage depends to a large extent on the heterogeneous rate constant of a bare electrode. Introducing the values for the heterogeneous rate constants into Equation 2 yielded a fractional coverage of 0.6 and 0.3 for a bare electrode cycled for two and ten scans, respectively. It is conceivable that the oxide layer that is formed in the presence of an acid, as a result of cycling the stainless steel surface ten times, is significantly thinner than that formed in the absence of an acid. Nevertheless, θ is still lower than expected even when the rate constant of a bare electrode that was cycled only two scans was used. This is presumably attributed to the fact that some tunneling occurs across the decanoic acid film, an effect that is not included in Amatore's approach. In other words, electron transfer takes place not only in uncoated areas on the electrode, indicating an electron transfer through the acid layer. In acids having a longer chain, however, this treatment could not be applied due to complete blocking of electron transfer.

Capacitance Measurements:

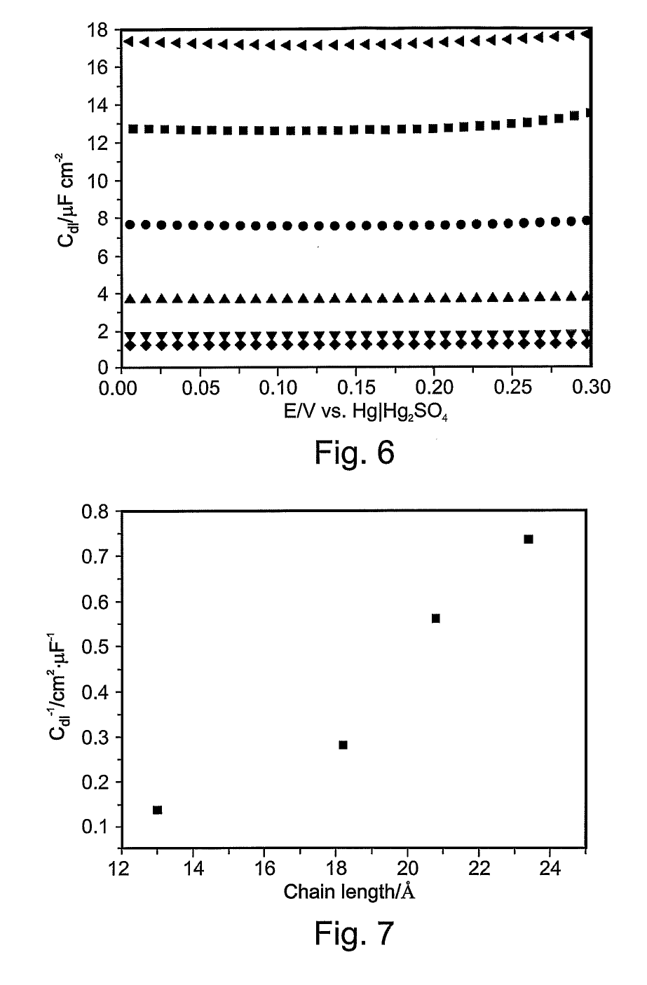

In order to verify that a monolayer rather than a multilayer was formed on the stainless still electrodes, the double layer capacity of different SAMs formed by deposition of four different long chain acids, decanoic acid, myristic acid, palmitic acid and stearic acid, was studied, as described hereinabove. The results are presented in FIG. 6 and show a dependence of the capacity on the potential of the modified and bare electrodes in 0.1 M NaNO₃. The capacity was measured by ACV between 0 to 0.3 V vs. Hg|Hg₂SO₄|K₂SO₄₍ₛₐₜ₎ and was found to be potential independent. The capacity of decanoic, myristic, palmitic and stearic acid SAMs were found to be 7.3, 3.57, 1.78 and 1.36 μF·cm⁻², respectively. These values are significantly lower than the capacity measured for a stainless steel electrode before (17.5 μF·cm⁻²) and after (13.0 μF·cm⁻²) it was cycled in the absence of an acid.

The fact that the capacity is not dependent on potential indicates that it can be described by the Helmholtz model [35-36], which is based on the assumption that the double layer behaves as a capacitor plate. In this case, the capacity is given by Equation 3, where ∈ is the dielectric constant, ∈₀ is the permittivity of free space, A is the area of the working electrode and d is the film thickness:

$${C}_{dl} = \frac{{ɛɛ}_{0} A}{d} \qquad ( Equation 3 )$$

The plotting the reciprocal capacity vs. the film thickness should therefore give a straight line. As is shown in FIG. 7, a linear relationship was indeed obtained between the reciprocal capacity and the length of the acid chains (assuming 1.3 Å per methylene, as discussed below). Linearity is increased when the decanoic acid is omitted, as is further supported by and in line with the electrochemical observations presented above.

Assuming a length of either 1.1 or 1.3 Å per methylene in an all-trans chain configuration, which corresponds to a tilt angle of 30° and 0° from the normal, respectively, gives a slope that varies between 1.0·10⁷-8.7·10⁶ cm·μF⁻¹. Since the

slope equals

$$\frac{1}{{ɛɛ}_{0}} ,$$

the dielectric constant of the layer can be estimated. A value of 1.13 and 1.30 was obtained for the dielectric constant of the layer for 30° and 0° tilt angles, respectively. These values are somewhat lower than the typical dielectric constants for pure aliphatic hydrocarbons and polyethylene, which are 2.0 and 2.3, respectively [36]. This can be explained by the fact that the interface is a series of two capacitors representing the oxide layer and the organic film. Nevertheless, the fact that a linear dependence is obtained suggests that the total capacity of the interface is governed primarily by the organic film.

Thickness Measurements:

It should be noted that ellipsometry studies performed in order to investigate the thickness of the formed monolayers were found to be very intricate due to a continuous oxidation process of the stainless steel surface. The bare stainless steel surface continuously oxidized while exposed to air, preventing the establishment of an adequate model of the oxide film that could be used for the organically modified stainless steel. This behavior was also reported by Tao [23, 25] and Nuzzo [37] when measuring the thickness of carboxylic acid monolayers on copper and aluminum oxides.

FTIR Measurements:

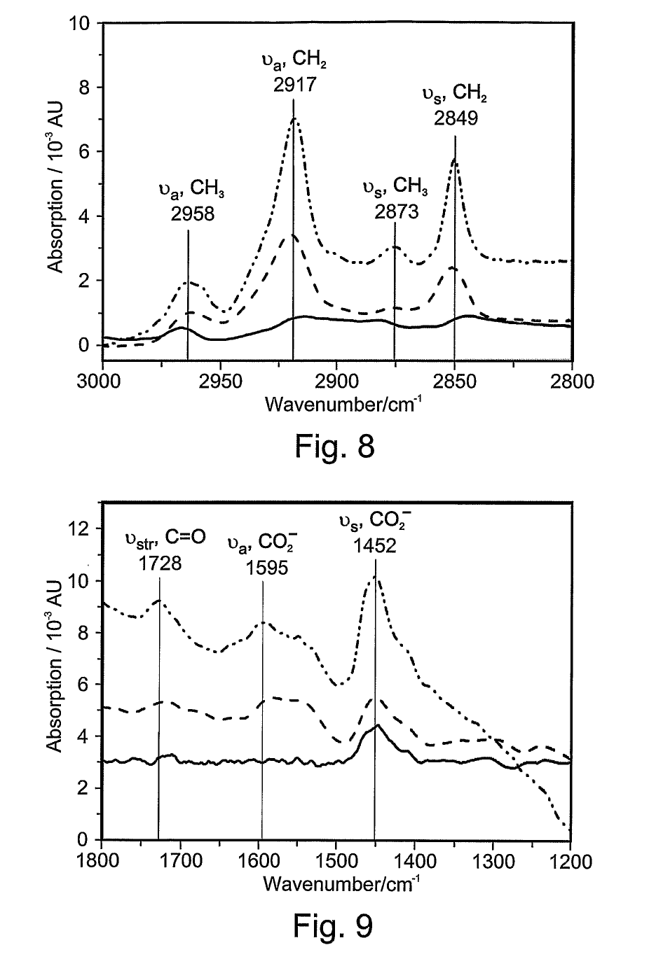

The acid films formed on 316L stainless steel were further characterized by reflection absorption Fourier transform infrared spectroscopy (RA-FTIR), in order to compare their structural features with n-alkanoic acid monolayers previously formed at other metal oxide surfaces [20-23]. FIG. 8 presents the C—H stretching region of the infrared spectra of decanoic, myristic and palmitic acid monolayers on 316L stainless steel substrates. The labeled peaks [21] represent the symmetric and asymmetric stretching modes of the methylene [(νₛ, CH₂) and (νₐ, CH₂)] and methyl [(νₛ, CH₃) and (νₐ, CH₃)] groups. Both the absolute intensities and peak locations indicate that the surface coverage and structure within the hydrocarbon chains of the myristic and palmitic acid films are comparable to those previously reported [20-25] for carboxylic acid monolayers on metal oxide surfaces. More specifically, the data presented for myristic (C₁₄) and palmitic (C₁₆) acid layers fit reasonably well with the monolayer assembly model in which the CH₃ group has its C—CH₃ rotation axis tipped closer to parallel to the stainless steel surface, where the alkyl chains axes are tilted g off the surface normal. The fact that the asymmetric and symmetric methylene vibration modes appear at 2918 and 2849 cm⁻¹, respectively, is evident of the highly ordered SAMs, which is similar to that found for carboxylic acid monolayers on copper [25].

Comparing the spectra of myristic and palmitic acid films reveals that their alignment is identical but the absorbance intensity of myristic acid is half than that of palmitic acid. The relative absorbance intensity can be attributed to three distinct contributions. The dominant contribution is due to difference in the tilt angle of the myristic and palmitic monolayers. As the tilt angle increases the component of the dipole moment perpendicular to the surface decreases, causing a decrease of the vibration intensity. Obviously, as the number of carbons in the hydrocarbon chain increases, the intensity will increase accordingly. This by itself cannot account for the difference between myristic (C₁₄) and palmitic (C₁₆) acid. The difference in surface coverage also affects the relative absorbance intensity. Since it was impossible to determine the surface coverage of myristic and palmitic acids (by electrochemical means), it was only speculated that the difference observed in the absorbance intensities between the two acids is a result of all these three contributions.

On the other hand, the spectrum of the shorter chain, decanoic acid (C₁₀), on oxidized stainless steel shows conformational disordering. These data is in accordance with the above-described interfacial properties of this layer and can be related to earlier studies on chemisorptions of carboxylic acid SAMs on other metal oxide surfaces. Highly oriented and closely packed monolayers were formed by amphiphiles bearing hydrocarbon chains longer than C_{11 [}20-23].

FIG. 9 presents the low-frequency region of an RA-FTIR spectrum of a palmitic acid monolayer on a stainless steel electrode, which relates to the head group stretching modes. The spectrum shows the symmetric (1452 cm⁻¹) and asymmetric (1595 cm⁻¹)—CO₂⁻ stretching vibrations, and a much weaker peak at 1728 cm⁻¹, corresponding to C═O stretching in the CO₂H moiety. These data are in conjunction with the lack of a C═O stretching mode at 1703 cm⁻¹, which is characteristic of a hydrogen-bonded carboxylic moiety, and indicate that the carboxylic head group undergoes partial dissociation to form a surface carboxylate species. The asymmetric —CO₂⁻ stretching mode is known to be dependent on the local environment and the nature of the ionic interaction with the substrate [20, 22]. Indeed, this signal is broadened and contains several overlapping peaks, indicating that the head group interacts via a number of different modes with the surface. Overall, the FTIR data obtained are similar to those reported for carboxylic acid SAMs on copper oxide [22], whereas the spectra of carboxylic acid monolayers on silver and alumina [20, 22] were quite different. The fact that a relatively weak peak of C═O is observed at 1728 cm⁻¹ suggests that indeed the head groups are adsorbed in several different orientation and local environments on the oxide surface of 316L stainless steel. It may be concluded that the majority of the SAM molecules are adsorbed on 316L stainless steel as carboxylate species, which are attached to the surface via ionic interactions, whereby a small fraction of the head groups does not undergo proton dissociation and is entrapped at the oxidelmonolayer interface as carboxylic species. The latter is presumably due to the fast deposition induced by applying an external potential.

XPS Measurements:

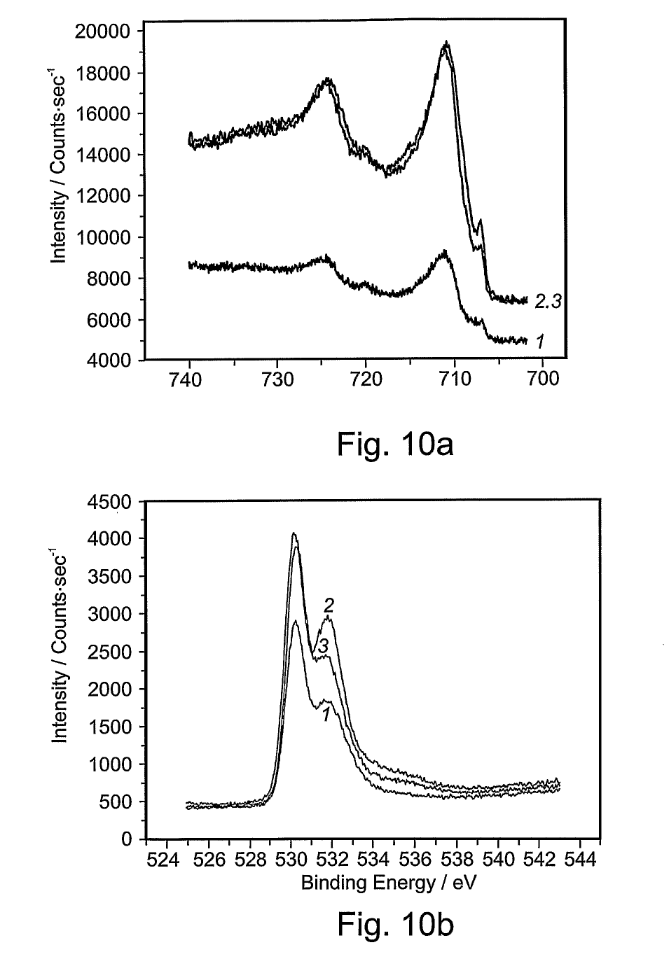

X-ray photoelectron spectroscopy (XPS) allows fine characterization of the different elements constituting the upper most layer of a substance. The XPS spectra of Fe2p_{3/2} and O1s of a palmitic acid modified stainless steel plate are presented in FIGS. 10a and 10b, respectively. As can be seen in FIG. 10a, the organic SAM attenuated the Fe2p_{3/2} signals. As can be seen in FIG. 10b, the electrochemical cycling enriched the metal/monolayer interface with —OH surface groups.

### Example 2

Formation of Organosilane Self-Assembled Monolayers on Stainless Steel Surfaces

Stainless steel surfaces having various organosilane SAMs were prepared as described below. Robust and uniform organosilane self-assembled monolayers were obtained. Since in some cases it is desired that the monolayers would be able to transform electrons during a following electropolymerization, preferably, organosilanes having an alkyl of 10 carbons or less are used. Following are the structural formulas of representative examples of organosilane derivatives:

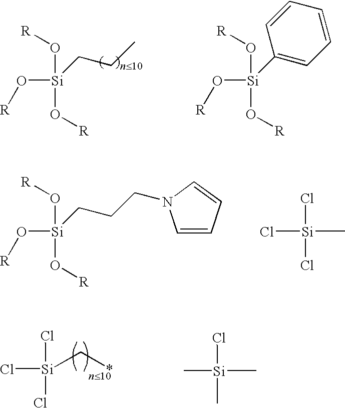

These therefore include, for example, SAMs formed from alkyl trialkoxysilne, aryl trialkoxysilane, alkyl trichlorisilane, trialkylchlorosilane and pyrroloalkyltrialkoxysilane. The latter can be used directly after or concomitant with the SAM deposition in the preparation of surfaces having a polypyrrole film attached thereon, via the SAMs.

Modification of Stainless Steel Surfaces by Organosilanes:

The monolayers deposition was carried out in two steps: (i) Stainless steel surface pretreatment, and (ii) SAM deposition.

Stainless steel samples were mechanically polished with D4000 grit emery paper, following by alumina paste (1 and 0.05 μm), and were then sonicated in an organic solvent for 15 minutes. All samples achieved a mirror-like finish. Finally samples were dried with a gentle stream of Nitrogen (N2) and kept in an inert atmosphere. Some standard cleaning methods may also be used to treat the SS surface, such as Oxygen plasma (Femto system) treatment or immersion in “piranha” solution prior to SAM deposition. The surface was optionally further treated with tetramethylortosilicate, so as to produce a silicon oxide anchoring layer, in order to enlarge the amount of hydroxyl groups on the SS surface. The thus treated surface was hydrolyzed prior to the formation of the SAMs, so as to produce the free hydroxyl groups.

The deposition was performed by simple immersion of the stainless steel samples in a diluted organosilane solution. In cases where halide organiosilanes were used, the deposition was performed under humide conditions and/or in the presence of water, so as to allow the coversion of the halide to hydroxy and thus to allow its attachment to the surface. The modified surfaces were thereafter cleaned of any excess materials, dried and kept in inert atmosphere.

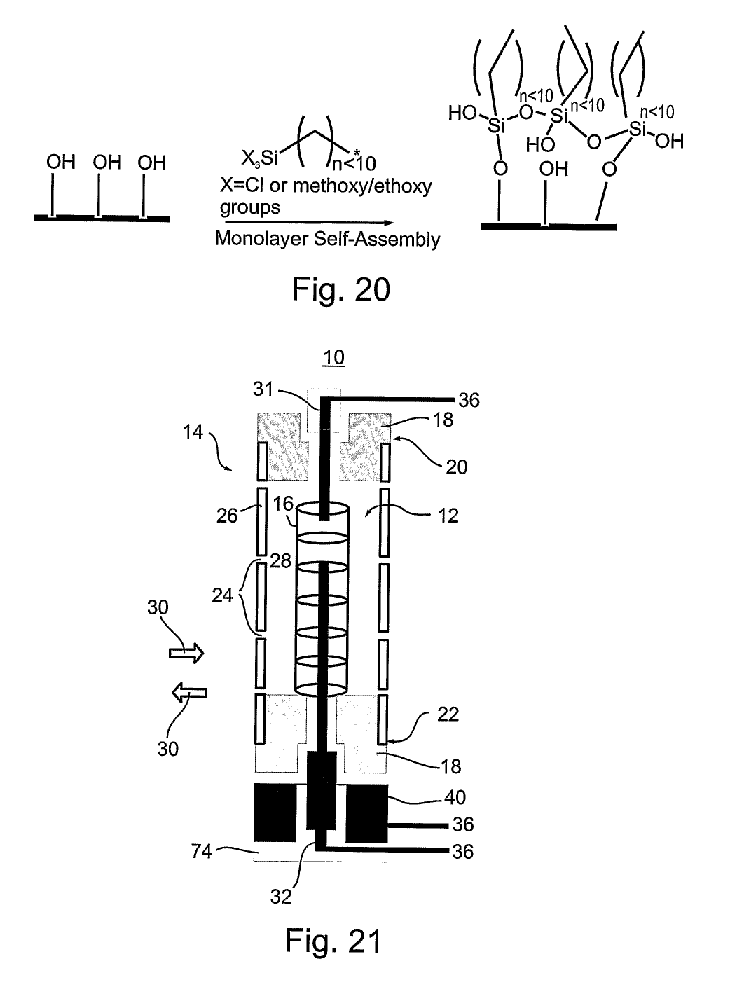

A schematic representation of the organosilane SAM deposition process is presented in FIG. 20.

### Example 3

### Electropolymerization of Pyrroles onto Modified Stainless Steel Surfaces

Electropolymerization of Pyrrole onto Stainless Steel Surfaces Having Fatty Acid Self-Assembled Monolayers Applied Thereon:

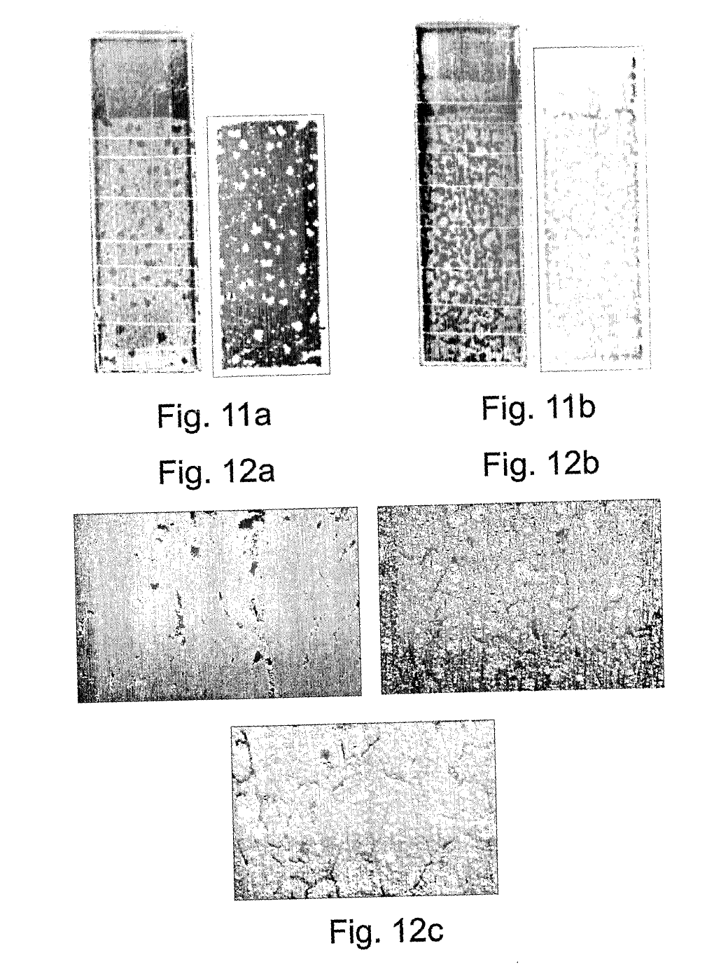

Electropolymerization of pyrrole and evaluation of the adhesion of the resulting film were performed as described above. The results are presented in FIGS. 11a-b. As can be seen in FIG. 11a, electropolymerization of pyrrole on a bare stainless steel plate resulted in less than 5% adhesion and the film was almost completely torn from the surface. However, when electropolymerization was conducted in a polymerization solution containing 1 mM decanoic acid, the adhesion increased to about 40%, indicating that incorporating a carboxylic acid increases the adhesion of the polymer coating on stainless steel. Moreover, as can be seen in FIG. 11b, the adhesion was further increased to more then 65% when the stainless steel plate was pre-treated with decanoic acid. The decanoic acid film remained on the stainless steel surface even after the second standard strip-off test.

The effect of decanoic acid SAM on the surface morphology was studied using scanning electron microscopy (SEM) as described hereinabove. As is shown in FIGS. 12a-c, polypyrrole films deposited over decanoic acid-modified stainless steel electrodes (FIG. 12c) have a smoother morphology as compared with that of the film deposited over bare stainless steel plates (FIG. 12b). These results suggest that the presence of the acid SAM on the surface creates an organic environment and allows the production of more nucleation sites for the growing of the organic polymer chains, relative to a bare surface. Same beneficial effects can be obtained by depositing organosilne SAMs, as described herein.

Thus, a simple and cost-effective method was demonstrated for the preparation of adherent and homogeneous polymer coating on 316L stainless steel surfaces. The low density of the decanoic acid SAM enables an electron transfer process and thus, improves the polymer-to-metal adhesion. The decanoic acid SAM serves as an interface between the metal surface and the polymer, while the metal surface effectively reacts with the carboxylate anions and the polypyrrole. The polypyrrole, on the other hand, is hydrophobic and interplays with the fatty chains of the monolayer. In addition, the SAM, as an adhesion promoting film, is molecularly dimensioned and thus the polymer film deposited thereon, having a thickness that can be controlled, acquires the surface morphology. This finding is of significant importance for the possible utilization of electropolymerization to thereby provide uniform, thin and adherent coating of medical devices.

Preparation and Electropolymerization of Pyrrole Derivatives onto Stainless Steel Surfaces Having Fatty Acid Self-Assembled Monolayers Applied Thereon:

Various pyrrole derivatives were prepared as electropolymerizable monomer units for electrocoating modified stainless surfaces. The pyrrole derivatives were designed to have functional groups that can serve to attach additional substances to the surface, whether bioactive substances and/or chemical substances that can improve the attachment of bioactive substances to the formed pyrrole film. Following are the structural formulas and syntheses of representative examples of pyrrole derivatives.

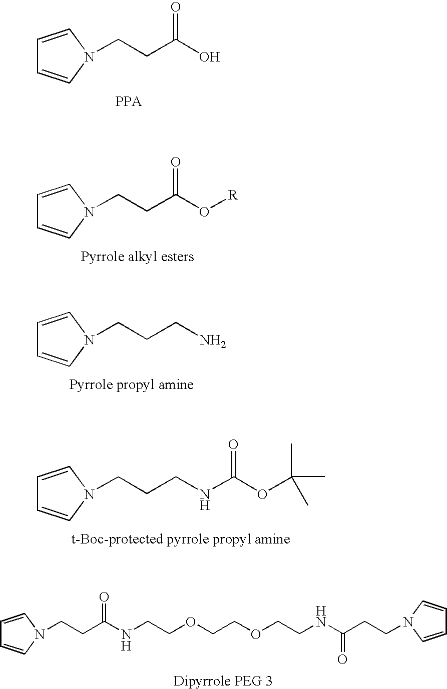

The following describes the preparation of a variety of electropolymerizable pyrrole monomers, derivatized by functional groups, which are suitable for use in the context of the present invention.

Preparation of Carboxylic Acid or Amino Containing Pyrrole Derivatives—General Procedure:

The preparation of carboxylic acid or amino containing pyrrole analogues was conducted based on known protocols by Yon-Hin et al, [Anal. Chem. 1993, 65, 2067-2071], unless otherwise indicated.

Preparation of N-(3-aminopropyl)-pyrrole (APP)—Route A: N-(2-cyanoethyl)pyrrole was reduced with LiAlH₄ in dry diethyl ether, using the general procedure described above, using N-(2-cyanoethyl)pyrrole (available from Aldrich Chemicals) as starting material. N-(3-aminopropyl)-pyrrole was synthesized by reduction of N-(2-cyanoethyl)pyrrole with LiAlH4 in dry diethyl ether in a 90% yield and was identified by H-NMR and IR (data not shown).

Preparation of N-(3-aminopropyl)-pyrrole (APP)—Route B: In an alternative synthetic route, APP was prepared as follows:

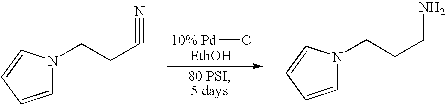

To 2-cyanoethyl pyrrole (10 grams, 83.3 mmol) dissolved in 50 ml methanol, 1 gram of 10% Pd—C were added and the vessel was connected to the hydrogenation system under 70 PSI for 4 days. The solids were precipitated off, the filtrate was collected and the volatiles were removed under reduced pressures. The obtained amine was purified on silica gel chromatography using 20-50% methanol in CHCl₃ as eluent, to afford N-(3-aminopropyl)-pyrrole in a 90% yield. The brownish viscous oil was characterized using NMR (data not shown) and ESI-MS.

ES-MS: m/z=122, 126, 153, 132, 339.

Preparation of N-(2-carboxyethyl)pyrrole (PPA): N-(2-cyanoethyl)pyrrole was hydrolyzed in aqueous KOH, according to general procedure mentioned above, as follows:

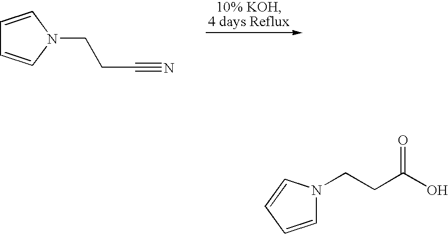

N-(2-Cyanoethy) pyrrole (10 ml, 83.23 mmol) was refluxed in a mixture of 20 grams KOH solution in 50 ml DDW and 10 ml ethanol for 4 days. Once the ammonia evolvement was ceased, the reaction mixture was allowed to cool to room temperature and the solution was acidified using concentrated hydrochloric acid until pH of about 4-5 was reached. The acid was extracted from the reaction mixture with 4×100 ml fractions of CH₂Cl₂. After drying on anhydrous sodium sulfate the organic solvents were removed to dryness under reduced pressure. The yellowish gum product N-(2-carboxyethyl)pyrrole, solidified after cooling and was obtained in a yield of 80% (melting point 58-59° C.).

¹H-NMR (DMSO-d₆): δ=6.749-6.735 (d, 1H), 5.964-5.948 (d, 1H), 4.103-4.058 (t, 2H, CH₂—), 2.661-2.614 (t, 2H, CH₂—) ppm.

MS (ES-MS): m/z (%)=164.6 (MW+Na+H⁺).

### Preparation of N-(2-Carboxyethyl) pyrrole-NHS(PPA-NHS)

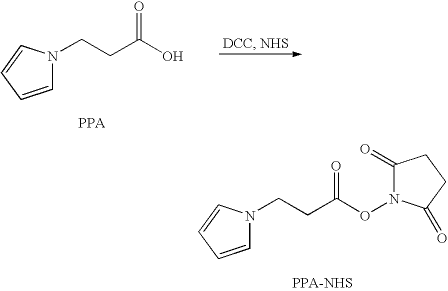

2-Carboxyethypyrrole (5 grams, 36 mmol) was dissolved in 70 ml ethyl acetate under calcium chloride tube. To the stirred solution 1.1 equivalent of dicyclohexyl carbodiimide (DCC) and N-hydroxysuccinamide (NHS) were added and stirred continuously. After a while, a white precipitate of DCU was formed. The mixture left to stand at room temperature for overnight, and the precipitate was filtered off and washed with two fractions of 50 ml ethyl acetate. The ethyl acetate fractions were collected and the solvents were removed under reduced pressure until dryness. The white colored residue was collected and stored at −5° C. until use. The product was identified by ¹H-NMR (data not shown).

Preparation of PPA-O-Peg-Oh: Pyrrolylation of Ho-Peg-Oh was established through an esterification process in toluene using isotropical reflux with p-toluene sulfonic acid (PTSA) catalysis, as follows:

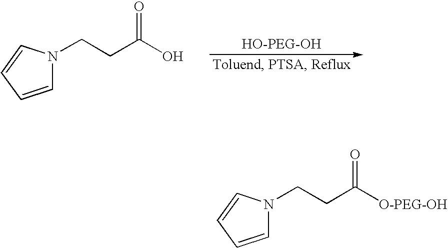

Using the procedure described above, equimolar amounts of PPA and PEG (MW=400) were dissolved in toluene in the presence of PTSA and the mixture was refluxed while distilling out the formed azeotrope, for 4 days. TLC has confirmed the formation of one major product and a residual amount of the starting material. The major product was identified by ¹H-NMR (data not shown).

### Preparation of Bis-Pyrrole-PEG220

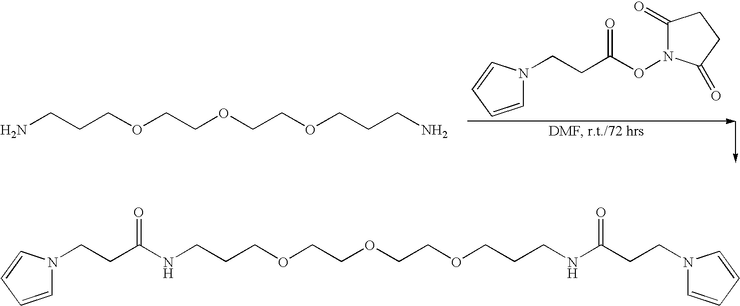

H₂N-PEG₂₂₀-NH₂ (1 gram, 4.54 mmol) was dissolved in 50 ml DMF. Then, PPA-NHS (2.14 grams, 9 mmol) dissolved in 20 ml DMF was added dropwise. The mixture was stirred at room temperature for 48 hours. Upon completion of the reaction the solvents were removed to dryness under reduced pressure. The bis-pyrrolylated residue was separated between 50 ml double distilled water (DDW) and CH₂Cl₂ and was extracted to 3×70 ml CH₂Cl₂. The organic fractions were dried on anhydrous sodium sulfate and the solvent was removed under reduced pressure. The residue was then purified on column chromatography and the final product was identified by ¹H-NMR (data not shown).

Preparation of Pyrrole Alkyl Esters: Pyrrole Alkyl Esters were Prepared by direct esterification of PPA. In brief, a solution of PPA in excess of alkyl alcohol was heated overnight at 70-80° C., in the presence of a catalytic amount of p-toluene sulfonic acid and magnesium sulphate. The solvent was thereafter evaporated, and the resulting ester derivative was extracted with a saturated sodium bicarbonate solution and ethyl acetate. The product was purified on a silica gel column using a mixture of dichloromethane and the alkyl alcohol as eluent. The product was characterized and identified by H¹ NMR, IR (data not shown).

Preparation of Pyrrole Propyl Amine: Pyrrole Propyl Amine was Synthesized by reduction of N-(2-cyanoethyl)pyrrole with LiAlH₄. In brief, to a suspension of 2.5 equivalents of LiAlH₄ in dry ether (90% of the total solvent volume), 1 equivalent of N-(2-cyanoethyl)pyrrole in dry ether (10% of the total solvent volume) was added, and the resulting mixture was refluxed overnight. After the reaction mixture was cooled down, the excess of LiAlH₄ was disactivated by small portions of DDW, 15% NaOH solution, and another portion of DDW. The mixture was then heated to 40° C. and stirred for 2 hours, filtered thereafter through a celite powder and the solvents were evaporated to give the product as yellow viscous oil. Pyrrole propyl amine was characterized by H¹ NMR, TNBS test and elemental analysis (data not shown).

Preparation of T-Boc-Protected Pyrrole Propyl Amine: T-Boc-Protected Pyrrole was prepared in order to improve the electropolymerization of pyrrole propylamine. In brief, pyrrole propyl amine (1 equivalent) was added to a methanol solution of Di-Boc. The reaction mixture was stirred under nitrogen atmosphere at 0° C. for two hours and was thereafter allowed to warm to room temperature. The reaction was proceeded overnight. The solvent, including the mono-Boc residue, was then evaporated to give the amine-protected product. The product was characterized by H¹ NMR (data not shown).

### Preparation of PPA-JEFAMINE2000-NH₂

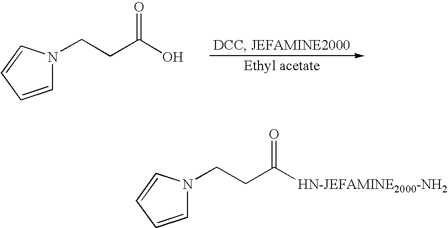

JEFFAMINE2000 (O-(2-aminopropyl)-O′-(2-methoxyethyl)-O′-(2′-methoxy ethyl)propylene glycol 2000, 10 grams, 5 mmol) was dissolved in 150 ml of ethyl acetate. While stirred, PPA (0.7 grams, 5 mmol) and DDC (1 gram, 7 mmol) were added thereto. The mixture was stirred at room temperature for 72 hours. Throughout this time a white DCU precipitate formed. The precipitate was filtered off and washed with two 20 ml fractions of ethyl acetate. The ethyl acetate fractions were collected and evaporated to dryness. The obtained yellowish gum was allowed to cool to room temperature and after a while solidified. The product was then purified by gel filtration and identified by ¹H-NMR (data not shown).

Preparation of Dipyrrole PEG 3: Dipyrrole PEG 3 was synthesized by simple amidation of PPA and jeffamine. In brief, PPA (1 equivalent) was dissolved in dimethyl formamide and the solution was cooled to 0° C. in an ice bath. DDC (50% excess) was added and the resulting mixture was stirred at 0° C. for one hour. Jeffamine (1 equivalent) was then added the reaction mixture was stirred for additional one hour at 0° C. and then overnight at room temperature. The mixture was treated with a small portion of acetic acid and water, stirred for 2 hours, and thereafter filtered. The precipitate was washed with dimethylformamide, the filtrate was evaporated to dryness and extracted with ethyl acetate, hydrochloric acid (0.1 M, twice), NaHCO₃ (twice) and NaCl saturated. The product solution was dried over magnesium sulphate and the solvent was evaporated, to yield the product. The product was characterized by H¹ NMR (data not shown).

Preparation of N-Alkylated Pyrroles—General Procedures:

In a typical reaction, pyrrole was first reacted with NaH, K or butyl lithium to obtain alkali pyrrole derivatives. These were reacted with equimolar amount of acyl halide or haloalkyl as previously described (E. P. Papandopoulos and N. F. Haidar, Tetrahedron Lett. 14, 1721-23, 1968; T. Schalkhammer et al. Sensors and Actuators B, 4, 273-281; S. Cosneir, Electrtoanalysis 1997, 9: 894-902 and references therein). Finally, the pyrrole alkali salt was conjugated with monobromo methoxy Polyethylene glycol (PEG) of various lengths (MW=200, 1000, 4,000 grams/mol, compounds 1, 2 and 3, respectively).

An alternative general procedure sodium hydride was used for in situ preparation of the pyrrolide anion, as follows:

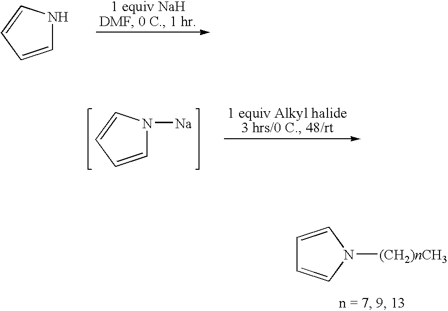

Thus, freshly distilled pyrrole (1 ml, 15 mmol) was dissolved in 30 ml of dry DMF under calcium chloride tube and the solution was cooled to 0° C. in ice cold bath. 1 equivalent of sodium hydride was added as an oil dispersion in fractions to the stirred solution. Immediately, gas evolution was noticed and the mixture was gently stirred for 60 minutes. To the cooling yellowish foam, an alkyl halide (1 equivalent, e.g., octyl iodide, docyl iodide, C₁₄-bromide) dissolved in 20 ml dry DMF, was added dropwise, and the mixture was stirred at 0° C. for additional 4 hours. Thereafter, the mixture was allowed to warm to room temperature, and was left for 48 hours. The DMF was removed to dryness under reduced pressure and the product was extracted from 100 ml DDW to 4×100 ml CH₂Cl₂. The organic fractions were collected and dried over anhydrous sodium sulfate. The organic solvent was then removed to give a brown oil. Purification was performed by distillation under vacuum at 180° C.

### Preparation of derivatized and analogs of 1,2-di(2-pyrrolyl)ethenes—general procedure

1,2-Di(2-pyrrolyl)ethenes and related compound were prepared according to Hinz et al. [Synthesis, 620-623 (1986)], as follows:

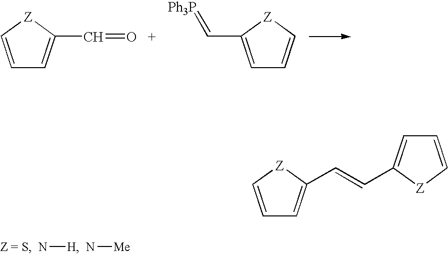

Thus, 1,2-Di(2-pyrrolyl)ethenes and related compounds were prepared via the Wittig reaction between commercially available 2-thiophen carboxyaldehyde or 2-(N-alkylpyrrole)-carboxyaldehyde and the corresponding methyl phosphonium salts (prepared via the Mannich reaction of unsubstituted pyrrole) in toluene (10 hours reflux under argon atmosphere). The overall yields were about 70%.

### Preparation of 1,1′-di-(2-thienyl or pyrrolyl)-2-alkyl ethylene—general procedure

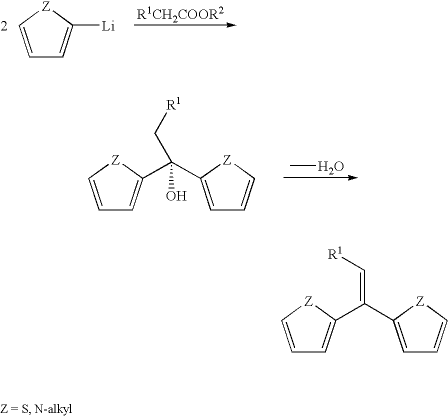

1,1′-Di-(2-thienyl)ethylene was prepared by reacting 2-acetylthiophe with the granger reagent of 2-bromothiophen in dry THF. The product was identified by 1H-NMR and EI-MS (data not shown).

The Pyrrole analogs were prepared in a similar manner, based on Ramanthan et al. [J. org. chem. 27 1216-9 (1962); and Heathcock et al. [J Heterocyclic chem. 6(1) 141-2 (1969)], via the lithiation of N-Alkylpyrrole in dry hexane or THF with TMEDA at room temperature, followed by disubstitution of the corresponding ester.

The conjugated product was easily obtained in dilute hydrochloric acid.

Further derivatization may be achieved via esterification of the hydroxyl with various carboxylic acids, using known procedures.

Coupling of thienyl, furanyl, and N-Alkyl pyrrole derivatives—general procedure: Coupling of the 2-lithium derivative of both thienyl and furanyl derivatives and N-Alkyl pyrrole was performed as follows:

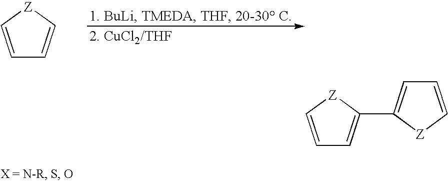

The various coupling products were easily obtained in relatively good yields (about 70%) using CuCl₂, although other reagent such as NiCl₂ can also be used, as proposed in the literature [chem. Ber 114 3674 (1981)].

Preparation of Electropolymerizable Thienyl and Pyrrolyl Monomers:

1,4-di(2-thienyl)-1,4-butandiol was prepared using Stetter reaction [Stetter, H; Angew chem. 88, 694-704 (1976)] according to Wynberg [Wynberg et al synthetic comm. 1 14(1) (1984)] in a 75-80% yield. 1,4-di(2-thienyl)-1,4-butandiol was then reacted with the corresponding amine to prepare the 2,5-di(2-thienyl)N-alkyl pyrrole via the Paal-Knore reaction [Cava et al Adv materials 5 547 (1993)], as follows:

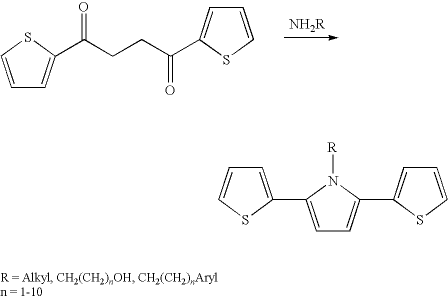

The N-alkylhydroxy derivative was conjugated to various carboxylic acid via esterification prior to polymerization.

### Preparation of 3-alkyl-(N-Methylpyrrole) derivatives

The preparation of 3-alkyl-(N-Methylpyrrole) derivatives is depicted as follows:

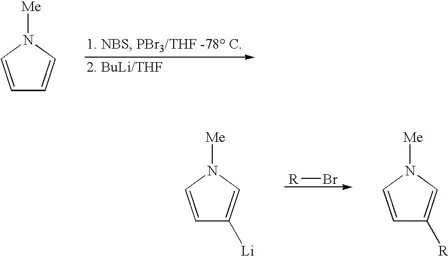

Alkyl pyrrole was selectively brominated with N-bromosuccinimide and PBr₃ in THF according to Dvorikova et al [Dvorikova et al. Synlett 7 1152-4 (2002)]), and was then reacted with BuLi in THF at −78° C. The product was obtained through a reaction with the alkyl halide.

Preparation of Thienyl and N-Alkyl Pyrrolyl Via Dilithiation:

The N-alkyl modified pyrrole was lithiated and the resulting 2-lithium pyrrole derivative was further reacted with 2,5-dibromothiophen.

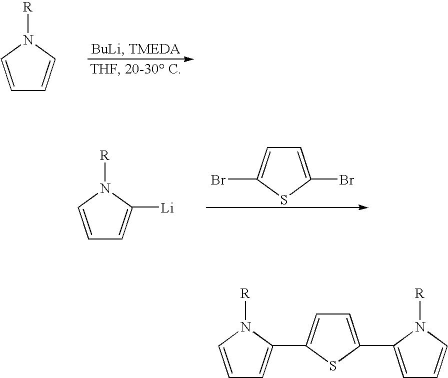

Preparation of Thienyl and Di(N-Alkyl) Pyrrolyl Dimethanol Oligomers:

The bis-pyrrole compound (obtained as described above) was lithiated and the resulting lithiated bis-pyrrole was reacted with an equimolar amount of the corresponding aldehyde, as follows:

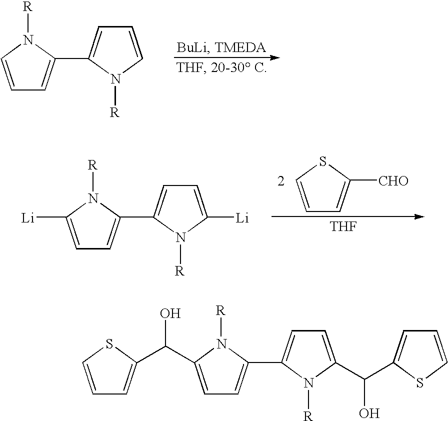

The reaction was carried out according to the procedure described in the literature for reactions of lithium derivatives with aldehyde and ketones in THF under inert conditions [Cava et al Adv materials 5 547 (1993)].

Similar furanyl, pyrrollyl and di(N-alkyl)pyrrole dimethanol oligomers were also prepared using the same process.

Preparation of 2-Alkyl Pyrrole Derivatives—General Procedure:

Terminal N-Alkyl pyrrole having alkyl and aryl groups in the alpha position were designed as terminators for the electrochemical polymerization and control of the molecular weight (MWD) of the polymer. These compounds were prepared as follows, based on the procedure described in Synthetic comm. 12(3) 231-48 (1982):

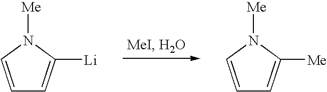

The 2-lithium derivative of N-alkyl pyrroles, such as N-methylpyrrole, was reacted with alkyl or aryl Iodide in Hexane or THF, followed by hydrolysis.

### Preparation of N-Alkyl pyrrole-2-carboxylic Acid Derivatives—General Procedure

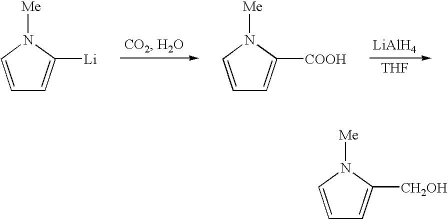

CO₂ powder was added to the 2-lithium derivative of different N-alkyl pyrroles (such as Me, Butyl, hexyl, octyl) at −40° C., to −30° C., followed by addition of water [Jorgenson, org reaction 18 1 (1970)]. The reduction product of the 2-(N-alkyl pyrrole) carboxylic acid was reduced to the corresponding alcohol by LiAlH₄ in THF. The product was identified by ¹H-NMR (data not shown).

The alcohol was attached via esterification to poly acrylic or poly lactic acid to form a pyrrole modified monomer.

The 2-(N-alkyl pyrrole) carboxylic acid was reacted with various PEG molecules to form the corresponding PEG-dipyrrole.

### Preparation of N-(3-hydroxy propyl)pyrrole derivatives—general procedure

N-(2-carboxyethyl)pyrrole, prepared as described above, was reduced by LiAlH₄ in dry THF in a 80% yield, using known procedures. The product was purified by distillation and identified by ¹H-NMR, and EI-MS (data not shown).

The hydroxy pyrrole derivative was attached via esterification to poly acrylic and poly lactic acid to form a pyrrole modified monomer.

Preparation of Pyrrole Conjugates of Modified Carboxylic Acids Containing Saccharide or Polysaccharide—General Procedure:

To allow the conjugation of carboxylic acids modified by saccharide-containing, or polysaccharide-containing agents, to the amino pyrrole, the saccharide is first oxidized to form aldehyde bonds which are then reacted with the aminopropyl pyrrole to form polymerizable pyrrole saccharide derivatives.

Preparation of Pyrrole Conjugates of Modified Carboxylic Acids Containing Hydroxy Groups—General Procedure:

To allow the conjugation of carboxylic acids modified by hydroxy containing active agents, to the amino pyrrole, the amino pyrrole is first esterified using the common activating agents, such as carbodiimides.

Alternatively, the hydroxyl group on the active agents is first conjugated to an amino acid or a short peptide via an ester bond, resulting in an amino or imine derivative thereof, which is then conjugated to the pyrrole either through an amidation reaction, using carbodiimide as a coupling agent, or through an imine bond when using an aldehyde containing pyrrole.

In a typical reaction, amino terminated PEG2000 was reacted with 1.3 equivalents of carboxyethylpyrrole in DMF using DCC as a coupling agent at room temperature for 3 days. The product was isolated by evaporating the DMF to dryness and triturating the residue in diethyl ether. The conjugation yield was over 90% as determined by mass-spectrometry and ¹H-NMR analysis

Preparation of Pyrrole Conjugates of Long Aliphatic Carboxylic Acids—General Procedure:

ω-carboxyalkylyrrole derivatives with longer aliphatic chains are synthesized according to Schuhmann (in Diagnostic Biosensor Polymers, A M Usmani and N. Akmal, eds. ACS Symposium Series 1994, 226, 110, Electroanalysis, 1998, 10, 546-552).

Electropolymerization of pyrrole derivatives on stainless steel surfaces having fatty acid SAM attached thereon was performed as described above. While primary amino containing pyrrole monomers do not surface polymerize, amino-protected derivatives thereof were easily surface polymerized. Protection of the amino group can be performed by any of the common protecting groups used in, for example, peptide syntheses or by forming a Schiff base with acetaldehyde.

Such amino-protected pyrrole derivatives were easily electropolymerized onto dodecanoic acid-treated stainless steel surfaces, to thereby form a strongly adherent coating having free amino groups thereof, which can serve for further conjugation of desired groups, polymers or particles.

### Example 4

### Stainless Steel Surfaces Having Fatty Acid Self-Assembled Monolayers Substituted by a Functional Group Applied Thereon

Fatty acids substituted by a functional group, preferably at the distal end thereof (relative to the carboxylic end) were attached to stainless steel surfaces, so as to form SAMs, as described hereinabove. The modification was carried out in a 0.1M TBATFB/acetonitrile solution containing the functional fatty acid and up to 20% v/v water. Higher water concentrations resulted in less efficient attachment.

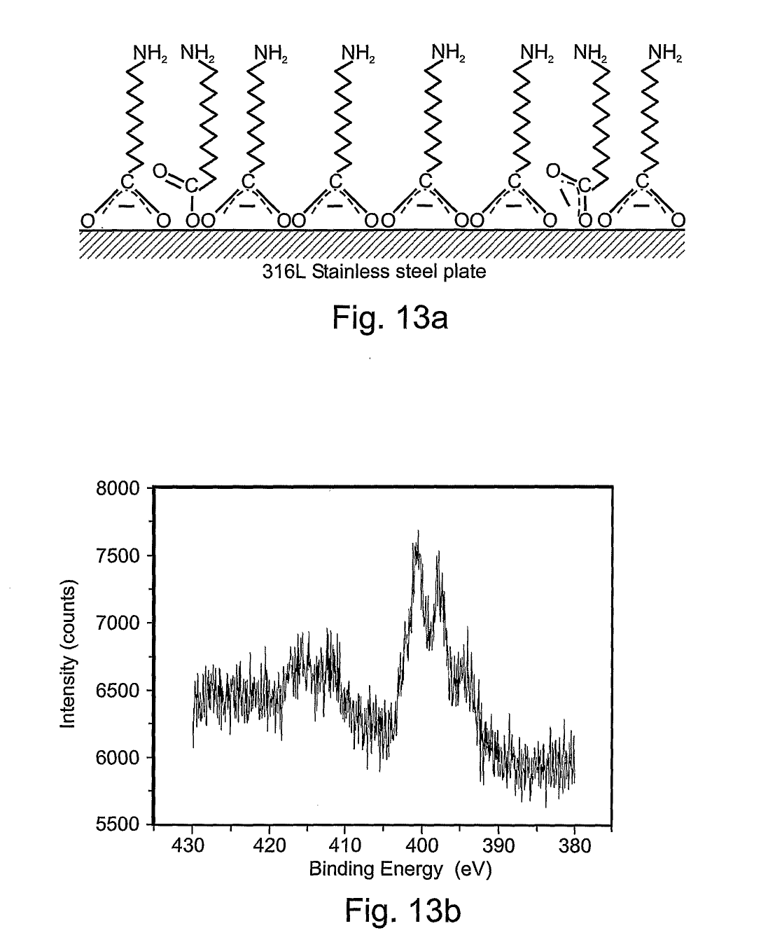

The electrochemical and chemical features of modified stainless steel surfaces were evaluated using RA-FTIR, XPS and contact angle analyses, which indicated that the functional fatty acids are attached to the surface via their carboxylic end, thus living the functional amino group available for further interactions, as is illustrated in FIG. 13a.

Thus, the FTIR spectra of a 12-aminododecanoic acid-treated plate showed two weak absorption peaks at 3500 to 3300 cm⁻¹, corresponding to N—H stretching vibrations, and indicating typical primary amino groups (not shown).

The XPS spectrum of a 12-amino dodecanoic acid-treated plate is presented in FIG. 13b. As can be seen in FIG. 13b, the main peaks at 397.7 400.7 indicate the presence of free amines and charged ammonium groups, respectively, indicating the presence of the amino groups of the plate surface.

### Example 5

### Preparation of Surface-Functionalized Nanoparticles

Surface-functionalized particles, namely, microparticles and nanoparticles having functional groups on their surface, which enable their interaction with a functionalized or non-functionalized conductive surface, can be prepared using two main strategies: (i) forming the particles and thereafter modify the particle surface by conjugating or absorbing thereto molecules or additional polymers having the desired functional groups; and (ii) forming the particles from polymers having the functional groups.

According to the first strategy, surface functionalization of pre-prepared polyester- or polyamide-based particles, particularly those that are based on alkyl hydroxy acids such as lactide and glycolide, is typically performed either by chemical modification of the polymer chains on the surface or by absorbing molecules having functional groups onto the polymer surface. Chemical modification of the polymer chains can be performed, for example, by reacting carboxylic acid and hydroxyl end chain groups with polyethylene glycol (PEG) having diamine or dicarboxylic acid end groups, in the presence of an amide or ester coupling agent such as dicyclohexyl carbodiimide (DCC) or its derivatives. Alternatively, enrichment of the particles surface with active carboxylic acid and hydroxyl groups, can be performed by incubating the particles in an aqueous solution, to thereby induce surface hydrolysis which generates the functional groups. Enrichment of the particles surface with amino functional groups, can be performed using a polyamine such as poly(ethylene imine).

Alternatively, the particles can be dispersed in a solution of amphiphilic molecules containing the functional groups, such that these molecules absorb onto the surface of the particle to thereby provide the desired fictionalizations. In a representative example, a block copolymer of poly(lactide)-poly(ethylene glycol) [PLA-PEG] having amino end groups at the PEG end chain is applied onto the particle surface by dispersing the particle in the PLA-PEG solution, to thereby deposit the polymer onto the particles surface.

While the first strategy detailed above enables a myriad of surface modifications, the second strategy, in which particles with functional groups onto the surface are prepared from polymers pre-bearing the functional groups or functionalized during the particles preparation, is typically preferred, as it enables controlled functionalization and further as it allows the entrapment of bioactive agents into the particles while preparing the desired surface functional particle without the need to expose the drug loaded particle to chemical modification and organic solvents. Such an exposure is highly disadvantageous since it may alter the drug or change the distribution of the drug or its leach-out within or out of the particle.

Various methods have been described in the literature for the formulation of nano- and microparticles having hydrophilic surface such as PEG chain or polysaccharide chains on the surface (see, for example, R. Gref, et al., Poly(ethylene glycol) coated nanospheres, Advanced Drug Delivery Reviews, 16: 215-233, 1995).

In a preferred method, hydrophilic-hydrophobic molecules having functional groups as part of the hydrophilic side are prepared, such that when the molecule is used for the preparation of particles in a mixture of organic-aqueous solvents, the hydrophilic side chain will remain on the surface towards the aqueous medium. For example, PLA-PEG block copolymer having amino groups on the PEG end chain, can be formulated into particles by a solvent evaporation method using PLA and optionally drug solution in an organic solvent dispersed in aqueous solution, to thereby form particles with PEG chains onto the particle surface that have amino functional groups available for further reactions or interactions.

In a representative example, PLA-PEG-amine copolymer (PLA chain MW of about 3,000 D and PEG chain MW of about 1,000 D) was added to a dichloromethane solution of PLAs of various molecular weights, ranging from 3,000 to 50,000 D (10% w/v), at a ratio of 1:10 per PLA in the solution. The resulting clear solution was added drop-wise to a 0.1M phosphate buffer solution pH 7.4 with high-speed homogenization to form a milky dispersion. The mixing was continued for a few hours at room temperature until all solvent was evaporated. The resulted dispersion contained spherical particles of a particle size in a micron range with PEG chains on the surface, as was determined by the ¹H-NMR spectrum of particles dispersed in deuterated water (data not shown). The presence of surface amino groups was determined by reaction of the particles with FITC, a reagents that renders the particles fluorescent. Using the above procedure, drugs such as paclitaxel can be incorporated in the particles by adding the drug to the PLA solution prior to its addition to the aqueous medium for particle preparation. The amount of drug incorporated in the particles can be from about 1% w/w to about 50% of the polymer weight.

Based on the procedure described above, various PLA-PEG diblock copolymers having functional groups on the PEG end chain can be prepared. In one example, PEG diamine (available with a range of molecular weights), protected in one side by a protecting group that can be removed by mild hydrogenation, is reacted with PLA is an organic solution, such that the free PEG amino group and the ester bonds along the PLA chain undergo transamidation to thereby form a PEG-PLA block copolymer and a PLA chain having carboxylic acid end group. The PLA chain length is determined by the ratio between the PEG amino groups and the PLA chain length. The protected amino group is then removed by hydrogenation under mild conditions, which minimally affect the PLA-PEG structure and molecular weight. Alternatively, unprotected PEG diamine is reacted with PLA in solution for a certain time period where mostly one amine end chain is reacted, leaving the other side available for further reactions.

Alternatively, PEG-PLA having various surface functional groups can be prepared by using PEG having one hydroxyl end group and a protected functional group at the other side. The free hydroxyl group is used for the initiation of ring opening polymerization of lactide, glycolide and other reactive lactone monomers. The chain length can be controlled by the ratio between the lactones and the hydroxyls on the PEG chains. The protecting groups are thereafter removed, preferably prior to particle preparation.

Similarly, functional groups such as pyrrole, biotin, carboxylic acids, amino groups and more, as is detailed hereinabove, are added to a hydrophilic polymer chain conjugated to a hydrophobic polymer. For example, polyethylene glycols (PEGs) of molecular weights ranging between 200 and 5000 are conjugated at one end with one or more pyrrole groups and at the other end to a hydrophobic polymer such as poly(lactic acid) (PLA). Dispersing these in aqueous solution, in the presence or absence of other polymers or additives, results in spherical particles of a desired particle size.

In a representative example, polyglycidol-poly(L,L-lactide) block copolymer was prepared according to the procedure described in J. Soc. Perkin Trans 1; EN 12, 1999, 1657-1664.). A protected glycidol monomer (6.95 grams, 0.047 mole) was polymerized by anionic polymerization catalyzed by potassium t-butoxide (0.186 grams, 1.66·10⁻³ mol), in tetrahydrofuran (100 ml), followed by ring opening polymerization of lactide (6.7 grams, 0.046 mol) at reflux conditions, to thereby produce the diblock copolymer. Typical molecular weights for the resulting copolymer range between 6,000 and 7,000 Da with PLA and glycidol blocks of about Mn=3,000.

The protecting groups were thereafter removed by dissolving the copolymer in dioxane-water mixture (ca 150 ml), adding 20 ml of concentrated formic acid and stirring the resulting mixture for 4 days. The obtained copolymer was then frozen and lyophilized, and subjected to functionalization by pyrrole groups as follows:

The free hydroxyl groups along the polymer chain (8 grams) were conjugated with N-pyrrole-propanoic acid (5 grams), using 1-(3-dimethylaminopropyl)-3-ethylcarbodiimide hydrochloride (7.5 grams, Aldrich) and 4-dimethylaminopyridine (DMAP) (0.5 grams, Aldrich) as coupling agents. The reaction was performed in 100 ml dichloromethane, over night.

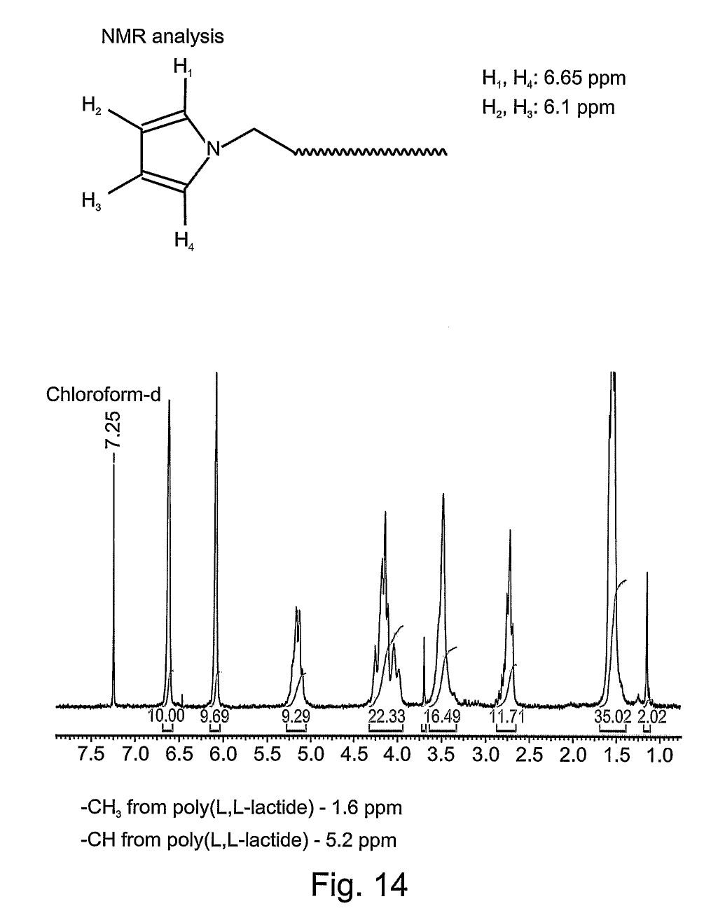

The solvent was thereafter evaporated from the reaction mixture, and the resulting residue was dried, dissolved in 1,4-dioxane and put into Serva SpectraPor dialysis tube. The dialysis purification process was performed during 96 hours and the mixture was thereafter lyophilized. The ¹H-NMR of the obtained pyrrole-functionalized copolymer is presented in FIG. 14 and shows that 75% of the copolymer hydroxyl groups were functionalized by pyrrole groups, indicating the following final product structure (the distribution of both functionalized and non-functionalized glycidol units is statistical):

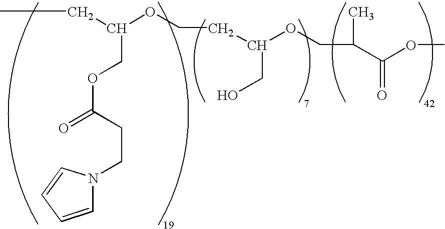

The functionalized nanoparticles were formed as described above, by mixing the functionalized block copolymer with, for example, PEG-PLA and/or poly(lactide) in a chloroform solution, which was thereafter added to a stirring buffer solution (0.01M phosphate pH 7.4), to thereby produce particles having electropolymerizable PEG-pyrrole functional groups on their surface.

The hydroxyl groups of the above PLA-glycidol polymer were also used for the conjugation of various functional groups thereto, which further modify the particles surface. Such a conjugation was performed either before sphere preparation or after sphere preparation. Representative examples of these groups include biotin, which was conjugated by an ester bond and resulted in particles surfaces to which any bioactive agent can be attached via an avidin linker, or which can bind to any avidin-functionalized surface, as is detailed hereinbelow, and dicarboxylic acids, which were conjugated by an ester bond, and resulted in particles surface having carboxylic acid groups thereon, which can be used either for direct electroattachment to metallic surfaces, or for attachment to modified surfaces having hydroxyl or amino groups (e.g., surfaces having 12-aminododecanoic acid SAM applied thereon).

The preparation of biotinilated nanoparticles is highly advantageous as it enables the formation of biotin-avidin layers onto the surface. In a representative experiment, surface biotinilated PEG-PLA particles were prepared as follows:

Biotin was first attached to one side of PEG2000 diamine by dissolving 1 mmol of PEG2000 diamine and 0.1 mmol of Biotin in DMF, adding HOBT and stirring for a few minutes, adding EDC and stirring for 6-10 hours, evaporating the solvent and re-dissolving the residue in ethyl acetate, washing the organic layer with sodium bisulfate 1M and saturated bicarbonate solution, drying over magnesium sulfate and evaporating the solvent.

The thus obtained mono-PEG-Biotin (1 gram) was add to a solution of lactide (1 gram) and Sn-octoate (50 mg) in toluene (10 ml) and the mixture was reacted at 120° C. for 3 hours. The solvent was thereafter evaporated to dryness and the residue was farther reacted at 130° C. to form biotin-PEG-PLA diblock copolymer.

Biotinilated nanoparticles were prepared by dissolving 0.2 grams biotin-PEG-PLA diblock copolymer and 0.4 grams PLA (Mn=3,000) in 5 ml chloroform and adding this solution drop-wise into a 50 ml de-ionized water with overhead stirring at 600 rpm. After solvent evaporation a uniform dispersion of particles was obtained. These particles were electroattached to conductive surfaces as is detailed hereinbelow.

Modification of particles based on non-degradable polymers having functional groups such as esters, amides, amines and imines, e.g., poly(lactide), poly(caprolactone), poly(lactide-glycolide) and the like can also be performed. In a representative example, PLA based nanoparticles, prepared by solvent evaporation as described hereinabove, are placed in water for time period that ranges between a few hours and a few days, so as to hydrolyze the surface functional groups and generate carboxylic acid groups.

Particles having carboxylic acids as their surface groups can be attached to a conductive surface either by direct electroattachment of the carboxylic electroattachable group to the surface, per se or along with fatty acids, or by attachment via a spacer.

The carboxylic acid surface groups can be further used to attach polyamines (e.g., polylysine) thereto.

In a different approach, particles based on stereocomplexes of enantiomeric polymers were prepared, by mixing the solutions of the enantiomeric polymers, e.g., L-PLA and D-PLA, and evaporating the solvent in an aqueous solution. The stereocomplexed particles can be used, for example, for entrapment of bioactive agents therein. In a representative example, two separate solutions one of 10 mg D-PLA (average molecular weight of 60000 Da) dissolved in 10 ml acetonitrile and the other is of 10 mg L-PLA (average molecular weight of 60000 Da) dissolved in 10 ml acetonitrile were mixed at 60° C. for three days. The solution was then evaporated to dryness, yielding white powder of stereocomplexes as nanoparticles.

### Example 6

### Preparation of Surface-Functionalized Nanoparticles Loaded with a Bioactive Agent

Nanoparticles loaded with various bioactive agents can be prepared using any of the procedures described above, while adding the bioactive agent to a polymeric solution (e.g., a PLA solution) prior to its addition to the aqueous medium for particles preparation. The amount of the bioactive agent incorporated in the particles can be from about 1 weight percentage to about 50 weight percentages of the polymer weight.

In a representative example, nanoparticles were prepared by dispersing a chloroform solution of a pyrrole polymer prepared as described hereinabove (0.1 gram), polylactic acid (0.4 grams, Mn=3,000) and paclitaxel (0.15 gram) in de-ionized water at room temperature with constant stirring. After solvent evaporation, nanoparticles of about 200 nanometers were obtained in more than 90% yield. The PEG-pyrrole side chains onto the particles were detected by ¹H NMR conducted in deuterated water (data not shown).

Observing the release rate of paclitaxel from the particles described above, when immersed in 0.1N phosphate buffer solution, indicated that the drug was released constantly from these particles during two weeks.

In another example, paclitaxel was loaded into nanoparticles of stereocomplexes by adding it to the mixture of solutions while mixing as described above. Typically, 6 mg of paclitaxel (10 weight percentages in 1 ml of acetonitrile) were added and mixture was performed as described above until a white solution was obtained. The loaded stereocomplexes nanoparticles were separated by centrifugation, yielding a white powder.

### Example 7

### Conductive Surfaces Having Nanoparticles Applied Thereon

Conjugation of Pyrrole-Substituted Nanoparticles to Stainless Steel Surfaces Having Fatty Acid Self-Assembled Monolayers Applied thereon:

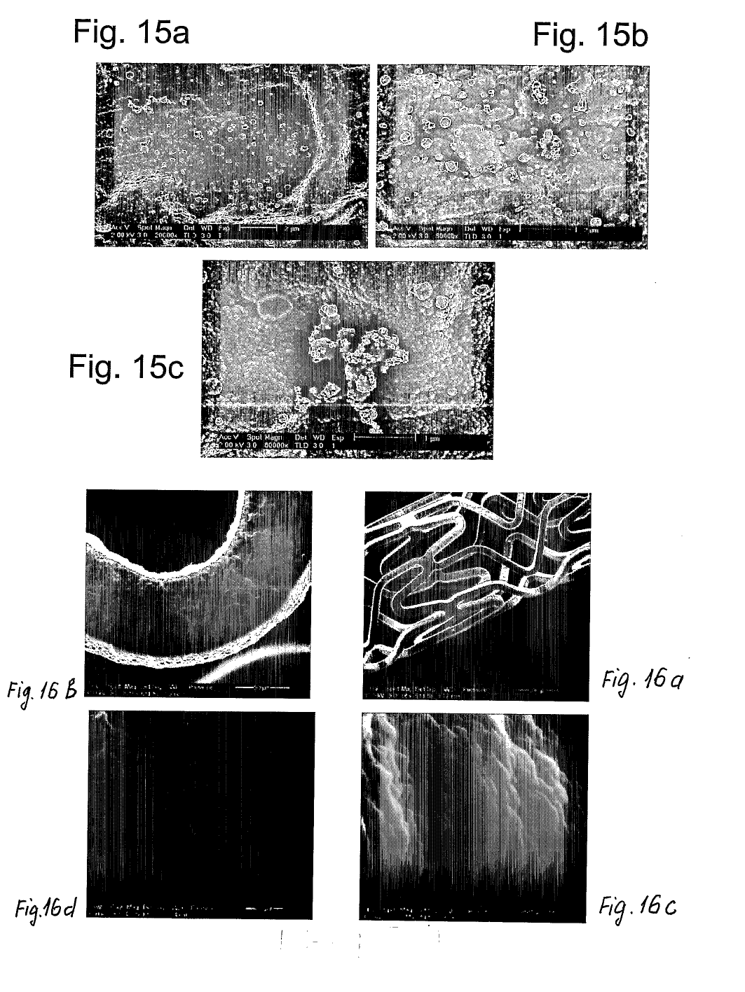

The pyrrole-substituted nanoparticles described above were attached to stainless steel plates pre-treated with decanoic acid, prepared as described hereinabove, by electropolymerization of the pyrrole groups, according to the procedure described above. The morphology of the obtained coated plates was compared with that of stainless steel plates treated with decanoic acid and pyrrole per se, using SEM measurements. FIGS. 15a-c present SEM micrographs of pyrrole-substituted nanoparticles-coated stents, which demonstrate the efficient conjugation of the nanoparticles to the metal surface, as well as the improved surface density and smoothness obtained thereby, particularly when compared to unsubstituted pyrrole-coated plates (see, for example, FIGS. 12a-c).

Using the same process, a stainless steel 316 LM stent (12×1 mm, by STI, Israel) was electrochemically treated with decanoic acid, to thereby form a SAM adherent layer thereon, and was thereafter further coated by electropolymerization of the pyrrole-substituted nanoparticles described hereinabove. FIGS. 16a-c present SEM micrographs of the thus electrocoated stent, demonstrating the smooth and effective coating obtained by this process.

Conjugation of Nanoparticles to Modified Conductive Surfaces:

Nanoparticles having functional groups attached to their surface, as described hereinabove, can be attached to conductive surfaces (e.g., metal surfaces) that are functionalized by complementary groups.

In a representative example, metal plates modified so as to have carboxylic acids functional groups were reacted with particles having surface amino groups, using DCC as activating agent, to thereby attach the particles to the surface via a covalent, yet biodegradable, amide bond. Similarly, metal plates modified so as to have carboxylic acids functional groups were reacted with polymer chains such as, for example, amino terminated poly(lactic acid), using DCC, N-succinamide or other activating agents commonly used in peptide syntheses. Using the same process, other polymers having accessible amino groups, such as for example polysaccharides, which are optionally attached to bioactive agents, can be conjugated to such modified metal surfaces.

Alternatively, PLA nanoparticles, which have free carboxylic group on their surface were directly attached to 316L stainless steel plates, using the procedure described in the methods section above.

In another representative example, metal plates modified so as to have amino functional groups (e.g., having 12-aminododecanoic acid SAM applied thereof, as is described in Example 3) were reacted with particles having surface carboxylic acid groups, using DCC as activating agent, to thereby attach the particles to the surface via a covalent, yet biodegradable, amide bond. The amino groups were also used for the conjugation of poly(lactic acid) chains or nanoparticles having carboxylic acid groups on the surface via amide bonds using coupling agents such as DCC or activated carboxylic acid groups with N-succinamide or other activating agents commonly used in peptide synthesis.

In another representative example, metal plates modified so as to have amino functional groups (e.g., having 12-aminododecanoic acid SAM applied thereof, as is described in Example 3) were reacted with particles having surface carboxylic acid groups, under acidic conditions, to thereby attach the particles to the surface via electrostatic bonds.

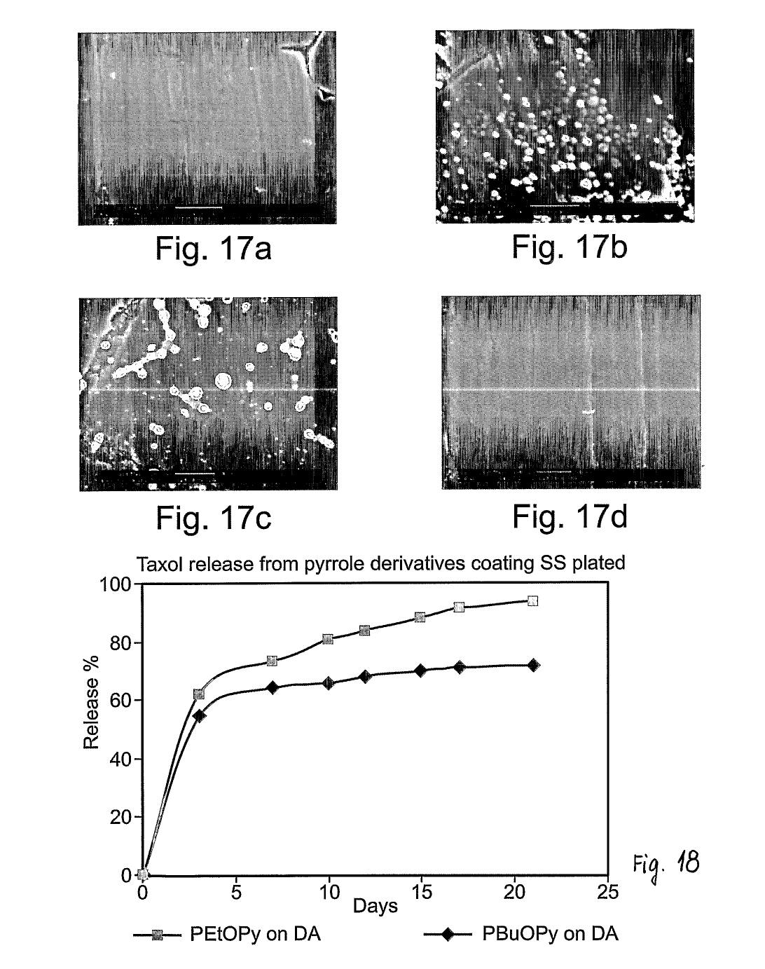

Thus, stainless steel plates pre-treated with 12-aminododecanoic acid were incubated with a dispersion of PLA nanoparticles in a buffer solution overnight. SEM micrographs of the surface of the resulting plates are presented in FIG. 17 and demonstrate the attachment of the particles to the surface.

Alternatively, metal plates modified so as to have carboxylic acids functional groups are further modified by converting the carboxylic acid functional groups into amino groups, which are thereafter reacted with, for example, bioactive agents having a carboxylic group, polymer chains having free carboxylic groups or nanoparticles enriched with carboxylic acid groups. The nanoparticles can be loaded with a bioactive agent as well. Modification of the metal plate carboxylic groups to amino groups is performed, for example, by reacting activated carboxylic acids with a polyamine such as poly(alkanediamine), PEG-diamines or polyethylene imine, which forms a layer of multi amino groups on the surface.

Further alternatively, metal plates modified so as to have hydroxyl or amino functional groups are used as an initiator for ring opening polymerization of lactones, such that by reacting such metal plates with, for example, lactide and/or glycolide, a polymeric coating is applied onto the metal surface. In an exemplary procedure, a hydroxyl or amino functionalized metal surface of e.g., a stent, is immersed in a solution of lactide in toluene. After partial evaporation of the toluene, a catalytic amount of staneous octoate is added so as to initiate polymerization induced by the hydroxyl or amine groups, to thereby provide a PLA-coated metal surface. Such a polymeric coating can be used for attachment of a bioactive agent to the polymer, by, for example, swelling the coated object with a concentrated solution of the bioactive agent (e.g., paclitaxel) and thereafter evaporating the solvent. This process can be further used to obtain, for example, a polysaccharide-coated metal surface.

Further alternatively, polymeric coating of metal surfaces can be performed by modifying a metal surface so as to have acrylate or other vinyl functional groups, and thereafter react the metal surface with particles or polymers having accessible vinyl groups, to thereby provide a metal surface coated by the polymer or particle. Such vinyl polymerizations can be used to improve the mechanical strength of the coating.

Using another approach, nanoparticles were attached to conductive surfaces by grafting into an electropolymerized polymer. In a representative example, pyrrole was electropolymerized onto a stainless steel surface in the presence of poly(lactide co glycidol) particles and OxA, as is described in the methods section hereinabove. OxA was added to the modification solution since it enhances the adherence of pyrrole to the surface.

Each of the processes described above is preferably performed using metal surfaces having a functionalized fatty acid SAM applied thereon.

### Example 8

### Conductive Surfaces Having Bioactive Agents Attached Thereto

As is described herein throughout, bioactive agents can be incorporated into the coated surfaces described herein using a variety of reactions and interactions, such as, covalent bonding, electrostatic bonding, encapsulation, absorption or swelling, between the bioactive agent and a functionalized surface, particles attached to a functionalized or non-functionalized surface, electropolymerizable polymers attached to a functionalized surface, and so on.

Following are representative examples in which bioactive agents were attached to stainless steel surfaces:

Preparation of Plates and Stents Having Substituted Polypyrrole Applied Thereon and Paclitaxel Absorbed within the Polypyrrole:

Substituted polypyrrole was used for electrocoating stainless steel plates and stents pre-treated with decanoic acid, as described hereinabove. Absorption of paclitaxel into the coating was performed by diffusion from a methanolic solution. The drug was held by hydrophobic-hydrophobic interaction between the drug and the polymer coating. The coating remained intact and uniform without any notice of surface change or swelling during the absorption process.

Thus, paclitaxel (Taxol) was dissolved in methanol (15 mg/ml) and the polymer coated plates or stents were immersed in drug solution for 2 hours. The plate/stent was thereafter removed from the solution and air-dried. The plate/stent was dipped additional two times in the same solution for 5 minutes each time and dried.

The drug loading in the devices coated by the various techniques was determined by extracting out the drug, using an ultrasonic bath containing methanol and placing the loaded plate/stent therein.

The release characteristic of the stent or plate was determined by placing drug-loaded device into buffer phosphate solution, pH 7.4, which contained 0.3% SDS, to increase the solubility of paclitaxel, and the daily release was determined by HPLC.

Alternatively, paclitaxel was added to the electropolymerization solution during the electroattachment of pyrrole to the plate, as described hereinabove.

Further alternatively, paclitaxel was added to the electropolymerization solution and the electropolymerization was performed in steps, as follows:

The monomer was subjected to 3 CV cycles and the plate/stent was immersed in a taxol solution (15 mg/ml) for 2 minutes, followed by electropolymerization in the monomer solution for 3 CV cycles and immersing in the drug solution for two minutes, and so on, for about 4 times.

Further alternatively, polypyrrole-coated plate/stent were immersed in a taxol solution further containing PLA particles, and thereafter the drug-loaded device was immersed in PLA solution, for additional coating.

Further alternatively, nanoparticles entrapping paclitaxel in stereocomplexes of D-PLA and L-PLA, prepared as described above were added to the electropolymerization solution.

The obtained results indicated that a maximum of drug loaded into the polymer was indicated. For drug absorbed by immersing the plates or stents into the drug solution, about 143 μg for poly(ethyl ester)pyrrole on decanoic acid precoating (DA) and about 100 μg for poly(butyl ester)pyrrole on DA of loaded drug were observed. For drug absorbed during electropolymerization where paclitaxel is in the electropolymerization solution about 25 μg of drug was loaded for poly(butyl ester)pyrrole on DA (release of the drug after this experiment not shown).

The results obtained for the release of taxol from stents/plates coated with polypyrrole derivatives is shown in FIG. 18. After 3 days about 50% of the absorbed drug has been released into the buffer medium, whereby the remaining drug released slowly for over 3 weeks.

It should be noted that the pre-coating of decanoic acid, not only improved the adhesion and stability of the coating but also increased the amount of paclitaxel on the stent by at least 50%. Also, the release rate from the pre-coated decanoic acid was much more controlled compared to the coated pyrrole derivatives without the pre-coating.

Preparation of Plates and Stents Having Biotin-Avidin Complexes Attached Thereto:

Stainless steel surfaces coated with multi-layered biotin-avidin complexes were prepared as follows:

As a first step biotin or avidin were attached to the surface, either directly, via a carboxylic acid moiety (for biotin) or indirectly, by electropolymerization of pyrrole substituted by biotin or avidin onto fatty acid pre-treated plate, or by conjugation to functionalized fatty acid SAM (e.g., formation of amide bond between 12-aminododecanoic acid).

Thereafter, avidin was attached to the biotinilated surface, and biotinilated particles or polymers, prepared as described above were attached to the avidin-activated sites.

Addition of avidin to the biotinilated surface can result in additional avidin layer, which may further be conjugated to biotinilated particles or polymers and so on.

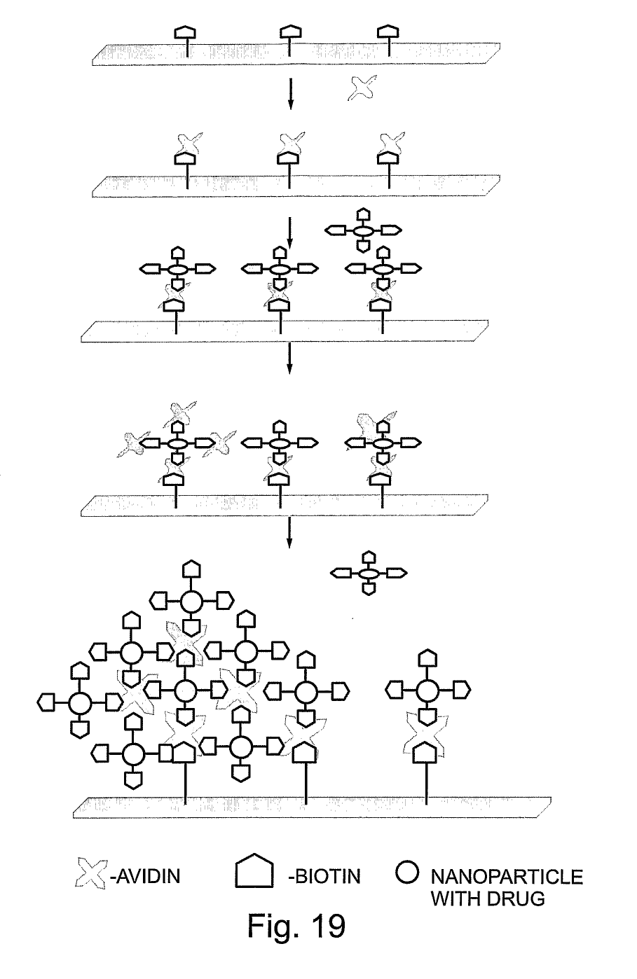

The above process is schematically illustrated in FIG. 19 and clearly demonstrated the capability of the biotin-avidin complex to encompass a large number of the active substance molecules, and thus obtain an area characterized by an unusually high drug concentration.

In a typical experiment, stainless steel 316 LM plates were coated with biotin carboxylic acid by electrocoating of the biotin with or without decanoic acid. The plate was incubated with a 1 mg/ml solution of avidin overnight. The avidin presence onto the plate was recognized by complexing with biotin-FITC fluorescent dye. The avidin coated plate was placed in the dispersion of biotin-PEG-PLA nanoparticles for 5 hour at room temperature. SEM analysis indicated the presence of nanoparticles on the surface (data not shown).

Using the above technique, bioactive agents attached to biotin or avidin can be incorporated into the coating at any stage.

### Example 9

### Conductive Surfaces Having Multi-Layered Coatings Attached Thereto

In this example, the preparation of electropolymerizable monomers and electropolymerized formed therefrom, which can be deposited on the surface via SAMs, and be utilized for forming multi-layered coating in which active substances can be efficiently and controllably loaded is described.

To that end, three general approaches were designed and practiced, as follows:

(i) bifunctional monomers, having an electropolymerizable moiety and a chemically polymerizable group, were prepared and subjected to a two-step polymerization process via the SAMs: electrochemical polymerization via the SAMs, followed by a chemical polymerization (e.g., free radical polymerization in the presence of a catalyst);

(ii) electropolymerizable bifunctional monomers having a photoreactive group (PAG) were prepared and subjected to a two-step polymerization process via the SAMs: electrochemical polymerization via the SAMs, which resulted in activated polymer, followed by a chemical polymerization, which is catalyzed by irradiation and induced by the activated polymer, and is performed in the presence of another monomer and/or a drug; and

(iii) electropolymerizable bifunctional monomers having a reactive group were prepared and subjected to a two-step polymerization process via the SAMs: electrochemical polymerization via the SAMs, which resulted in activated polymer, followed by a chemical polymerization, in the presence of a catalyst, and another monomer and/or a drug, in which the reactive group participates.

In addition to the above, multi-layered coatings were also obtained by a simple multi-step polymerization process via the SAMs, which included one or more consecutive electrochemical polymerization processes, optionally followed by impregnation of an additional non-electropolymerizable polymer, as described hereinabove.

In each of the above procedures, the final multi-layered stent can be immersed in a drug solution for drug loading. Alternatively, the drug can be loaded during one or more of the chemical polymerization processes by adding the drug to the polymerization solution.

Two-Step Polymerization Route Via Chemically Active Groups of Pyrrole Derivatives:

Vinyl derivatives of pyrrole were prepared by reacting N-(2-carboxyethyl) pyrrole with allyl alcohol to yield the corresponding allyl ester in 60% yield, or by reacting N-(2-carboxyethyl) pyrrole with acryloyl chloride in dichloromethane and in the presence of triethylamine [as described in Min Shi et al., molecules 7 (2002)]. The vinyl pyrrole derivative was electrochemically polymerized on the SAM, via the 2 and 5-positions of the pyrrole unit, resulting in a polymer having free vinyl groups attached thereto. This polymer was further polymerized in the presence of AIBN or benzoyl peroxide as initiators for free radical polymerization of the monomer, as follows:

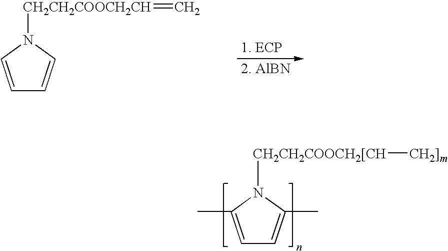

This general approach was described, for example, for the free radical polymerization of N-vinyl pyrrole with AIBN, followed by second polymerization with FeCl₃ [see, for example, Ruggeri et al Pure and appl chem. 69 (1) 143-149 (1997)].

Preparation of Electropolymerized Polymers Having Photoreactive Groups (PAG) Attached Thereto:

An electropolymerizable pyrrole monomer having a benzophenone derivative, as an exemplary photoreactive group, was prepared by an esterification reaction between N-(2-carboxyethyl) pyrrole and a benzophenone reactive derivative, such as 2-hydroxy-4-methoxy-benzophenone, in toluene, using para-toluene sulfonic acid as a catalyst, and Na₂SO₄ and MgSO₄ as desiccants.

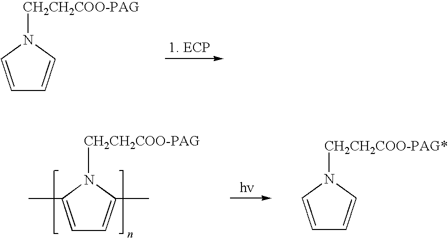

Following electrochemical polymerization via the SAMs, polypyrrole having benzophenone groups attached thereto was obtained. This polymer was activated by irradiation, to allow an additional, chemical polymerization process, which is induced by the activated groups.

Polyacrylate-Containing Multi-Layered Coatings:

Double-layered drug-loaded polyacrylate-containing coatings on stents were prepared is order to improve the mechanical properties of the polypyrrole coating and/or to improve the total loading and to optimize the releasing profile from the stents coated by polypyrrole derivatives.

Such double-layered coated stents were prepared using two methods as follows:

Method 1: polypyrrole-coated stents were obtained as described above, using a mixture of 1:7:2 (molequivalents) PPA, PPA butyl ester and PPA hexyl ester as the electropolymerization solution and were thereafter immersed in solution of 40 mg/ml paclitaxel and 1% polymethyllauryl (2:3) methacrylate in chloroform for one minute. Then, the stents were dried and immersed again for one minute in the same solution, and were finally dried again. Thereafter, the dry stents were immersed in a solution of 1% polymethyllauryl (2:3) methacrylate in cyclohexane for 20 seconds.

Total drug loading was 85-100 μg on each stent.

The coating thickness was about 0.8 μm.

Method 2: polypyrrole-coated stents were obtained as described above, using a mixture of 1:7:2 (molequivalents) PPA, PPA butyl ester and PPA hexyl ester as the electropolymerization solution and were thereafter immersed in a solution containing 30 mg/ml paclitaxel in ethanol for 30 minutes. Stents were thereafter immersed in a solution containing 40 mg/ml paclitaxel and 1% polymethyllauryl (2:3) methacrylate in chloroform for one minute, and dried. The dry stents were then immersed in a solution containing 1% polymethyllauryl (2:3) methacrylate in cyclohexane for 20 seconds.

Total drug loading was 85-110 μg on each stent.

The coating thickness was about 0.8 μm.

Poly(allyl ester) Pyrrole Coating Modification with Lauryl Methacrylate and PETMA, on Stents:

Bifunctional monomers such as the allyl ester derivative of pyrrole described hereinabove, which contains pyrrole units were used to obtain stents coated with poly(allyl ester)pyrrole. The coating thickness was 0.4 μm. Modification of the stent surface by another polymerization of an acrylate monomer was then performed as follows:

Polymerization of Lauryl Methacrylate (Benzoyl peroxide (BP) as initiator): To a lauryl methacrylate (LM) monomer solution (either neat or 50% LM in DCM), 1% w/v of BP per monomer was added. The allyl ester polypyrrole-coated stent was immersed in the solution for 5 seconds. Then the stent was dried to remove excess of the LM solution and inserted to an empty small glass vial under stream of nitrogen for some minutes. The vial was closed and heated to 70° C. for 5 hours. After the reaction was completed the stent was rinsed with methanol and expanded. A uniform coating was obtained.

Crosslinked polymerization of Lauryl Methacrylate with PETMA (pentaeritritoltetrametacrylate) (BP as initiator): Using the same procedure as above, a cross-linked polyacrylate coating was obtained by adding to the acrylate monomer solution 1% w/w PETMA as a cross-linking agent.

Polymerization of PETMA (BP as initiator): Using the same procedure as above, a cross-linked polymer coating was obtained by using a solution of 50% PETMA in DCM as the monomer solution.

Polymerization in aqueous medium: each of the procedures described above was performed by immersing the stent in the monomer solution, drying the stent and immersing the resulting stent in water under nitrogen stream. Then 0.25% of Na₂S₂O₅, 0.25% of FeH₈N₂O₈S₂ and Na₂S₂O₈ were added and the mixture was stirred for 5 hours. The stent was then rinsed with water and expanded.

Each of the electropolymerization processes described hereinabove (e.g., in Examples 3-5), can be performed on stents or on other implantable devices, as well as on certain parts of the device. For example, the inner part of a metal stent can be protected from electropolymerization coating by inserting the stent onto an inflated baloon or a soft or rigid rod, thus limiting the access of the electropolymerization solution to the inner side of the stent. Likewise, the inner part can be electrochemically coated without coating the surface, by covering the outer part with a balloon or a soft cover. A device can be coated by various coating layers to allow the desired properties. For example, the initial polymerization layer can be composed of pyrrole and N-PEG200-pyrrole monomers at a ratio of 9:1, the second layer can be a mixture of pyrrole:N-alkylpaclitaxel-pyrrole at a ratio of 6:4, and the third layer can be a pyrrole:N-PEG2000-pyrrole mixture at a ratio of 9:1. This type of multilayer coating provides a release of paclitaxel over time, which is controlled by the cleavage of the agent from the pyrrole unit in the polymer and diffusion through the outer layer which also serves as passive protection from tissue and body fluids.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED BY NUMERALS

### Other References are Cited within the Text

1. J. A. Helsen, H. J. Breme, editors, Metals as Biomaterials, New York, Wiley, 1998.
2. U. Harm, R. Burgler, W. Furbeth, K.-M. Mangold, K. Juttner, Macromol. Symp., 2002, 187, 65.
3. C. Reinartz, W. Furbeth, M. Stratmann, Fresenius J. Anal. Chem., 1995, 353, 657.
4. M. Stratmann, M. Volmeruebing, Appl. Surf. Sci., 1992, 55, 19.
5. C. M. Ruan, T. Bayer, S. Meth, C. N. Sukenik, thin solid films, 2002, 419, 95.
6. J. Marsh, J. D. Scantlebury, S. B. Lyon, J. Appl. Polym. Sci., 1996, 59, 897.
7. A. Ulman, An Introduction to Ultrathin Organic films from Langmuir-Blodgett to Self-Assembly, Academic Press, New York, 1991.
8. F. Schriber, Prog Surf. Sci., 2000, 65, 151.
9. L. Netzer, J. Sagiv, J. Am. Chem. Soc., 1983, 105, 674.
10. N. Tsuji, K. Nozawa, K. Aramaki, Corros. Sci., 2000, 42, 1523.
11. K. Aramaki, Corros. Sci., 1999, 41, 1715.
12. K. Nozawa, K. Aramaki, Corros. Sci., 1999, 41, 57.
13. K. Nozawa, H. Nishihara, K. Aramaki, Corros. Sci., 1997, 39, 1625.
14. W. Xing, Y. Shan, D. Guo, T. Lu, S. Xi, Corrosion, 1995, 51, 45.
15. D. D. Domiguez, R. L. Mowery, N. H. Turner, Tribol. Trans., 1994, 37, 59.
16. S-.Y. Lin, T. K. Tsai. C. M. Lin, C. H. Chen, Langmuir, 2002, 18, 5473.
17. M. G. Samant, C. A. Brown, J. G. Gordon, Langmuir, 1993, 9, 1082.
18. M. G. Samant, C. A. Brown, J. G. Gordon, Langmuir, 1991, 7, 437.
19. Y. T. Tao, W. L Lin, J. Phys. Chem. B, 1997, 101, 9732.
20. D. L. Allara, R. G. Nuzzo, Langmuir, 1985, 1, 45.
21. D. L. Allara, R. G. Nuzzo, Langmuir, 1985, 1, 52.
22. Y. T. Tao, G. D. Hietpas, D. L. Allara, J. Am. Chem. Soc., 1996, 118, 6724.
23. Y. T. Tao, M. T. Lee, S. C. Chang, J. Am. Chem. Soc., 1993, 115, 9547.
24. D. L. Allara, B. T. Bright, M. D. Porter, N. E. Schlotter, Chem. Phys. Lett., 1986, 132, 93.
25. Y. T. Tao, J. Am. Chem. Soc., 1993, 115, 4350.
26. Gritzner, G.; Kuta, J. Pure. Appl. Chem., 1984, 56, 461.
27. T. R. Jervis, J.-P. Hirvonen, Wear, 1991, 150, 259.
28. T. Ohmi, M. Morita, A. Teramoto, K. Makihara, K. S. Tseng, Appl. Phys. Lett., 1992, 60, 2126.
29. M. Nieminen, L. Niinisto, E. Rauhala, J. Mater. Chem., 1996, 6, 27.
30. L. Niinisto, M. Ritala, M. Leskela, Mater. Sci. Engineer., 1996, 41, 23.
31. H. Brunner, T. Vallant, U. Mayer, H. Hoffmann, Langmuir, 1996, 12, 4614.
32. H. Ron, I. Rubinstein, J. Am. Chem. Soc., 1998, 120, 13444.
33. H. Ron, I. Rubinstein, Langmuir, 1994, 10, 4566.
34.1. Turyan, D. Mandler, unpublished results.
35. A. J. Bard, L. R Faulkner, “Electrochemical Methods, Fundamentals and Applications”, 2^{nd} Edition, 2001, pp. 230-250.
36. H. O. Finklea, in “Electroanalytical Chemistry—a Series of Advances”, (A. J. Bard Ed.) Vol 19, 1996, pp. 110-318.
37. E. Laibinis, G. M. Whitesides, D. A. Allara, Y. T. Tao, A. N. Parikh, R. G. Nuzzo, J. Am. Chem. Soc., 1991, 113, 7152.
38. C. Amatore, J.-M. Saveant, D. Tessier, J. Electroanal. Chem., 1983, 147, 39.

What is claimed is:

1. An article-of-manufacture comprising an object having a conductive surface and at least one active substance being attached to at least a portion of said conductive surface, wherein said conductive surface is a modified conductive surface having at least one functional moiety capable of interacting with said at least one active substance and/or said at least one active substance is electrochemically attached to said conductive surface.

2. The article-of manufacture of claim 1, wherein said object is a medical device.

3. The article-of-manufacture of claim 2, wherein said object is an implantable device.

4. The article-of-manufacture of claim 3, wherein said implantable device is selected from the group consisting of a pacemaker, a graft, a stent, a wire, an orthopedic implant, an implantable diffusion pump, an injection port and a heart valve.

5. The article-of-manufacture of claim 4, wherein said implantable device is a stent.

6. The article-of-manufacture of claim 1, wherein said conductive surface comprises at least one metal or an alloy thereof.

7. The article-of-manufacture of claim 6, wherein said at least one metal is selected from the group consisting of iron, stainless steel, titanium, nickel, tantalum, platinum, gold, silver, copper and any combination thereof.

8. The article-of-manufacture of claim 7, wherein said conductive surface comprises stainless steel.

9. The article-of-manufacture of claim 1, wherein said at least one active substance is selected from the group consisting of a bioactive agent, a polymer, a polymer having a bioactive agent attached thereto, a plurality of microparticles and/or nanoparticles, a plurality of microparticles and/or nanoparticles having a bioactive agent attached thereto, and any combination thereof.

10. The article-of-manufacture of claim 9, wherein said bioactive agent is selected from the group consisting of a therapeutically active agent and a labeling agent.

11. The article-of-manufacture of claim 10, wherein said therapeutically active agent is selected from the group consisting of an anti-thrombogenic agent, an anti-platelet agent, an anti-coagulant, a growth factor, a statin, a toxin, an antimicrobial agent, an analgesic, an anti-metabolic agent, a vasoactive agent, a vasodilator agent, a prostaglandin, a hormone, a thrombin inhibitor, an enzyme, an oligonucleotide, a nucleic acid, an antisense, a protein, an antibody, an antigen, a vitamin, an immunoglobulin, a cytokine, a cardiovascular agent, endothelial cells, an anti-inflammatory agent, an antibiotic, a chemotherapeutic agent, an antioxidant, a phospholipid, an anti-proliferative agent, a corticosteroid, a heparin, a heparinoid, albumin, a gamma globulin, paclitaxel, hyaluronic acid and any combination thereof.

12. The article-of-manufacture of claim 1, wherein said conductive surface is an electrochemically modified conductive surface having at least one functional moiety capable of interacting with said at least one active substance.

13. The article-of-manufacture of claim 1, wherein said conductive surface is a non-electrochemically modified conductive surface having at least one functional moiety capable of interacting with said at least one active substance.

14. The article-of-manufacture of any of claims 12 and 13, wherein said interacting is effected by a covalent bond, a biodegradable bond, a ionic bond, a hydrogen bond, Van der Waals interactions, hydrophobic interactions, swelling and absorption.

15. The article-of-manufacture of any of claims 12 and 13, wherein said at least one functional moiety is selected from the group consisting of amine, ammonium ion, carboxylate, thiocarboxylate, amide, carbamyl, hydroxyl, thiohydroxyl, alkoxide, thioalkoxide, nitrate, cyanate, pyrrole, isocyanate, halide, azide, an unsaturated moiety, a hydrophobic moiety, phosphate, phosphonate, sulfate, sulfonate, sulfonamide, and any combination thereof.

16. The article-of-manufacture of any of claims 1, 12 and 13, wherein said conductive surface is modified by attaching thereto at least one organic substance.

17. The article-of-manufacture of claim 16, wherein said at least one organic substance forms a self-assembled monolayer onto said conductive surface.

18. The article-of-manufacture of any of claims 12-17, wherein said conductive surface is electrochemically modified by electrochemically attaching thereto said at least one organic substance, whereas said organic substance comprises an electroattachable group and a functional moiety capable of interacting with said active substance.

19. The article-of-manufacture of claim 18, wherein said electroattachable group is selected from the group consisting of a carboxylate, a sulfonate, a sulfate, a phosphonate and a phosphate.

20. The article-of-manufacture of claim 18, wherein said at least one organic substance further comprises an organic residue having 3-30 carbon atoms.

21. The article-of-manufacture of claim 20, wherein said at least one organic substance is selected from the group consisting of a fatty acid and a fatty acid derivatized by said functional group.

22. The article-of-manufacture of any of claims 12-17, wherein said conductive surface is non-electrochemically modified by depositing thereon at least one organic substance, whereas said at least one organic substance comprises an organosilane having a functional moiety capable of interacting with said active substance.

23. The article-of-manufacture of claim 22, wherein said organosilane has the general formula:
XmSiR(4-m)
whereas:
m is an integer from 1 to 3;
X is selected from the group consisting of halide, alkoxy and thioalkoxy; and
R is a substituted or unsubstituted, saturated or unsaturated hydrocarbon residue.

24. The article-of-manufacture of claim 23, wherein said hydrocarbon residue has from 1 to 10 carbon atoms.

25. The article-of-manufacture of claim 1, wherein said at least one active substance is electrochemically attached to said conductive surface.

26. The article-of-manufacture of claim 25, wherein said at least one active substance is selected from the group consisting of a bioactive agent, a polymer, a polymer having a bioactive agent attached thereto, a plurality of microparticles and/or nanoparticles, a plurality of microparticles and/or nanoparticles having a bioactive agent attached thereto, and any combination thereof, said bioactive agent, said polymer, said microparticles and/or said nanoparticles comprising at least one electroattachable group.

27. The article-of-manufacture of claim 26, wherein said therapeutically active agent is selected from the group consisting of a therapeutically active agent and a labeling agent.

28. The article-of-manufacture of claim 27, wherein said therapeutically active agent is selected from the group consisting of an anti-thrombogenic agent, an anti-platelet agent, an anti-coagulant, a growth factor, a statin, a toxin, an antimicrobial agent, an analgesic, an anti-metabolic agent, a vasoactive agent, a vasodilator agent, a prostaglandin, a hormone, a thrombin inhibitor, an enzyme, an oligonucleotide, a nucleic acid, an antisense, a protein, an antibody, an antigen, a vitamin, an immunoglobulin, a cytokine, a cardiovascular agent, endothelial cells, an anti-inflammatory agent, an antibiotic, a chemotherapeutic agent, an antioxidant, a phospholipid, an anti-proliferative agent, a corticosteroid, a heparin, a heparinoid, albumin, a gamma globulin, paclitaxel, hyaluronic acid and any combination thereof.

29. The article-of-manufacture of claim 26, wherein said electroattachable group is selected from the group consisting of a carboxylate, a sulfonate, a sulfate, a phosphonate and a phosphate.

30. The article-of-manufacture of claim 1, wherein said conductive surface is a modified conductive surface having at least one functional moiety capable of interacting with said active substance and said at least one active substance is electrochemically attached to said modified conductive surface.

31. The article-of-manufacture of claim 30, wherein said at least one functional moiety is selected from the group consisting of amine, ammonium ion, carboxylate, thiocarboxylate, amide, carbamyl, hydroxyl, thiohydroxyl, alkoxide, thioalkoxide, nitrate, cyanate, isocyanate, pyrrole, halide, azide, an unsaturated moiety, a hydrophobic moiety, phosphate, phosphonate, sulfate, sulfonate, sulfonamide, and any combination thereof.

32. The article-of-manufacture of claim 31, wherein said at least one functional moiety is a hydrophobic moiety

33. The article-of-manufacture claim 30, wherein said conductive surface is modified by attaching thereto at least one organic substance.

34. The article-of-manufacture of claim 33, wherein said at least one organic substance forms a self-assembled monolayer onto said conductive surface.

35. The article-of-manufacture of any of claims 30-34, wherein said conductive surface is modified by electrochemically attaching thereto at least one organic substance, said organic substance having an electroattachable group and a functional moiety capable of interacting with said active substance.

36. The article-of-manufacture of any of claims 30-34, wherein said conductive surface is modified by depositing thereon an organosilane, said organosilane having a functional moiety capable of interacting with said active substance.

37. The article-of-manufacture of claim 36, wherein said organosilane has the general formula:
XmSiR(4-m)
whereas:
m is an integer from 1 to 3;
X is selected from the group consisting of halide, alkoxy and thioalkoxy; and
R is a substituted or unsubstituted, saturated or unsaturated hydrocarbon residue.

38. The article-of-manufacture of claim 37, wherein said hydrocarbon residue has from 1 to 10 carbon atoms.

39. The article-of-manufacture of claim 35, wherein said electroattachable group is selected from the group consisting of a carboxylate, a sulfonate, a sulfate, a phosphonate and a phosphate.

40. The article-of-manufacture of claim 35, wherein said at least one organic substance further comprises an organic residue having 3-30 carbon atoms.

41. The article-of-manufacture of claim 40, wherein said at least one organic substance is selected from the group consisting of a fatty acid and a fatty acid derivatized by said at least one functional group.

42. The article-of-manufacture of claim 30, wherein said active substance is an electropolymerized polymer.

43. The article-of-manufacture of claim 42, wherein said electropolymerized polymer comprises a bioactive agent attached thereto.

44. The article-of-manufacture of claim 42, wherein said electropolymerized polymer comprises a plurality of microparticles and/or nanoparticles attached thereto.

45. The article-of-manufacture of claim 44, wherein said plurality of microparticles and/or nanoparticles comprises a bioactive agent being attached thereto.

46. The article-of-manufacture of claim 42, wherein said electropolymerized polymer comprises a co-polymer attached thereto.

47. The article-of-manufacture of claim 46, wherein said co-polymer comprises a bioactive agent being attached thereto.

48. The article-of-manufacture of claim 42, wherein said electropolymerized polymer is selected from the group consisting of polypyrrole, polythiophene, poly-p-phenylene, poly-p-phenylene sulfide, polyaniline, poly(2,5-thienylene), fluoroaluminum, fluorogallium, phtalocyanine, derivatives thereof and any combination thereof.

49. The article-of-manufacture of claim 42, wherein said electropolymerized polymer comprises a bioactive agent being absorbed, swelled or embedded therein.

50. An article-of-manufacture comprising an object having a conductive surface and a self-assembled monolayer of at least one organic substance being attached to at least a portion of said conductive surface.

51. The article-of-manufacture of claim 50, wherein said organic substance comprises an electroattachable group and said self-assembled monolayer is electrochemically formed onto said conductive surface.

52. The article-of-manufacture of claim 50, wherein said organic substance is an organosilane and said self-assembled monolayer is non-electrochemically formed onto said conductive surface.

53. The article-of-manufacture of claim 52, wherein said organosilane has the general formula:
XmSiR(4-m)
whereas:
m is an integer from 1 to 3;
X is selected from the group consisting of halide, alkoxy and thioalkoxy; and
R is a substituted or unsubstituted, saturated or unsaturated hydrocarbon residue.

54. The article-of-manufacture of claim 53, wherein said hydrocarbon residue has from 1 to 10 carbon atoms.

55. The article-of-manufacture of claim 51, wherein said electroattachable group is selected from the group consisting of a carboxylate, a sulfonate, a sulfate, a phosphonate and a phosphate.

56. The article-of-manufacture of claim 51, wherein said organic substance further comprises an organic residue having 3-30 carbon atoms.

57. The article-of-manufacture of claim 56, wherein said organic substance is a fatty acid.

58. The article-of-manufacture of claim 57, wherein said fatty acid is selected from the group consisting of decanoic acid, myristic acid, palmitic acid, and stearic acid.

59. The article-of-manufacture of claim 50, wherein said organic substance further comprises at least one functional group capable of interacting with at least one active substance.

60. The article-of-manufacture of claim 57, wherein said fatty acid is derivatized by at least one functional group capable of interacting with at least one active substance.

61. The article-of-manufacture of claim 53, wherein at least one hydrocarbon residue is substituted by at least one functional moiety capable of interacting with at least one active substance.

62. The article-of-manufacture of claims 59, 60 and 61, wherein said at least one functional group is selected from the group consisting of amine, ammonium ion, carboxylate, thiocarboxylate, amide, carbamyl, hydroxyl, thiohydroxyl, alkoxide, thioalkoxide, nitrate, cyanate, pyrrole, isocyanate, halide, azide, an unsaturated moiety, a hydrophobic moiety, phosphate, phosphonate, sulfate, sulfonate, sulfonamide, and any combination thereof.

63. The article-of-manufacture of claims 59, 60 and 61, wherein said interacting is effected by a covalent bond, a biodegradable bond, a ionic bond, a hydrogen bond, Van der Waals interactions, hydrophobic interactions, swelling and absorption.

64. The article-of-manufacture of claims 59-63, further comprising at least one active substance being attached to said at least one functional group.

65. The article-of-manufacture of claim 50, further comprising an electropolymerized polymer being attached to at least a portion of said conductive surface.

66. The article-of-manufacture of claim 65, wherein said electropolymerized polymer comprises a bioactive agent attached thereto.

67. The article-of-manufacture of claim 65, wherein said electropolymerized polymer comprises a plurality of microparticles and/or nanoparticles attached thereto.

68. The article-of-manufacture of claim 67, wherein said plurality of microparticles and/or nanoparticles comprises a bioactive agent being attached thereto or encapsulated therein.

69. The article-of-manufacture of claim 65, wherein said electropolymerized polymer comprises a co-polymer attached thereto.

70. The article-of-manufacture of claim 69, wherein said co-polymer comprises a bioactive agent being attached thereto or encapsulated therein.

71. The article-of-manufacture of claim 65, wherein said electropolymerized polymer comprises a bioactive agent being absorbed, swelled or embedded therein.

72. The article-of-manufacture of claim 65, wherein said electropolymerized polymer is selected from the group consisting of polypyrrole, polythiophene, poly-p-phenylene, poly-p-phenylene sulfide, polyaniline, poly(2,5-thienylene), fluoroaluminum, fluorogallium, phtalocyanine, derivatives thereof and any combination thereof.

73. A process of preparing an object having a conductive surface and at least one active substance being attached to at least a portion of said conductive surface, the process comprising:
providing said object having said conductive surface;
modifying said conductive surface to thereby provide an object having a conductive surface having at least one functional moiety attached thereto, said at least one functional moiety being capable of interacting with said at least one active substance; and
contacting said active substance and said conductive surface having at least one functional moiety attached thereto.

74. The process of claim 73, wherein said interacting is effected by a covalent bond, a biodegradable bond, a ionic bond, a hydrogen bond, Van der Waals interactions, hydrophobic interactions, swelling and absorption.

75. The process of claim 74, wherein said modifying is effected by attaching to said conductive surface at least one organic substance, said organic substance comprising and a functional moiety capable of interacting with said active substance.

76. The process of claim 75, wherein said modifying is effected by electrochemically attaching to said conductive surface at least one organic substance, said organic substance comprising an electroattachable group and a functional moiety capable of interacting with said active substance.

77. The process of claim 75, wherein said organic substance is organosilane and said modifying is effected by non-electrochemically attaching to said conductive surface said organosilane.

78. The process of claim 77, wherein said organosilane has the general formula:
XmSiR(4-m)
whereas:
m is an integer from 1 to 3;
X is selected from the group consisting of halide, alkoxy and thioalkoxy; and
R is a substituted or unsubstituted, saturated or unsaturated hydrocarbon residue.

79. The process of claim 78, wherein said hydrocarbon residue has from 1 to 10 carbon atoms.

80. The process of claim 75, wherein said at least one organic substance forms a self-assembled monolayer onto said conductive surface.

81. The process of claim 73, wherein said contacting is effected by reacting said conductive surface having said at least one functional moiety attached thereto and said active substance.

82. The process of claim 73, wherein said contacting is effected by swelling said active substance within said conductive surface having at least one functional moiety attached thereto.

83. The process of claim 73, wherein said active substance is a polymer and said contacting is effected by polymerizing a monomer corresponding to said polymer onto said conductive surface having at least one functional moiety attached thereto.

84. The process of claim 83, wherein said polymer is an electropolymerizable polymer and said contacting is effected by polymerizing an electropolymerizable monomer corresponding to said polymer onto said conductive surface having at least one functional moiety attached thereto.

85. The process of claim 73, wherein said contacting is effected by absorbing said active substance to said conductive surface having at least one functional moiety attached thereto.

86. A process of preparing an object having a conductive surface and at least one active substance being attached to at least a portion of said conductive surface, the process comprising:
providing said object having said conductive surface; and
electrochemically attaching to said conductive surface at least one active substance having an electroattachable group.

87. The process of claim 86, wherein said object is a medical device.

88. The process of claim 86, wherein said object is an implantable medical device.

89. The process of claim 88, wherein said object is a stent.

90. The process of claim 86, wherein said at least one active substance is selected from the group consisting of a bioactive agent, a polymer, a polymer having a bioactive agent attached thereto, a plurality of microparticles and/or nanoparticles, a plurality of microparticles and/or nanoparticles having a bioactive agent attached thereto, and any combination thereof, said bioactive agent, polymer, nanoparticles and/or microparticles comprising said electroattachable group.

91. The process of claim 90, wherein said bioactive agent is selected from the group consisting of a therapeutically active agent and a labeling agent.

92. The process of claim 86, further comprising, prior to said electrochemically attaching, modifying said conductive surface, to thereby provide an object having a conductive surface having at least one functional moiety capable of interacting with said at least one active substance.

93. The process of claim 92, wherein said modifying comprises electrochemically modifying said conductive surface.

94. The process of claim 92, wherein said interacting is effected by a covalent bond, a biodegradable bond, a ionic bond, a hydrogen bond, Van der Waals interactions, hydrophobic interactions, swelling and absorption.

95. The process of claims 86 and 90, wherein said electroattachable group is selected from the group consisting of a carboxylate, a sulfonate, a sulfate, a phosphonate and a phosphate.

96. The process of claim 86, wherein said active substance is an electropolymerized polymer.

97. The process of claim 91, wherein said therapeutically active agent is selected from the group consisting of an anti-thrombogenic agent, an anti-platelet agent, an anti-coagulant, a growth factor, a statin, a toxin, an antimicrobial agent, an analgesic, an anti-metabolic agent, a vasoactive agent, a vasodilator agent, a prostaglandin, a hormone, a thrombin inhibitor, an enzyme, an oligonucleotide, a nucleic acid, an antisense, a protein, an antibody, an antigen, a vitamin, an immunoglobulin, a cytokine, a cardiovascular agent, endothelial cells, an anti-inflammatory agent, an antibiotic, a chemotherapeutic agent, an antioxidant, a phospholipid, an anti-proliferative agent, a corticosteroid, a heparin, a heparinoid, albumin, a gamma globulin, paclitaxel, hyaluronic acid and any combination thereof.

98. A method of treating a subject having a medical condition in which implanting a medical device is beneficial, the method comprising:
providing a medical device having a conductive surface and an active substance being attached at least to a portion of said conductive surface, wherein said conductive surface is a modified conductive surface having at least one functional moiety capable of interacting with said at least one active substance and/or said at least one active substance is electrochemically attached to said conductive surface; and
implanting said medical device within said subject, thereby treating said medical condition.

99. The method of claim 98, wherein said medical condition is selected from the group consisting of a cardiovascular disease, atherosclerosis, thrombosis, stenosis, restenosis, a cardiologic disease, a peripheral vascular disease, an orthopedic condition, a proliferative disease, an infectious disease, a transplantation-related disease, a degenerative disease, a cerebrovascular disease, a gastrointestinal disease, a hepatic disease, a neurological disease, an autoimmune disease, and an implant-related disease.

100. The method of claim 98, wherein said medical device is an implantable device.

101. The method of claim 98, wherein said medical device is selected from the group consisting of a pacemaker, a graft, a stent, a wire, an orthopedic implant, an implantable diffusion pump, an injection port and a heart valve.

102. The method of claim 101, wherein said medical device is a stent.

103. The method of claim 98, wherein said at least one active substance is selected from the group consisting of a bioactive agent, a polymer, a polymer having a bioactive agent attached thereto, a plurality of microparticles and/or nanoparticles, a plurality of microparticles and/or nanoparticles having a bioactive agent attached thereto, and any combination thereof.

104. The method of claim 103, wherein said bioactive agent is selected from the group consisting of a therapeutically active agent and a labeling agent.

105. The method of claim 104, wherein said therapeutically active agent is selected from the group consisting of an anti-thrombogenic agent, an anti-platelet agent, an anti-coagulant, a growth factor, a statin, a toxin, an antimicrobial agent, an analgesic, an anti-metabolic agent, a vasoactive agent, a vasodilator agent, a prostaglandin, a hormone, a thrombin inhibitor, an enzyme, an oligonucleotide, a nucleic acid, an antisense, a protein, an antibody, an antigen, a vitamin, an immunoglobulin, a cytokine, a cardiovascular agent, endothelial cells, an anti-inflammatory agent, an antibiotic, a chemotherapeutic agent, an antioxidant, a phospholipid, an anti-proliferative agent, a corticosteroid, a heparin, a heparinoid, albumin, a gamma globulin, paclitaxel, hyaluronic acid and any combination thereof.

106. Use of the implantable device of claim 3 in the treatment of a medical condition in which implanting a device having said active substance attached to a surface thereof is beneficial.

107. The use of claim 106, wherein said medical condition is selected from the group consisting of a cardiovascular disease, atherosclerosis, thrombosis, stenosis, restenosis, a cardiologic disease, a peripheral vascular disease, an orthopedic condition, a proliferative disease, an infectious disease, a transplantation-related disease, a degenerative disease, a cerebrovascular disease, a gastrointestinal disease, a hepatic disease, a neurological disease, an autoimmune disease, and an implant-related disease.

108. The use of claim 106, wherein said at least one active substance is selected from the group consisting of a bioactive agent, a polymer, a polymer having a bioactive agent attached thereto, a plurality of microparticles and/or nanoparticles, a plurality of microparticles and/or nanoparticles having a bioactive agent attached thereto, and any combination thereof, said bioactive agent, said polymer, said microparticles and/or said nanoparticles comprising at least one electroattachable group.

109. The use of claim 108, wherein said therapeutically active agent is selected from the group consisting of a therapeutically active agent and a labeling agent.

110. The use of claim 109, wherein said therapeutically active agent is selected from the group consisting of an anti-thrombogenic agent, an anti-platelet agent, an anti-coagulant, a growth factor, a statin, a toxin, an antimicrobial agent, an analgesic, an anti-metabolic agent, a vasoactive agent, a vasodilator agent, a prostaglandin, a hormone, a thrombin inhibitor, an enzyme, an oligonucleotide, a nucleic acid, an antisense, a protein, an antibody, an antigen, a vitamin, an immunoglobulin, a cytokine, a cardiovascular agent, endothelial cells, an anti-inflammatory agent, an antibiotic, a chemotherapeutic agent, an antioxidant, a phospholipid, an anti-proliferative agent, a corticosteroid, a heparin, a heparinoid, albumin, a gamma globulin, paclitaxel, hyaluronic acid and any combination thereof.

111. A system for coating at least one medical device having a conductive surface, the system comprising in operative arrangement, at least one holding device for holding said at least one medical device, a conveyer, and a first and second bath arranged along said conveyer, wherein said conveyer is designed and constructed to convey said at least one holding device such that said at least one holding device is placed within each of said first and second baths for a predetermined time period and in a predetermined order, and further wherein said first bath is a modification bath and said second bath is an active substance solution bath.

112. The system of claim 111, wherein said modification bath comprises an organic substance having a functional moiety capable of interacting with said active substance.

113. The system of claim 112, wherein said active substance is an electropolymerized polymer and said second bath is an electropolymerization bath.

114. The system of claim 111, wherein said at least one medical device comprises at least one stent assembly.

115. The system of claim 111, further comprising at least one additional bath arranged along said conveyer, wherein said conveyer is designed and constructed to place said at least one holding device within said at least one additional treating bath for a predetermined time period.

116. The system of claim 115, wherein said at least one additional treating bath is selected from the group consisting of a pretreatment bath, a washing bath, a rinsing bath, an electropolymerization bath, a chemical polymerization bath and a second active substance solution bath.

117. The system of claim 111, further comprising a cartridge having a cartridge body adapted for enabling said at least one holding device to be mounted onto said cartridge body.

118. The system of claim 116, wherein said at least one holding device comprises a perforated encapsulation, adapted to receive said at least one medical device, and at least two cups adapted for enabling electrode structures to engage with said perforated encapsulation hence to generate an electric field within said perforated encapsulation.

119. The system of claim 118, wherein said perforated encapsulation is designed and constructed to allow fluids and chemicals to flow therethrough.

120. The system of any of claims 113 and 118, wherein said electropolymerization bath comprises at least one electrode structure, mounted on a base of said electropolymerization bath and connected to an external power source.

121. The system of claim 120, wherein said conveyer is operable to mount said at least one holding device on said at least one electrode structure, thereby to engage said at least one electrode structure with a first side of said perforated encapsulation.

122. The system of claim 119, further comprising an arm carrying at least one electrode structure and operable to engage said at least one electrode structure with a second side of said perforated encapsulation.
